(19) United States
(12) Patent Application Publication
(10) Pub. No.: US 2010/0063047 A1
(43) Pub. Date: Mar. 11, 2010

(54) AMINOPYRIMIDINE INHIBITORS OF HISTAMINE RECEPTORS FOR THE TREATMENT OF DISEASE

(75) Inventors: Allen J. Borchardt, San Diego, CA (US); Clay Beauregard, Fort Worth, TX (US); Robert L. Davis, Carlsbad, CA (US); Daniel A. Gamache, Arlington, TX (US); John M. Yanni, Burleson, TX (US)

Correspondence Address: GLOBAL PATENT GROUP - KAL, 10411 Clayton Road, Suite 304, St. Louis, MO, 63131, US

(73) Assignees: KALYPSYS, INC., San Diego, CA (US); ALCON RESEARCH, LTD, Fort Worth, TX (US)

(21) Appl. No.: 12/556,842

(22) Filed: Sep. 10, 2009

Related U.S. Application Data
(60) Provisional application No. 61/095,819, filed on Sep. 10, 2008.

(51) Int. Cl. A61K 31/5377; C07D 495/04; A61K 31/497; A61K 31/519; A61K 31/4365

(52) U.S. Cl. 514/234.2; 544/278; 514/252.16; 514/260.1; 544/117; 546/114; 514/301

(57) ABSTRACT

The present invention relates to compounds and methods which may be useful as inhibitors of H₁R and/or H₄R for the treatment or prevention of inflammatory, autoimmune, allergic, and ocular diseases.

This application claims the benefit of priority of U.S. provisional application No. 61/095,819, filed Sep. 10, 2008, the disclosure of which is hereby incorporated by reference as if written herein in its entirety.

Disclosed herein are new heterocyclic compounds and compositions and their application as pharmaceuticals for the treatment of disease. Methods of inhibition of histamine receptor activity in a human or animal subject are also provided for the treatment of allergic diseases, inflammation, asthma, rhinitis, chronic obstructive pulmonary disease, conjunctivitis, rheumatoid arthritis, and general and localized pruritis.

Histamine, a low molecular weight biogenic amine, is a potent chemical mediator of normal and pathological physiology. Histamine functions as a secreted signal in immune and inflammatory responses, as well as a neurotransmitter. The functions of histamine are mediated through 4 distinct cell surface receptors (H₁R, H₂R, H₃R and H₄R). Histamine receptors vary in expression, signaling, function and histamine affinity, and therefore have different potential therapeutic applications (Zhang M, Thurmond R L, and Dunford P J Pharmacology & Therapeutics. 2007).

All 4 histamine receptors are G protein-coupled receptors (GPCRs). Upon histamine or other agonist binding, they activate distinct signaling pathways through different heterotrimeric G proteins. The H₁R couples to the G_{q} family of G proteins, whose primary signaling cascade induces second messenger calcium mobilization from intracellular stores, followed by multiple downstream effects. H₁R can also increase cyclic GMP (cGMP) production and activate NFκB, a potent, positive transcriptional regulator of inflammation. The H₂R couples to the Gₛ family of G proteins and increases cyclic AMP (cAMP) formation by stimulating adenylate cyclase, although it can also induce calcium mobilization in some cell types. The H₃R mediates its function through G_{i/o} proteins and decreases cAMP formation by inhibiting adenylate cyclase. Like other G_{i/o}-coupled receptors, H₃R also activates the mitogen-activated protein/extracellular-signal regulated protein (MAP/ERK) kinase pathway. H₄Rhas also been demonstrated to couple to G_{i/o} proteins, with canonical inhibition of cAMP formation and MAP kinase activation. However, H₄R also couples to calcium mobilization in certain cell types. In fact, H₄R signaling in mast cells is primarily through calcium mobilization with little to no impact on cAMP formation.

The H₁R is expressed in many cell types, including endothelial cells, most smooth muscle cells, cardiac muscle, central nervous system (CNS) neurons, and lymphocytes. H₁R signaling causes smooth muscle contraction (including bronchoconstriction), vasodilation, and increased vascular permeability, hallmarks of allergic and other immediate hypersensitivity reactions. In the CNS, H₁R activation is associated with wakefulness. Its activation is also associated with pruritus and nociception in skin and mucosal tissues. For many years, the anti-allergic and anti-inflammatory activities of H₁R antagonists have been utilized to treat acute and chronic allergic disorders and other histamine-mediated pathologies, such as itch and hives.

The H₂R is expressed similarly to the H₁R, and can also be found in gastric parietal cells and neutrophils. H₂R is best known for its central role in gastric acid secretion but has also been reported to be involved in increased vascular permeability and airway mucus production. Antagonists of H2R are widely used in treating peptic ulcers and gastroesophageal reflux disease. These drugs are also used extensively to reduce the risk of gastrointestinal (GI) bleeding associated with severe upper GI ulcers and GI stress in the inpatient setting.

The H₃R is primarily found in the CNS and peripheral nerves innervating cardiac, bronchial, and GI tissue. H₃R signaling regulates the release of multiple neurotransmitters, such as acetylcholine, dopamine, serotonin, and histamine itself (where it acts as a CNS autoreceptor). In the CNS, H₃R participates in the processes of cognition, memory, sleep, and feeding behaviors. H₃R antagonists may be used potentially for treating cognition disorders (such as Alzheimer's disease), sleep and wakefulness disorders, attention disorders, and metabolic disorders (especially related to obesity).

Existence of the H₄R was predicted in the early 1990s, but its cloning by multiple groups was not reported until 2000. In contrast to the other histamine receptors, the H₄R has a distinctly selective expression profile in bone marrow and on certain types of hematopoietic cells. H₄R signaling modulates the function of mast cells, eosinophils, dendritic cells, and subsets of T cells. The H₄R appears to control multiple behaviors of these cells, such as activation, migration, and cytokine and chemokine production (Zhang M, Thurmond R L, and Dunford P J Pharmacology & Therapeutics. 2007).

Of the 4 known histamine receptors, H₁R, H₂R and H₄R have been shown clearly to affect inflammation and other immune responses and are proposed therapeutic targets for treating immune and inflammatory disorders (Jutel et al., 2002; Akdis & Simons, 2006). The H₁R was the first described histamine receptor, and ligands targeting this receptor were initially developed in the 1930s and in widespread use by the 1940s. Common H₁R antagonist drugs currently approved for use include systemic agents such as diphenhydramine (Benadryl, also used topically), cetirizine (Zyrtec), fexofenadine (Allegra), loratadine (Claritin) and desloratadine (Clarinex), and topical agents such as olopatadine (Patanol, Pataday, Patanase), ketotifen, azelastine (Optivar, Astelin) and epinastine (Elestat). Traditional uses have included allergic diseases and reactions such as asthma, rhinitis, and other chronic obstructive pulmonary disorders, ocular disorders such as allergic conjunctivitis, and pruritis of varying etiologies.

However, H₁ receptor antagonists have certain deficiencies as therapeutic agents in the treatment of diseases where histamine is an important mediator. First, their effects are often only moderate and reduce allergic symptoms by only 40 to 50%. In particular, H₁ receptor antagonists, especially systemic agents, have little to no effect in relieving nasal congestion. In allergic asthma, despite the fact that histamine levels rapidly increase in the airways and in plasma (correlating with disease severity), H₁ receptor antagonists have largely failed as a therapeutic strategy, though some effect is seen with administration during the priming phase as opposed to the challenge phase (Thurmond R L et al., Nat Rev Drug Discov, 2008, 7:41-53). Additionally, although the efficacy of H₁ receptor antagonists against pruritus in acute urticarias, associated with hives and insect stings, and in chronic idiopathic urticaria is well proven, H₁R antagonists are mostly ineffective in the treatment of atopic dermatitis-associated pruritus, with the only modest benefits derived from some first-generation compounds likely a consequence of their sedative properties (Sharpe, G. R. & Shuster, S. Br. I Dermatol. 1993, 129:575-9). Finally, sedation caused by H₁R antagonists that cross the blood-brain barrier, among other side effects, limits the utility of many H₁R antagonists in diseases for which they would otherwise be efficacious. These deficiencies render H₁R antagonists amenable to replacement by or supplementation with other agents.

Consequently, attention has focused on the more recently discovered H₄ receptor as a therapeutic target. Given the ability of H₄R to modulate the cellular function of eosinophils, mast cells, dendritic cells and T cells (M. Zhang et al., Pharmacol Ther 2007), it is natural to speculate that the H₄R may be involved in various inflammatory diseases, and that H₄R antagonists would have therapeutic potential (Jutel et al., 2006). Indeed, both in vitro and in vivo evidence has been demonstrated for the utility of H₄R antagonists as anti-inflammatory agents in inflammatory bowel disease (IBD) (Sander L E et al., Gut 2006; 55:498-504). The finding that H₄ receptor antagonists inhibit histamine-induced migration of mast cells and eosinophils in vitro and in vivo, both of which are important effector cells in the allergic response, raises the possibility that this class of compounds could reduce the allergic hyper-responsiveness developed upon repeated exposure to antigens, which is characterized by an increase in the number of mast cells and other inflammatory cells in the nasal and bronchial mucosa (Fung-Leung W P et al., Curr Opin Inves Drugs, 2004 5:11 1174-1182). In contrast to some of the H₁R antagonists, H₄R antagonists given during the allergen challenge phase of a mouse model of asthma are equally effective to those given during sensitization (Thurmond R L et al., Nat Rev Drug Discov, 2008, 7:41-53). In two recent mouse studies, a selective H₄R agonist was shown to induce itch, whereas these responses, and those of histamine, were blocked by pretreatment with H₄R antagonists. Similarly, histamine or H₄ receptor agonist-induced itch was markedly attenuated in H₄ receptor-deficient animals (Dunford, P. J. et al., J. Allergy Clin. Immunol, 2007, 119:176-183). The presence of the H₄R in nasal tissue was first discovered by Nakaya et al. (Nakaya, M. et al., Ann Otol Rhinol Laryngol, 2004, 113: 552-557). In addition, a more recent finding showed that there is a significant increase in the level of H₄R in human nasal polyp tissue taken from patients with chronic rhinosinusitis (infection of the nose and nasal cavities) when compared to normal nasal mucosa. Jóküti et al. suggest that the administration of H₄R antagonists might be a new way to treat nasal polyps and chronic rhinosinusitis. The administration of H₄R antagonists may prevent the accumulation of eosinophils as a result of impaired cell chemotaxis toward polypous tissue (Jóküti, A. et al., Cell Biol Int, 2007, 31: 1367). Although scientific data on the role of the H₄R in rhinitis is limited, at present, it is the only indication for which an H₄R inverse agonist (CZC-13788) is reported to be in preclinical development (Hale, R. A. et al., Drug News Perspect, 2007, 20: 593-600).

Current research efforts include both a focus on H₄R selective agents and an alternate path toward dual H₁R/H₄R agents. Johnson & Johnson have developed a well-characterized H₄R antagonist, JNJ-7777120, which is 1000-fold selective over H₁, H₂, and H₃ receptors, and equipotent across human and several nonhuman species. An exemplary H₁R/H₄R dual agent has yet to publish as of the time of this writing, and the ideal proportion of H₁R versus H₄R antagonism is a nascent topic of debate. Nevertheless, the concept of dual activity via a single agent is well-precedented, and the design of multiply active ligands is a current topic in pharmaceutical discovery (Morphy R and Rankovic Z, J Med Chem. 2005; 48(21):6523-43). Additional reports have shown potential for H₄R antagonists, or potentially, H₁R/H₄R dual antagonists, in the treatment of metabolic disorders such as obesity (Jorgensen E et al., Neuroendocrinology. 2007; 86(3):210-4), vascular or cardiovascular diseases such as atherosclerosis (Tanihide A et al., TCM 2006: 16(8): 280-4), inflammation and pain (Coruzzi G et al., Eur J Pharmacol. 2007 Jun. 1;563(1-3):240-4), rheumatoid arthritis (Grzybowska-Kowalczyk A et al., Inflamm Res. 2007 April;56 Suppl 1:S59-60) and other inflammatory and autoimmune diseases including systemic lupus erythematosus (Zhang M, Thurmond R L, and Dunford P J Pharmacology & Therapeutics. 2007). What is clear is that a need still exists in the art for improved and varied antihistamines for the treatment of disease, and that compounds with H₄R and/or H₁R/H₄R antagonist activity may fill this need.

Histamine is reportedly implicated in allergic rhinitis by acting on three HR subtypes, the H₁R, H₃R and H₄R. For many years, the classical application of H₁R antagonists (antihistamines) has been the treatment of allergic rhinitis. H₁R antagonists relieve edema and vasoconstriction, both important symptoms of the disease, but these drugs do not affect the underlying inflammatory responses. After the discovery of the H₃R and H₄R subtypes, the traditional role for H₁R antagonists in rhinitis has been reappraised. It has been shown that the H₃R agonist (R)-α-methyl-histamine (2) can induce the dilatation of nasal blood vessels and that this effect can be counteracted by the H₃R antagonist/H₄R agonist clobenpropit (Taylor-Clark, T., et al, Pulm Pharm Ther, 2008, 21: 455-460). Although a role for the H₄R cannot be ruled out, this H₃R antagonist-mediated mechanism in nasal decongestion has certainly caught the attention of scientists from Pfizer Inc. Recently, patient recruitment started for a Phase II clinical trial to test a H₃R antagonist (PF-03654746, unpublished structure) as a novel nasal decongestant in patients with seasonal allergic rhinitis. A dual target approach is being pursued by GSK that is currently recruiting patients to test a systemic H₁/H₃ antagonist (GSK835726, unpublished structure) for seasonal allergic rhinitis in a Phase I clinical trial. A second Phase I trial with another H₁/H₃ antagonist (GSK1004723, unpublished structure) for intranasal administration to treat rhinitis has recently been completed. With these compounds, the mode of action of the classical H₁R antagonist is combined with the potential clinical benefit of added nasal decongestion by H₃R blockade. The synergistic role of the H₁R and H₃R has been demonstrated in vivo in experiments performed at Schering-Plough. In view of the role of the H₄R in allergic rhinitis, other potential treatment paradigms may also be considered, such as combining H₁/H₄, H₃/H₄ or even H₁/H₃/H₄ antagonists/inverse agonist activity in the same molecule approach is being pursued by GSK that is currently recruiting patients to test a systemic H₁/H₃ antagonist (GSK835726, unpublished structure) for seasonal allergic rhinitis in a Phase I clinical trial. A second Phase I trial with another H₁/H₃ antagonist (GSK1004723, unpublished structure) for intranasal administration to treat rhinitis has recently been completed. With these compounds, the mode of action of the classical H₁R antagonist is combined with the potential clinical benefit of added nasal decongestion by H₃R blockade. The synergistic role of the H₁R and H₃R has been demonstrated in vivo in experiments performed at Schering-Plough (McLeod, R. et al., Am J Rhinol, 1999, 3: 391-399). In view of the role of the H₄R in allergic rhinitis, other potential treatment paradigms may also be considered, such as combining H₁/H₄, H₃/H₄ or even H₁/H₃/H₄ antagonists/inverse agonist activity in the same molecule.

Novel compounds and pharmaceutical compositions, certain of which have been found to inhibit the histamine type-1 receptor (H₁R) and/or the histamine type-4 receptor (H₄R) have been discovered, together with methods of synthesizing and using the compounds including methods for the treatment of histamine receptor-mediated diseases in a patient by administering the compounds.

In certain embodiments of the present invention, compounds have structural Formula I:

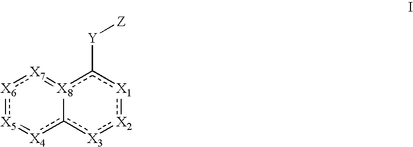

or a salt thereof, wherein:

a dashed line indicates that a bond may be present or absent;

X₁ and X₃ are independently selected from the group consisting of [C(R₂)(R₃)] and NR₄;

X₂ is selected from the group consisting of [C(R₅)(R₆)], NR₇, O, and S;

X₄ is selected from the group consisting of [C(R₈)(R₉)], NR₁₀, O, and S;

X₅ is selected from the group consisting of [C(R₁₁)(R₁₂)], NR₁₃, O, and S;

X₆ is selected from the group consisting of [C(R₁₄)(R₁₅)], NR₁₆, O, and S;

X₇ is selected from the group consisting of [C(R₁₇)(R₁₈)], NR₁₉, O, S, and a bond;

X₈ is selected from the group consisting of C and N;

taken together, X₁ to X₈ form a fully aromatic bicyclic system;

Y is selected from the group consisting of a bond, NR₁[C(R₂₀)(R₂₁)]ₙ, NR₁[C(R₂₂)(R₂₃)]ₙ—W—[C(R₂₄)(R₂₅)]ₘ, S—[C(R₂₆)(R₂₇)]ₙ—W—[C(R₂₈)(R₂₉)]ₘ, O[C(R₃₀)(R₃₁)]ₙ, [C(R₃₂)(R₃₃)]ₙ—W—[C(R₃₄)(R₃₅)]ₘ, and [C(R₃₆)(R₃₇)]ₙ;

n and m are each independently an integer from 0 to 3;

W is selected from the group consisting of O, S, S(O)₂, NR₃₈, NR₃₉S(O₂), C(O), C(S), C(O)O, C(O)NR₄₀, NR₄₁C(O), and NR₄₂C(O)O;

Z is selected from the group consisting of hydrogen, aryl, alkyl, heterocycloalkyl, and cycloalkyl, any of which may be optionally substituted;
R₁ to R₄₂ are each independently selected from the group consisting of null, hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
R₁₁ and R₁₄ may be joined together to form a partially saturated cycloalkyl; and
R₁ and R₂₀, or R₁ and R₂₂, or R₂₂ and R₃₈, or R₁ and R₃₈, may be joined together to form a heterocycloalkyl.

Certain compounds disclosed herein may possess useful histamine receptor inhibitory activity, and may be used in the treatment or prophylaxis of a disease or condition in which H₁R and/or H₄R plays an active role. Thus, in broad aspect, certain embodiments also provide pharmaceutical compositions comprising one or more compounds disclosed herein together with a pharmaceutically acceptable carrier, as well as methods of making and using the compounds and compositions. Certain embodiments provide methods for inhibiting H₁R and/or H₄R. Other embodiments provide methods for treating a H₁R- and/or H₄R-mediated disorder in a patient in need of such treatment, comprising administering to said patient a therapeutically effective amount of a compound or composition according to the present invention. Also provided is the use of certain compounds disclosed herein for use in the manufacture of a medicament for the treatment of a disease or condition ameliorated by the inhibition of H₁R and/or H₄R.

In certain embodiments of the present invention, compounds have structural Formula II:

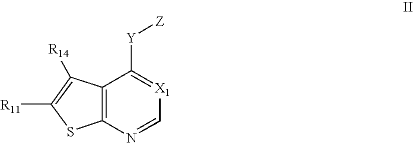

or a salt thereof, wherein:

X₁ is selected from the group consisting of [C(R₂)] and N;

Y is selected from the group consisting of a bond, NR₁[C(R₂₀)(R₂₁)]ₙ, NR₁[C(R₂₂)(R₂₃)]ₙ—W—[C(R₂₄)(R₂₅)]ₘ, S—[C(R₂₆)(R₂₇)]ₙ—W—[C(R₂₈)(R₂₉)]ₘ, O[C(R₃₀)(R₃₁)]ₙ, [C(R₃₂)(R₃₃)]ₙ—W—[C(R₃₄)(R₃₅)]ₘ, and [C(R₃₆)(R₃₇)]ₙ;

n and m are each independently an integer from 0 to 3;

W is selected from the group consisting of O, S, S(O)₂, NR₃₈, NR₃₉S(O₂), C(O), C(S), C(O)O, C(O)NR₄₀, NR₄₁C(O), and NR₄₂C(O)O;

Z is selected from the group consisting of aryl, alkyl, heterocycloalkyl, alkoxylcarbonyl, acyl, and cycloalkyl, any of which may be optionally substituted;
R₁, R₂, R₁₄, and R₂₀ to R₄₂ are each independently selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
R₁₁ is selected from the group consisting of hydrogen, alkyl, heteroalkyl, alkoxy, halogen, haloalkyl, amino, aminoalkyl, amido, carboxyl, acyl, hydroxy, cyano, nitro, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
R₁₁ and R₁₄ may be joined together to form a partially saturated cycloalkyl; and
R₁ and R₂₀, or R₁ and R₂₂, or R₂₂ and R₃₈, or R₁ and R₃₈, may be joined together to form a heterocycloalkyl;
and with the provisos that;
if Y is NR₁[C(R₂₀)(R₂₁)]ₙ, R₁ is hydrogen, and n is 0, then Z is not aryl or heteroaryl; and
if Y is NR₁[C(R₂₂)(R₂₃)]ₙ—W—[C(R₂₄)(R₂₅)]ₘ, n is 2, m is 0, W is NR₃₈, R₂₂, and R₂₃ are hydrogen, and R₁ and R₃₈ are joined together to form a piperazine ring, then Z is not phenyl or methyl.

In further embodiments, X₁ is N; Y is selected from the group consisting of a bond, NR₁[C(R₂₀)(R₂₁)]ₙ, and NR₁[C(R₂₂)(R₂₃)]ₙ—W—[C(R₂₄)(R₂₅)]ₘ; and W is NR₃₈.

In yet further embodiments, R₁₁ and R₁₄ are each independently selected from the group consisting of hydrogen and C₁-C₃ alkyl.

In yet further embodiments, R₁₁ is hydrogen; and R₁₄ is methyl
In further embodiments, Y is NR₁[C(R₂₀)(R₂₁)]ₙ; n is an integer from 2 to 3; Z is

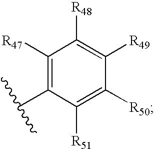

R₁, R₂₀, and R₂₁ are each independently selected from the group consisting of hydrogen and optionally substituted lower alkyl; and
R₄₇ to R₅₁ are each independently selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted; and any two adjacent R₄₇, R₄₈, R₄₉, R₅₀, or R₅₁ may join together to form a 5-, 6-, or 7-membered cycloalkyl or heterocycloalkyl.

In yet further embodiments, X₁ is N; n is 2; and R₁, R₂₀, and R₂₁ are each independently selected from the group consisting of hydrogen and methyl.

In yet further embodiments, R₁₁ and R₁₄ are each independently selected from the group consisting of hydrogen and C₁-C₃ alkyl; and R₄₇ to R₅₁ are each independently selected from the group consisting of hydrogen, alkyl, heteroalkyl, alkoxy, halogen, haloalkyl, amino, aminoalkyl, amido, carboxyl, acyl, hydroxy, cyano, nitro, and mercaptyl.

In yet further embodiments, R₁, R₁₁, R₂₀, and R₂₁ are each hydrogen; and R₁₄ is methyl.

In yet further embodiments, R₄₇ to R₅₁ are each independently selected from the group consisting of hydrogen, halogen, lower alkyl, and lower alkoxy.

In yet further embodiments, R₄₇, R₄₈, R₅₀, and R₅₁ are hydrogen; and R₄₉ is selected from the group consisting of hydrogen, halogen, methyl, and methoxy.

In yet further embodiments, R₄₉ is chlorine.

In certain embodiments of the present invention, compounds have a structural formula selected from the group consisting of structural Formula III and structural formula IV:

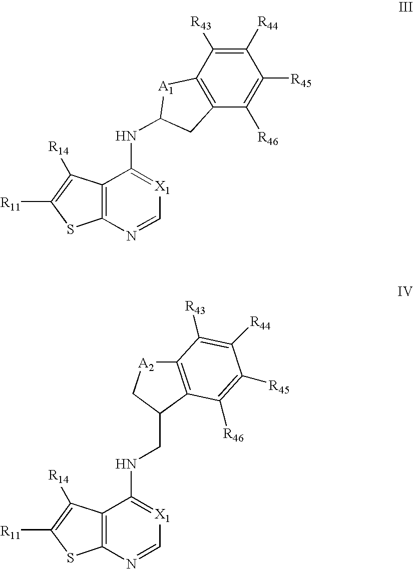

or a salt thereof, wherein:
A₁ and A₂ are each independently selected from the group consisting of a bond, —CH₂—, —CH₂CH₂—, and —CH₂CH₂CH₂—;
X₁ is selected from the group consisting of [C(R₂)] and N;
R₂, R₁₄, and R₄₃ to R₄₆ are each independently selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted; and

R₁₁ is selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted.

In further embodiments, A₁ and A₂ are each independently selected from the group consisting of —CH₂— and —CH₂CH₂—; X₁ is N; R₁₁ and R₁₄ are independently selected from the group consisting of hydrogen and C₁-C₃ alkyl; and R₄₃ to R₄₆ are each independently selected from the group consisting of hydrogen, alkyl, heteroalkyl, alkoxy, halogen, haloalkyl, amino, aminoalkyl, amido, carboxyl, acyl, hydroxy, cyano, nitro, and mercaptyl.

In yet further embodiments, A₁ and A₂ are —CH₂—; R₁₁ is hydrogen; R₁₄, is methyl; R₄₃ and R₄₆ are hydrogen; and R₄₄ and R₄₅ are each independently selected from the group consisting of hydrogen, lower alkyl, lower alkoxy, halogen, and lower haloalkyl.

In yet further embodiments, said compound has structural formula III; R₄₄ is hydrogen; and R₄₅ is halogen.

In yet further embodiments, R₄₅ is chlorine.

In yet further embodiments, said compound has structural formula IV; one of R₄₄ and R₄₅ is hydrogen; and the other of R₄₄ and R₄₅ is halogen.

In yet further embodiments, R₄₅ is chlorine.

In certain embodiments of the present invention, compounds have structural Formula V:

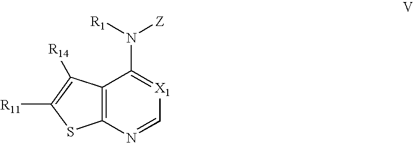

or a salt thereof, wherein:
X₁ is selected from the group consisting of [C(R₂)] and N;
Z is a 5- to 7-membered saturated cycloalkyl, which may be optionally substituted with one or more substituents selected from the group consisting of lower alkyl, lower alkanoyl, lower heteroalkyl, lower haloalkyl, lower perhaloalkyl, lower perhaloalkoxy, lower alkoxy, lower haloalkoxy, lower alkoxyalkyl, oxo, lower acyloxy, carboxyl, lower carboxyester, lower carboxamido, cyano, hydrogen, halogen, hydroxy, amino, lower alkylamino, amido, thiol, lower alkylthio, lower haloalkylthio, and lower perhaloalkylthio;

R₁, R₂, and R₁₄ are each independently selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted; and
R₁₁ is selected from the group consisting of hydrogen, alkyl, heteroalkyl, alkoxy, halogen, haloalkyl, amino, aminoalkyl, amido, carboxyl, acyl, hydroxy, cyano, nitro, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted.

In yet further embodiments, X₁ is N; R₁ is hydrogen; and R₁₁ and R₁₄ are independently selected from the group consisting of hydrogen and C₁-C₃ alkyl.

In yet further embodiments, Z is cyclohexyl, which may be optionally substituted with one or more substituents selected from the group consisting of lower alkyl, lower alkanoyl, lower heteroalkyl, lower alkoxy, oxo, lower acyloxy, carboxyl, lower carboxyester, and lower alkylamino.

In yet further embodiments, Z is cyclohexyl which may be optionally substituted in the 4-position with a substituent selected from the group consisting of lower alkyl and lower alkoxy; R₁₁ is hydrogen; and R₁₄ is methyl.

In yet further embodiments, Z is 4-alkylcyclohexyl.

In yet further embodiments, Z is 4-methylcyclohexyl.

In certain embodiments of the present invention, compounds have structural Formula VI:

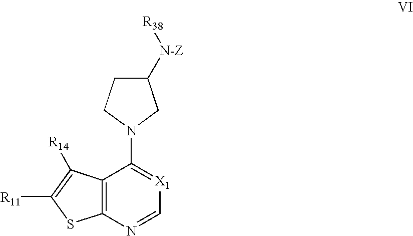

or a salt thereof, wherein:
X₁ is selected from the group consisting of [C(R₂)] and N;
Z is selected from the group consisting of hydrogen, aryl, alkyl, heterocycloalkyl, alkoxylcarbonyl, acyl, and cycloalkyl, any of which may be optionally substituted;
R₂, R₁₄, and R₃₄ are each independently selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;

R₁₁ is selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted; and

R₁₁ and R₁₄ may be joined together to form a partially saturated cycloalkyl.

In further embodiments, X₁ is N; and R₁₁ and R₁₄ are each independently selected from the group consisting of hydrogen and C₁-C₃ alkyl.

In yet further embodiments, R₁₁ is hydrogen; and R₁₄ is methyl.

In yet further embodiments, Z is selected from the group consisting of alkoxylcarbonyl and acyl; and R₃₄ is lower alkyl.

In certain embodiments of the present invention, compounds have a structural formula selected from the group consisting of structural Formula III and structural formula IV:

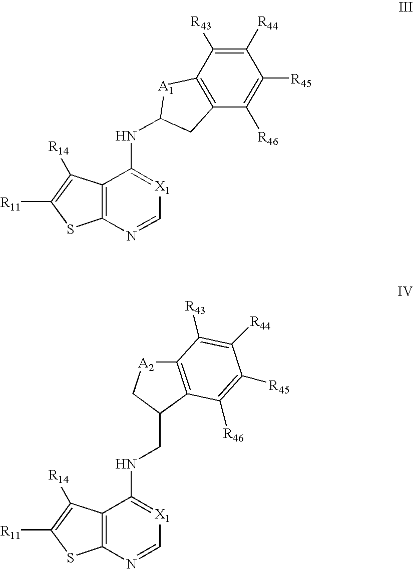

or a salt thereof, wherein:
A₁ and A₂ are each independently selected from the group consisting of a bond, —CH₂—, —CH₂CH₂—, and —CH₂CH₂CH₂—;
X₁ is selected from the group consisting of [C(R₂)] and N;
R₂ is selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
R₁₁ is selected from the group consisting of hydrogen, alkyl, heteroalkyl, alkoxy, haloalkyl, amino, aminoalkyl, amido, carboxyl, acyl, hydroxy, cyano, nitro, arylalkyl, cycloalkyl, cycloalkylalkyl, heterocycloalkylalkyl, heteroaryl, heteroarylalkyl, mercaptyl, alkylsulfonyl, sulfonamide, and alkylsulfonamido, any of which may be optionally substituted;
R₁₄ is is selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
R₄₃ and R₄₆ are each independently selected from the group consisting of hydrogen, alkyl, heteroalkyl, C₂-C₆ alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;

R₄₄ and R₄₅ are each independently selected from the group consisting of hydrogen, alkyl, heteroalkyl, C₂-C₆ alkoxy, halogen, haloalkyl, amino, aminoalkyl, acyl, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted; and
with the proviso that;
if the compound has structural formula III, A₁ is —CH₂—, R₁₁ is hydrogen or methyl, and R₁₄ is hydrogen, methyl, or isopropyl, then at least one of R₄₃ to R₄₆ is not hydrogen.

In further embodiments, A₁ and A₂ are each independently selected from the group consisting of —CH₂— and —CH₂CH₂—; X₁ is N; R₁₁ and R₁₄ are each independently selected from the group consisting of hydrogen and C₁-C₃ alkyl; and R₄₃ to R₄₆ are each independently selected from the group consisting of hydrogen, alkyl, heteroalkyl, C₂-C₆ alkoxy, halogen, haloalkyl, amino, aminoalkyl, amido, carboxyl, acyl, hydroxy, cyano, nitro, and mercaptyl.

In yet further embodiments, A₁ and A₂ are —CH₂—; R₁₁ is hydrogen; R₁₄, is methyl; R₄₃ and R₄₆ are hydrogen; and R₄₄ and R₄₅ are each independently selected from the group consisting of hydrogen, lower alkyl, lower alkoxy, halogen, and lower haloalkyl.

In yet further embodiments, said compound has structural formula III; R₄₄ is hydrogen; and R₄₅ is halogen.

In yet further embodiments, R₄₅ is chlorine.

In yet further embodiments, said compound has structural formula IV; one of R₄₄ and R₄₅ is hydrogen; and the other of R₄₄ and R₄₅ is halogen.

In yet further embodiments, R₄₅ is chlorine.

In certain embodiments of the present invention, compounds have structural Formula II:

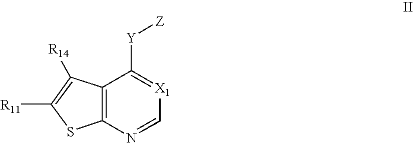

or a salt thereof, wherein:
X₁ is selected from the group consisting of [C(R₂)] and N;
Y is NR₁[C(R₂₀)(R₂₁)]ₙ;
n is an integer from 2 to 3;
Z is

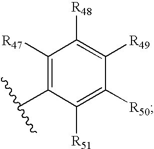

R_{1,} R₂₀, and R₂₁ are each independently selected from the group consisting of hydrogen and lower alkyl;
R₁₁ and R₁₄ are independently selected from the group consisting of hydrogen and C₁-C₃ alkyl;
R₂, R₄₇ to R₅₁ are each independently selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
any two adjacent R₄₇, R₄₈, R₄₉, R₅₀, or R₅₁, may be joined together to form a 5-, 6-, or 7-membered cycloalkyl or heterocycloalkyl;
with the provisos that;
if X₁ is [C(R₂)], R₁, R₂, R₂₀, and R₂₁ are hydrogen, R₁₁ is ethyl and R₁₄ is hydrogen, then at least one of R₄₇ to R₅₁ is not hydrogen;
if X₁ is N, then at least one of R₂₀ and R₂₁ is lower alkyl; and
if X₁ is N, R₁₁, R₁₄, and R₄₇ to R₅₁ are hydrogen, then Y is not —CH₂C(CH₃)₂—.

In further embodiments, X₁ is N; n is 2; and R₁, R₂₀, and R₂₁ are each independently selected from the group consisting of hydrogen and methyl.

In yet further embodiments, R₄₇ to R₅₁ are each independently selected from the group consisting of hydrogen, alkyl, heteroalkyl, alkoxy, halogen, haloalkyl, amino, aminoalkyl, amido, carboxyl, acyl, hydroxy, cyano, nitro, and mercaptyl.

In yet further embodiments, R₁ and R₁₁ are each hydrogen; and R₁₄ is methyl.

In yet further embodiments, R₄₇ to R₅₁ are each independently selected from the group consisting of hydrogen, halogen, lower alkyl, and lower alkoxy.

In yet further embodiments, R₄₇, R₄₈, R₅₀, and R₅₁ are hydrogen; and R₄₉ is selected from the group consisting of hydrogen, halogen, methyl, and methoxy.

In yet further embodiments, R₄₉ is chlorine.

In certain embodiments of the present invention, compounds have structural Formula V:

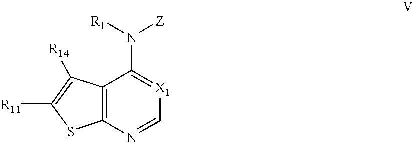

or a salt thereof, wherein:
X₁ is selected from the group consisting of [C(R₂)] and N;
Z is a 5- to 7-membered saturated cycloalkyl, which is substituted with at least one substituent selected from the group consisting of lower alkyl, lower alkanoyl, lower heteroalkyl, lower haloalkyl, lower perhaloalkyl, lower perhaloalkoxy, lower alkoxy, lower haloalkoxy, lower alkoxyalkyl, oxo, lower acyloxy, lower carboxyester, lower carboxamido, cyano, hydrogen, halogen, hydroxy, thiol, lower alkylthio, lower haloalkylthio, and lower perhaloalkylthio;
R₁ and R₂ are each independently selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, and alkylsulfonamido, any of which may be optionally substituted;
R₁₁ and R₁₄ are independently selected from the group consisting of hydrogen and C₁-C₃ alkyl;
with the provisos that;
if R₁₁ is methyl and R₁₄ is hydrogen, then Z is not 2,3-dimethylcyclohexyl;
if R₁₁ and R₁₄ are both hydrogen, if R₁₁ and R₁₄ are both methyl, or if R₁₁ is ethyl and R₁₄ is hydrogen, then Z is not 4-hydroxycyclohexyl;
if R₁₁ and R₁₄ are both hydrogen or if R₁₁ and R₁₄ are both methyl, then Z is not 2-methylcyclohexyl;
if R₁₁ and R₁₄ are both hydrogen or if R₁₁ and R₁₄ are both methyl, then Z is not 3-methylcyclohexyl; and
if R₁₁ and R₁₄ are both hydrogen or if R₁₁ and R₁₄ are both methyl, then Z is not 4-methylcyclohexyl.

In yet further embodiments, X₁ is N; and R₁ is hydrogen.

In yet further embodiments, Z is cyclohexyl, which may be optionally substituted with at least one substituent selected from the group consisting of lower alkyl, lower alkanoyl, lower heteroalkyl, lower alkoxy, oxo, lower acyloxy, carboxyl, lower carboxyester, and lower alkylamino.

In yet further embodiments, Z is cyclohexyl which is substituted in the 4-position with a substituent selected from the group consisting of lower alkyl and lower alkoxy; R₁₁ is hydrogen; and R₁₄ is methyl.

In yet further embodiments, Z is 4-alkylcyclohexyl.

In yet further embodiments, Z is 4-methylcyclohexyl.

In certain embodiments of the present invention, compounds have structural Formula VI:

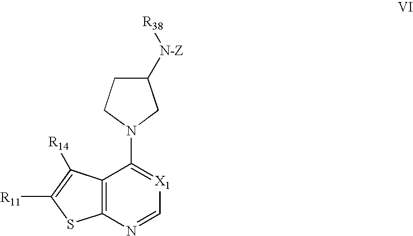

or a salt thereof, wherein:
X₁ is selected from the group consisting of [C(R₂)] and N;
Z is selected from the group consisting of hydrogen, aryl, alkyl, heterocycloalkyl, alkoxylcarbonyl, acyl, and cycloalkyl, any of which may be optionally substituted;
R₂, R₁₄, and R₃₄ are each independently selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted; R₁₁ is selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted; and
R₁₁ and R₁₄ may be joined together to form a partially saturated cycloalkyl.

In further embodiments, X₁ is N; and R₁₁ and R₁₄ are each independently selected from the group consisting of hydrogen and C₁-C₃ alkyl.

In yet further embodiments, R₁₁ is hydrogen; and R₁₄ is methyl.

In yet further embodiments, Z is selected from the group consisting of alkoxylcarbonyl and acyl; and R₃₄ is lower alkyl.

As used herein, the terms below have the meanings indicated.

When ranges of values are disclosed, and the notation “from n₁ . . . to n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycle, or any other moiety where the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon group having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—), (—C::C—)]. Examples of suitable alkenyl groups include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether group, wherein the term alkyl is as defined below. Examples of suitable alkyl ether groups include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl group containing from 1 to 20 carbon atoms. In certain embodiments, said alkyl group will comprise from 1 to 10 carbon atoms. In further embodiments, said alkyl group will comprise from 1 to 6 carbon atoms. Alkyl groups may be optionally substituted as defined herein. Examples of alkyl groups include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) group wherein the term alkyl is as defined above and wherein the sulfur may be singly or doubly oxidized. Examples of suitable alkyl thioether groups include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon group having one or more triple bonds and containing from 2 to 20 carbon atoms. In certain embodiments, said alkynyl group comprises from 2 to 6 carbon atoms. In further embodiments, said alkynyl group comprises from 2 to 4 carbon atoms. The term “alkynylene” refers to a carbon-carbon triple bond attached at two positions such as ethynylene (—C:::C—, —C≡C—). Examples of alkynyl groups include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)—NR₂ group with R as defined herein. The term “N-amido” as used herein, alone or in combination, refers to a RC(═O)NH— group, with R as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl group derived from an aryl-substituted alkanecarboxylic acid such as benzoyl, naphthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent group C₆H₄═ derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′ group, with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronaphthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl group having the meaning as defined above wherein one or more hydrogens are replaced with a halogen. Specifically embraced are monohaloalkyl, dihaloalkyl and polyhaloalkyl groups. A monohaloalkyl group, for one example, may have an iodo, bromo, chloro or fluoro atom within the group. Dihalo and polyhaloalkyl groups may have two or more of the same halo atoms or a combination of different halo groups. Examples of haloalkyl groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon group, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 7 membered unsaturated heteromonocyclic ring, or a fused monocyclic, bicyclic, or tricyclic ring system in which at least one of the fused rings is aromatic, which contains at least one atom selected from the group consisting of O, S, and N. In certain embodiments, said heteroaryl will comprise from 5 to 7 carbon atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic group containing at least one heteroatom as a ring member, wherein each said heteroatom may be independently selected from the group consisting of nitrogen, oxygen, and sulfur. In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms.

The term “lower aryl,” as used herein, alone or in combination, means phenyl or naphthyl, which may be optionally substituted as provided.

The term “lower heteroalkyl,” as used herein, alone or in combination, refers to a stable straight or branched chain, or cyclic hydrocarbon group, or combinations thereof, fully saturated or containing from 1 to 3 degrees of unsaturation, consisting of one to six atoms in which one to three may be heteroatoms selected from the group consisting of O, N, and S, and the remaining atoms are carbon. The nitrogen and sulfur atoms may optionally be oxidized and the nitrogen heteroatom may optionally be quaternized. The heteroatom(s) O, N and S may be placed at any interior or terminal position of the heteroalkyl group. Up to two heteroatoms may be consecutive, such as, for example, —CH₂—NH—OCH₃.

The term “lower heteroaryl,” as used herein, alone or in combination, means either 1) monocyclic heteroaryl comprising five or six ring members, of which between one and four said members may be heteroatoms selected from the group consisting of O, S, and N, or 2) bicyclic heteroaryl, wherein each of the fused rings comprises five or six ring members, comprising between them one to four heteroatoms selected from the group consisting of O, S, and N.

The term “lower cycloalkyl,” as used herein, alone or in combination, means a monocyclic cycloalkyl having between three and six ring members. Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms selected from the group consisting of O, S, and N. Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently selected from the group consisting of hydrogen and optionally substituted lower alkyl.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer to the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′ group with R and R′ as defined herein.

The term “thiocyanato” refers to a —CNS group.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, lower alkoxyalkyl, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “inhibition” (and by extension, “inhibitor”) as used herein encompasses all forms of functional protein (enzyme, kinase, receptor, channel, etc., for example) inhibition, including neutral antagonism, inverse agonism, competitive inhibition, and non-competitive inhibition (such as allosteric inhibition) Inhibition may be phrased in terms of an IC₅₀, defined below.

In certain embodiments, “H₁R inhibitor” is used herein to refer to a compound that exhibits an IC₅₀ with respect to the histamine type-1 receptor of no more than about 100 μM and more typically not more than about 50 μM, as measured in the in vitro histamine receptor cell-based assays described generally hereinbelow. Similarly, “H₄R inhibitor” is used herein to refer to a compound that exhibits an IC₅₀ with respect to the histamine type-4 receptor of no more than about 100 μM and more typically not more than about 50 μM, as measured in the in vitro histamine receptor cell-based assays described generally hereinbelow. A “H₁/H₄ inhibitor” is used herein to refer to a compound that exhibits an IC₅₀ with respect to both the histamine type-1 receptor and the histamine type-4 receptor of no more than about 100 μM and more typically not more than about 50 μM, as measured in the in vitro histamine receptor cell-based assays described generally hereinbelow; the amount of inhibition need not be equivalent at each receptor, but should not be negligible. In certain embodiments, such as, for example, in the case of an in vitro ligand-binding assay protocol, “IC₅₀” is that concentration of inhibitor which is required to displace a natural ligand or reference standard to a half-maximal level. In other embodiments, such as, for example, in the case of certain cellular or in vivo protocols which have a functional readout, “IC₅₀” is that concentration of inhibitor which reduces the activity of a functional protein (e.g., H₁R and/or H₄R) to a half-maximal level. Certain compounds disclosed herein have been discovered to exhibit inhibitory activity against H₁R and/or H₄R. In certain embodiments, compounds will exhibit an IC₅₀ with respect to H₁R and/or H₄R of no more than about 10 μM; in further embodiments, compounds will exhibit an IC₅₀ with respect to H₁R and/or H₄R of no more than about 5 μM; in yet further embodiments, compounds will exhibit an IC₅₀ with respect to H₁R and/or H₄R of not more than about 1 μM; in yet further embodiments, compounds will exhibit an IC₅₀ with respect to H₁R and/or H₄R of not more than about 200 nM, as measured in the H₁R and/or H₄R assay described herein.

The phrase “therapeutically effective” is intended to qualify the amount of active ingredients used in the treatment of a disease or disorder. This amount will achieve the goal of reducing or eliminating the said disease or disorder.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate(besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate(isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate(p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

While it may be possible for the compounds of the subject invention to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual, ocular, and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Examples of fillers or diluents for use in oral pharmaceutical formulations such as capsules and tablets include, without limitation, lactose, mannitol, xylitol, dextrose, sucrose, sorbitol, compressible sugar, microcrystalline cellulose (MCC), powdered cellulose, cornstarch, pregelatinized starch, dextrates, dextran, dextrin, dextrose, maltodextrin, calcium carbonate, dibasic calcium phosphate, tribasic calcium phosphate, calcium sulfate, magnesium carbonate, magnesium oxide, poloxamers such as polyethylene oxide, and hydroxypropyl methyl cellulose. Fillers may have complexed solvent molecules, such as in the case where the lactose used is lactose monohydrate. Fillers may also be proprietary, such in the case of the filler PROSOLV® (available from JRS Pharma). PROSOLV is a proprietary, optionally high-density, silicified microcrystalline cellulose composed of 98% microcrystalline cellulose and 2% colloidal silicon dioxide. Silicification of the microcrystalline cellulose is achieved by a patented process, resulting in an intimate association between the colloidal silicon dioxide and microcrystalline cellulose. ProSolv comes in different grades based on particle size, and is a white or almost white, fine or granular powder, practically insoluble in water, acetone, ethanol, toluene and dilute acids and in a 50 g/1 solution of sodium hydroxide.

Examples of disintegrants for use in oral pharmaceutical formulations such as capsules and tablets include, without limitation, sodium starch glycolate, sodium carboxymethyl cellulose, calcium carboxymethyl cellulose, croscarmellose sodium, povidone, crospovidone(polyvinylpolypyrrolidone), methyl cellulose, microcrystalline cellulose, powdered cellulose, low-substituted hydroxy propyl cellulose, starch, pregelatinized starch, and sodium alginate.

Additionally, glidants and lubricants may be used in oral pharmaceutical formulations to ensure an even blend of excipients upon mixing. Examples of lubricants include, without limitation, calcium stearate, glyceryl monostearate, glyceryl palmitostearate, hydrogenated vegetable oil, light mineral oil, magnesium stearate, mineral oil, polyethylene glycol, sodium benzoate, sodium lauryl sulfate, sodium stearyl fumarate, stearic acid, talc, and zinc stearate. Examples of glidants include, without limitation, silicon dioxide (SiO₂), talc cornstarch, and poloxamers. Poloxamers (or LUTROL®, available from the BASF Corporation) are A-B-A block copolymers in which the A segment is a hydrophilic polyethylene glycol homopolymer and the B segment is hydrophobic polypropylene glycol homopolymer.

Examples of tablet binders include, without limitation, acacia, alginic acid, carbomer, carboxymethyl cellulose sodium, dextrin, ethylcellulose, gelatin, guar gum, hydrogenated vegetable oil, hydroxyethyl cellulose, hydroxypropyl cellulose, hydroxypropylmethyl cellulose, copolyvidone, methyl cellulose, liquid glucose, maltodextrin, polymethacrylates, povidone, pregelatinized starch, sodium alginate, starch, sucrose, tragacanth, and zein.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

Topical ophthalmic, otic, and nasal formulations of the present invention may comprise excipients in addition to the active ingredient. Excipients commonly used in such formulations include, but are not limited to, tonicity agents, preservatives, chelating agents, buffering agents, and surfactants. Other excipients comprise solubilizing agents, stabilizing agents, comfort-enhancing agents, polymers, emollients, pH-adjusting agents and/or lubricants. Any of a variety of excipients may be used in formulations of the present invention including water, mixtures of water and water-miscible solvents, such as C1-C7-alkanols, vegetable oils or mineral oils comprising from 0.5 to 5% non-toxic water-soluble polymers, natural products, such as alginates, pectins, tragacanth, karaya gum, guar gum, xanthan gum, carrageenin, agar and acacia, starch derivatives, such as starch acetate and hydroxypropyl starch, and also other synthetic products such as polyvinyl alcohol, polyvinylpyrrolidone, polyvinyl methyl ether, polyethylene oxide, preferably cross-linked polyacrylic acid and mixtures of those products. The concentration of the excipient is, typically, from 1 to 100,000 times the concentration of the active ingredient. In preferred embodiments, the excipients to be included in the formulations are typically selected on the basis of their inertness towards the active ingredient component of the formulations.

Relative to ophthalmic, otic, and nasal formulations, suitable tonicity-adjusting agents include, but are not limited to, mannitol, sodium chloride, glycerin, sorbitol and the like. Suitable buffering agents include, but are not limited to, phosphates, borates, acetates and the like. Suitable surfactants include, but are not limited to, ionic and nonionic surfactants (though nonionic surfactants are preferred), RLM 100, POE 20 cetylstearyl ethers such as Procol® CS20 and poloxamers such as Pluronic® F68.

The formulations set forth herein may comprise one or more preservatives. Examples of such preservatives include p-hydroxybenzoic acid ester, sodium perborate, sodium chlorite, alcohols such as chlorobutanol, benzyl alcohol or phenyl ethanol, guanidine derivatives such as polyhexamethylene biguanide, sodium perborate, polyquaternium-1, amino alcohols such as AMP-95, or sorbic acid. In certain embodiments, the formulation may be self-preserved so that no preservation agent is required.

For ophthalmic, otic, or nasal administration, the formulation may be a solution, a suspension, or a gel. In preferred aspects, the formulations are for topical application to the eye, nose, or ear in aqueous solution in the form of drops. The term “aqueous” typically denotes an aqueous formulation wherein the formulation is >50%, more preferably >75% and in particular >90% by weight water. These drops may be delivered from a single dose ampoule which may preferably be sterile and thus render bacteriostatic components of the formulation unnecessary. Alternatively, the drops may be delivered from a multi-dose bottle which may preferably comprise a device which extracts any preservative from the formulation as it is delivered, such devices being known in the art.

For ophthalmic disorders, components of the invention may be delivered to the eye as a concentrated gel or a similar vehicle, or as dissolvable inserts that are placed beneath the eyelids.

The formulations of the present invention that are adapted for topical administration to the eye are preferably isotonic, or slightly hypotonic in order to combat any hypertonicity of tears caused by evaporation and/or disease. This may require a tonicity agent to bring the osmolality of the formulation to a level at or near 210-320 milliosmoles per kilogram (mOsm/kg). The formulations of the present invention generally have an osmolality in the range of 220-320 mOsm/kg, and preferably have an osmolality in the range of 235-300 mOsm/kg. The ophthalmic formulations will generally be formulated as sterile aqueous solutions.

In certain ophthalmic embodiments, the compositions of the present invention are formulated with one or more tear substitutes. A variety of tear substitutes are known in the art and include, but are not limited to: monomeric polyols, such as, glycerol, propylene glycol, and ethylene glycol; polymeric polyols such as polyethylene glycol; cellulose esters such hydroxypropylmethyl cellulose, carboxy methylcellulose sodium and hydroxy propylcellulose; dextrans such as dextran 70; vinyl polymers, such as polyvinyl alcohol; and carbomers, such as carbomer 934P, carbomer 941, carbomer 940 and carbomer 974P. Certain formulations of the present invention may be used with contact lenses or other ophthalmic products.

Preferred formulations are prepared using a buffering system that maintains the formulation at a pH of about 4.5 to a pH of about 8. A most preferred formulation pH is from 7 to 8.

In particular embodiments, a formulation of the present invention is administered once a day. However, the formulations may also be formulated for administration at any frequency of administration, including once a week, once every 5 days, once every 3 days, once every 2 days, twice a day, three times a day, four times a day, five times a day, six times a day, eight times a day, every hour, or any greater frequency. Such dosing frequency is also maintained for a varying duration of time depending on the therapeutic regimen. The duration of a particular therapeutic regimen may vary from one-time dosing to a regimen that extends for months or years. The formulations are administered at varying dosages, but typical dosages are one to two drops at each administration, or a comparable amount of a gel or other formulation. One of ordinary skill in the art would be familiar with determining a therapeutic regimen for a specific indication.

Gels for topical or transdermal administration may comprise, generally, a mixture of volatile solvents, nonvolatile solvents, and water. In certain embodiments, the volatile solvent component of the buffered solvent system may include lower (C1-C6) alkyl alcohols, lower alkyl glycols and lower glycol polymers. In further embodiments, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. In certain embodiments, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess may result in a lack of bioavailability due to poor release of drug from solvent mixture. The buffer component of the buffered solvent system may be selected from any buffer commonly used in the art; in certain embodiments, water is used. A common ratio of ingredients is about 20% of the nonvolatile solvent, about 40% of the volatile solvent, and about 40% water. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, galactomannan polymers (such as guar and derivatives thereof) and cosmetic agents.

Lotions include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as stearic or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops may comprise sterile aqueous or oily solutions or suspensions and may be prepared by dissolving the active ingredient in a suitable aqueous solution of a bactericidal and/or fungicidal agent and/or any other suitable preservative, and, in certain embodiments, including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Non-limiting examples of possible combination therapies include use of certain compounds of the invention with H₁R antagonists and/or H₃R antagonists. Specific, non-limiting examples of possible combination therapies include use of certain compounds of the invention with H₁R antagonists such as acrivastine, alcaftadine, antazoline, azelastine, bromazine, brompheniramine, cetirizine, chlorpheniramine, clemastine, desloratidine, diphenhydramine, diphenylpyraline, ebastine, emedastine, epinastine, fexofenadine, hydroxyzine, ketotifen, levocabastine, levocetirizine, loratidine, methdilazine, mizolastine, promethazine, olopatadine, and triprolidine.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, certain embodiments provide methods for treating H₁R and/or H₄R-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of H₁R and/or H₄R-mediated disorders. Specific diseases to be treated by the compounds, compositions, and methods disclosed herein include inflammation and related diseases, including autoimmune diseases. The compounds are useful to treat arthritis, including but not limited to rheumatoid arthritis, spondyloarthropathies, gouty arthritis, osteoarthritis, systemic lupus erythematosus, juvenile arthritis, acute rheumatic arthritis, enteropathic arthritis, neuropathic arthritis, psoriatic arthritis, and pyogenic arthritis. The compounds are also useful in treating osteoporosis and other related bone disorders. These compounds can also be used to treat gastrointestinal conditions such as reflux esophagitis, diarrhea, inflammatory bowel disease, Crohn's disease, gastritis, irritable bowel syndrome and ulcerative colitis. The compounds may also be used in the treatment of upper respiratory inflammation, such as, but not limited to, seasonal allergic rhinitis, non-seasonal allergic rhinitis, acute non-allergic rhinitis, chronic non-allergic rhinitis, Sampter's triad, non-allergic rhinitis with eosinophilia syndrome, nasal polyposis, atrophic rhinitis, hypertrophic rhinitis, membranous rhinitis, vasomotor rhinitis, rhinosinusitis, chronic rhinopharyngitis, rhinorrhea, occupational rhinitis, hormonal rhinitis, drug-induced rhinitis, gustatory rhinitis, as well as pulmonary inflammation, such as that associated with viral infections and cystic fibrosis. In addition, compounds disclosed herein are also useful in organ transplant patients either alone or in combination with conventional immunomodulators.

Moreover, compounds disclosed herein may be used in the treatment of tendonitis, bursitis, skin-related conditions such as psoriasis, allergic dermatitis, atopic dermatitis and other variants of eczema, allergic contact dermatitis, irritant contact dermatitis, seborrhoeic eczema, nummular eczematous dermatitis, autosensitization dermatitis, Lichen Simplex Chronicus, dyshidrotic dermatitis, neurodermatitis, stasis dermatitis, generalized ordinary urticaria, acute allergic urticaria, chronic allergic urticaria, autoimmune urticaria, chronic idiopathic urticaria, drug-induced urticaria, cholinergic urticaria, chronic cold urticaria, dermatographic urticaria, solar urticaria, urticaria pigmentosa, mastocytosis, acute or chronic pruritis associated with skin-localized or systemic diseases and disorders, such as pancreatitis, hepatitis, burns, sunburn, and vitiligo.

Further, the compounds disclosed herein can be used to treat respiratory diseases, including therapeutic methods of use in medicine for preventing and treating a respiratory disease or condition including: asthmatic conditions including allergen-induced asthma, exercise-induced asthma, pollution-induced asthma, cold-induced asthma, and viral-induced-asthma; chronic obstructive pulmonary diseases including chronic bronchitis with normal airflow, chronic bronchitis with airway obstruction (chronic obstructive bronchitis), emphysema, asthmatic bronchitis, and bullous disease; and other pulmonary diseases involving inflammation including bronchioectasis cystic fibrosis, pigeon fancier's disease, farmer's lung, acute respiratory distress syndrome, pneumonia, aspiration or inhalation injury, fat embolism in the lung, acidosis inflammation of the lung, acute pulmonary edema, acute mountain sickness, acute pulmonary hypertension, persistent pulmonary hypertension of the newborn, perinatal aspiration syndrome, hyaline membrane disease, acute pulmonary thromboembolism, heparin-protamine reactions, sepsis, status asthamticus and hypoxia.

The compounds disclosed herein are also useful in treating tissue damage in such diseases as vascular diseases, periarteritis nodosa, thyroiditis, sclerodoma, rheumatic fever, type I diabetes, neuromuscular junction disease including myasthenia gravis, white matter disease including multiple sclerosis, sarcoidosis, nephritis, nephrotic syndrome, Behcet's syndrome, polymyositis, gingivitis, periodontis, hypersensitivity, and swelling occurring after injury.

The compounds disclosed herein can be used in the treatment of otic diseases and otic allergic disorders, including eustachian tube itching.

The compounds disclosed herein can be used in the treatment of ophthalmic diseases, such as ophthalmic allergic disorders, including allergic conjunctivitis, vernal conjunctivitis, vernal keratoconjunctivitis, and giant papillary conjunctivitis, dry eye, glaucoma, glaucomatous retinopathy, diabetic retinopathy, retinal ganglion degeneration, ocular ischemia, retinitis, retinopathies, uveitis, ocular photophobia, and of inflammation and pain associated with acute injury to the eye tissue. The compounds can also be used to treat post-operative inflammation or pain as from ophthalmic surgery such as cataract surgery and refractive surgery. In preferred embodiments, the compounds of the present invention are used to treat an allergic eye disease selected from the group consisting of allergic conjunctivitis; vernal conjunctivitis; vernal keratoconjunctivitis; and giant papillary conjunctivitis.

Compounds disclosed herein are useful in treating patients with inflammatory pain such as reflex sympathetic dystrophy/causalgia (nerve injury), peripheral neuropathy (including diabetic neuropathy), and entrapment neuropathy (carpel tunnel syndrome). The compounds are also useful in the treatment of pain associated with acute herpes zoster (shingles), postherpetic neuralgia (PHN), and associated pain syndromes such as ocular pain. Pain indications include, but are not limited to, pain resulting from dermal injuriesand pain-related disorders such as tactile allodynia and hyperalgesia. The pain may be somatogenic (either nociceptive or neuropathic), acute and/or chronic.

The present compounds may also be used in co-therapies, partially or completely, in place of other conventional anti-inflammatory therapies, such as together with steroids, NSAIDs, COX-2 selective inhibitors, 5-lipoxygenase inhibitors,

LTB₄ antagonists and LTA₄ hydrolase inhibitors. The compounds disclosed herein may also be used to prevent tissue damage when therapeutically combined with antibacterial or antiviral agents.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

All references, patents or applications, U.S. or foreign, cited in the application are hereby incorporated by reference as if written herein in their entireties. Where any inconsistencies arise, material literally disclosed herein controls.

The invention is further illustrated by the following examples, which may be made my methods known in the art. Additionally, these compounds may be commercially available.

### EXAMPLE 1

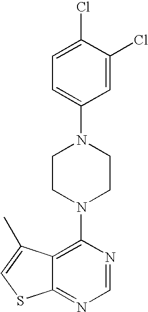

### 4-(4-(3,4-dichlorophenyl)piperazin-1-yl)-5-methylthieno[2,3-d]pyrimidine

The title compound was obtained commercially.

### EXAMPLE 2

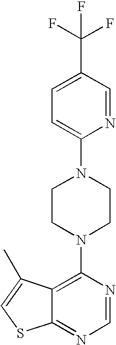

### 5-methyl-4-(4-(5-(trifluoromethyl)pyridin-2-yl)piperazin-1-yl)thieno[2,3-d]pyrimidine

The title compound was obtained commercially.

### EXAMPLE 3

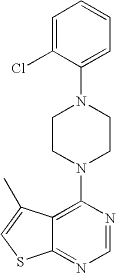

### 4-(4-(2-chlorophenyl)piperazin-1-yl)-5-methylthieno[2,3-d]pyrimidine

The title compound was obtained commercially.

### EXAMPLE 4

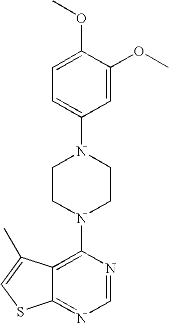

### 4-(4-(3,4-dimethoxyphenyl)piperazin-1-yl)-5-methylthieno[2,3-d]pyrimidine

The title compound was obtained commercially.

### EXAMPLE 5

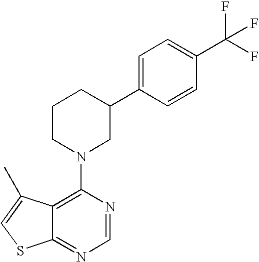

### 5-methyl-4-(3-(4-(trifluoromethyl)phenyl)piperidin-1-yl)thieno[2,3-d]pyrimidine

The title compound was obtained commercially.

### EXAMPLE 6

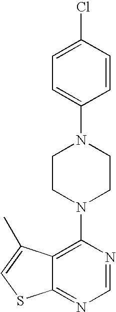

### 4-(4-(4-chlorophenyl)piperazin-1-yl)-5-methylthieno[2,3-d]pyrimidine

The title compound was obtained commercially.

### EXAMPLE 7

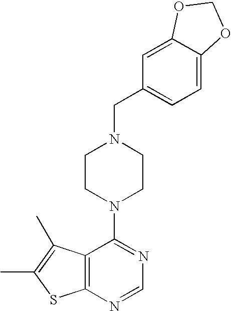

### 4-(4-(benzo[d][1,3]dioxol-5-ylmethyl)piperazin-1-yl)-5,6-dimethylthieno[2,3-d]pyrimidine

The title compound was obtained commercially.

### EXAMPLE 8

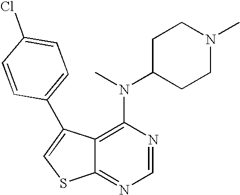

### 5-(4-chlorophenyl)-N-methyl-N-(1-methylpiperidin-4-yl)thieno[2,3-d]pyrimidin-4-amine

The title compound was obtained commercially.

### EXAMPLE 9

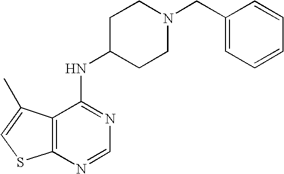

### N-(1-benzylpiperidin-4-yl)-5-methylthieno[2,3-d]pyrimidin-4-amine

The title compound was obtained commercially.

### EXAMPLE 10

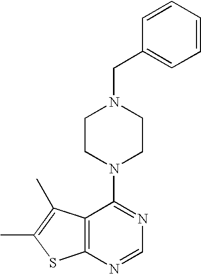

### 4-(4-benzylpiperazin-1-yl)-5,6-dimethylthieno[2,3-d]pyrimidine

The title compound was obtained commercially.

### EXAMPLE 11

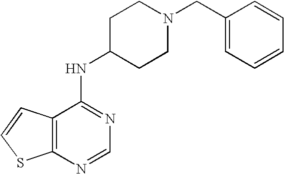

### N-(1-benzylpiperidin-4-yl)thieno[2,3-d]pyrimidin-4-amine

The title compound was obtained commercially.

### EXAMPLE 12

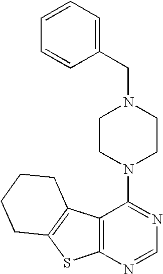

### 4-(4-benzylpiperazin-1-yl)-5,6-tetrahydrobenzo[b]thiophenethieno[2,3-d]pyrimidine

The title compound was obtained commercially.

### EXAMPLE 13

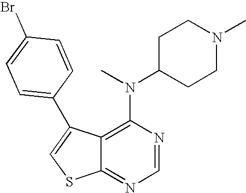

### 5-(4-bromophenyl)-N-methyl-N-(1-methylpiperidin-4-yl)thieno[2,3-d]pyrimidin-4-amine

The title compound was obtained commercially.

### EXAMPLE 14

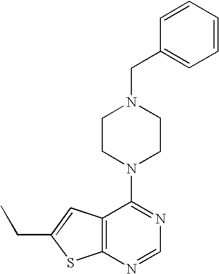

### 4-(4-benzylpiperazin-1-yl)-6-ethylthieno[2,3-d]pyrimidine

The title compound was obtained commercially.

### EXAMPLE 15

This example has intentionally been left blank

### EXAMPLE 16

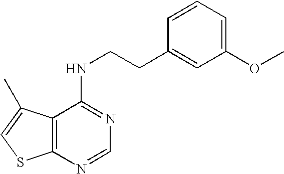

### [2-(3-Methoxyphenyl)ethyl](5-methylthiopheno[3,2-e]pyrimidin-4-yl)amine

A 50 mL round bottom flask was charged with 4-chloro-5-methylthiopheno[2,3-d]pyrimidine (0.30 g, 1.6 mmol), 2-(3-methoxyphenyl)ethylamine (0.30 mL, 2.0 mmol), triethylamine (0.45 mL, 3.2 mmol) and EtOH (10 mL). The resulting solution was stirred at reflux for 4 h and then concentrated in vacuo. The residue was purified by flash column chromatography on silica gel eluting with 10% ethyl acetate in petroleum ether, to afford 0.33 g (68%) of the product as white solid. ¹H NMR (300 MHz, CDCl₃) δ: 8.45 (s, 1H), 7.29-7.22 (m, 1H), 6.86-6.76 (m, 4H), 5.40 (br, 1H), 3.89 (m, 2H), 3.78 (s, 3H), 2.98 (t, J=6.6 Hz, 2H), 2.33 (d, J=1.2 Hz, 3H). MS m/z 300 (M+H⁺).

### EXAMPLE 17

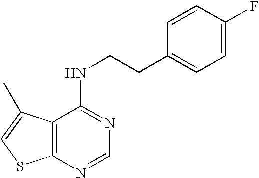

### (4-Fluorophenyl)[2-(5-methylthiopheno[3,2-e]pyrimidin-4-yl)ethyl]amine

The title compound was prepared as described in Example 16, except that 2-(4-fluorophenyl)ethylamine was substituted for 2-(3-methoxyphenyl)ethylamine. ¹H NMR (300 MHz, CDCl₃) δ: 8.45 (s, 1H), 7.20 (m, 2H), 7.02 (m, 2H), 6.74 (q, J=1.2 Hz, 1H), 5.38 (br, 1H), 3.86 (m, 2H), 2.98 (t, J=6.6 Hz, 2H), 2.35 (d, J=1.2 Hz, 3H). MS m/z: 288 (M+H⁺).

### EXAMPLE 18

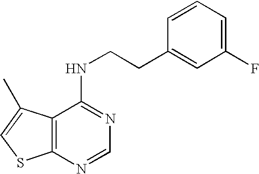

### (3-Fluorophenyl)[2-(5-methylthiopheno[3,2-e]pyrimidin-4-yl)ethyl]amine

The title compound was prepared as described in Example 16, except that 2-(3-fluorophenyl)ethylamine was substituted for 2-(3-methoxyphenyl)ethylamine. ¹H NMR (300 MHz, CDCl₃) δ: 8.46 (s, 1H), 7.30 (m, 1H), 6.98 (m, 3H), 6.79 (q, J=1.2 Hz, 1H), 5.39 (br, 1H), 3.88 (m, 2H), 3.00 (t, J=6.9 Hz, 2H), 2.36 (d, J=1.2 Hz, 3H). MS m/z: 288 (M+H⁺).

### EXAMPLE 19

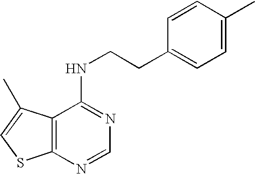

### (4-Methylphenyl)[2-(5-methylthiopheno[3,2-e]pyrimidin-4-yl)ethyl]amine

The title compound was prepared as described in Example 16, except that 2-(4-methylphenyl)ethylamine was substituted for 2-(3-methoxyphenyl)ethylamine. ¹H NMR (300 MHz, CDCl₃) δ: 8.45 (s, 1H), 7.18 (m, 4H), 6.77 (q, J=1.2 Hz, 1H), 5.38 (br, 1H), 3.87 (m, 2H), 2.96 (t, J=6.9 Hz, 2H), 2.36 (s, 3H), 2.33 (d, J=1.2 Hz, 3H). MS m/z: 284 (M+H⁺).

### EXAMPLE 20

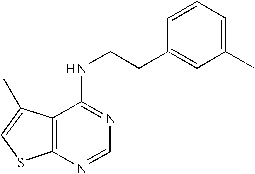

### (3-Methylphenyl)[2-(5-methylthiopheno[3,2-e]pyrimidin-4-yl)ethyl]amine

The title compound was prepared as described in Example 16, except that 2-(3-methylphenyl)ethylamine was substituted for 2-(3-methoxyphenyl)ethylamine. ¹H NMR (300 MHz, CDCl₃) δ: 8.45 (s, 1H), 7.22 (m, 1H), 7.06 (m, 3H), 6.77 (q, J=1.2 Hz, 1H), 5.39 (br, 1H), 3.87 (m, 2H), 2.96 (t, J=6.6 Hz, 2H), 2.34 (s, 3H), 2.32 (d, J=1.2 Hz, 3H). MS m/z: 284 (M+H⁺).

### EXAMPLE 21

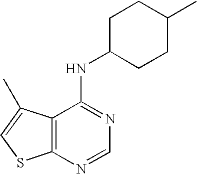

### (4-Methylcyclohexyl)(5-methylthiopheno[3,2-e]pyrimidin-4-yl)amine

The title compound was prepared as described in Example 16, except that 4-methylcyclohexylamine was substituted for 2-(3-methoxyphenyl)ethylamine. ¹H NMR (300 MHz, CD₃OD) δ: 8.62 (s, 1H), 7.37 (m, 1H), 4.27 (m, 1H), 2.69 (d, J=1.2 Hz, 3H), 2.12-1.11 (m, 10H), 0.96 (d, J=6.6 Hz, 3H). MS m/z: 262 (M+H⁺).

### EXAMPLE 22

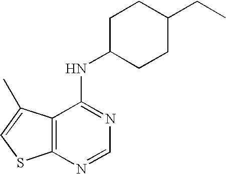

### (4-Ethylcyclohexyl)(5-methylthiopheno[3,2-e]pyrimidin-4-yl)amine

The title compound was prepared as described in Example 16, except that 4-ethylcyclohexylamine was substituted for 2-(3-methoxyphenyl)ethylamine. ¹H NMR (300 MHz, CD₃OD) δ: 8.61 (s, 1H), 7.36 (m, 1H), 4.28 (m, 1H), 2.73 (d, J=1.2 Hz, 3H), 2.15-1.08 (m, 12H), 0.95 (t, J=7.2 Hz, 3H). MS m/z: 276 (M+H⁺).

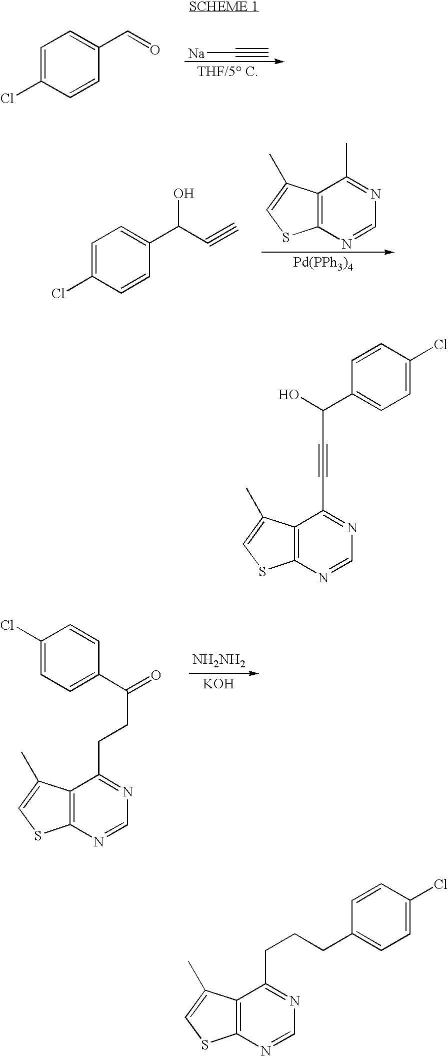

### EXAMPLE 23

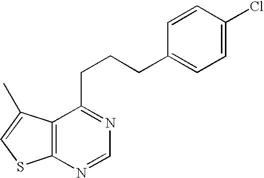

### 4-(3-(4-chlorophenyl)propyl)-5-methylthieno[2,3-d]pyrimidine

### EXAMPLE 24

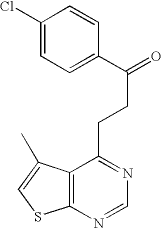

### 4-(3-(4-chlorophenyl)propyl)-5-methylthieno[2,3-d]pyrimidine

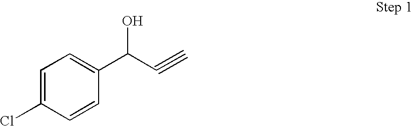

### 1-(4-chlorophenyl)prop-2-yn-1-ol

A 50-mL round-bottom flask under nitrogen, was charged with a solution of 4-chlorobenzaldehyde (280 mg, 2.00 mmol) in THF (10 mL). To the mixture was added ethynylsodium (105 mg, 2.21 mmol). The resulting solution was stirred for 1 hr at 0° C. Reaction progress was monitored by TLC (ethyl acetate/petroleum ether=1:2). The reaction was quenched by the addition of 10 mL of water, extracted with 3×20 mL of ethyl acetate, dried over anhydrous sodium sulfate, filtered and purified by silica gel column chromatography eluted with ethyl acetate/petroleum ether (1:10). This gave 400 mg (120%) of crude product as a yellow oil.

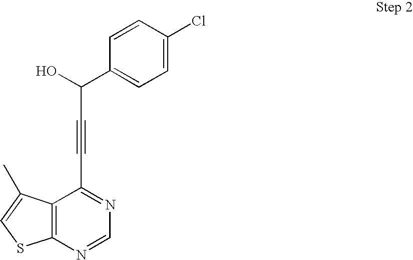

### 1-(4-chlorophenyl)-3-(5-methylthieno[2,3-d]pyrimidin-4-yl)prop-2-yn-1-ol

A 100-mL round-bottom flask under nitrogen, was charged with 4-iodo-5-methylthieno[2,3-d]pyrimidine (1.9 g, 6.82 mmol, prepared as described in Bulletin de la Societe Chimique de France (1975), (3-4, Pt. 2), 592-7), and DMF (60 mL). To this solution was added 1-(4-chlorophenyl)prop-2-yn-1-ol (2.3 g, 13.72 mmol), Pd(PPh₃)₂Cl₂ (387 mg, 0.55 mmol), and CuI (157 mg, 0.82 mmol). To the mixture was added TEA (2.8 g, 27.45 mmol). The resulting solution was stirred for 5 hours at room temperature. A precipitate formed that was collected by filtration, resulting in 1.5 g (67%) of crude product as a yellow solid.

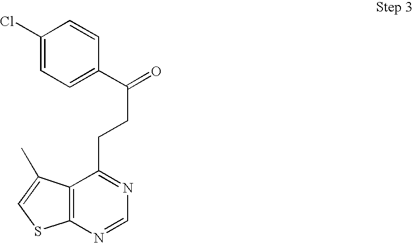

### 1-(4-chlorophenyl)-3-(5-methylthieno[2,3-d]pyrimidin-4-yl)propan-1-one

A 50-mL round-bottom flask under nitrogen was charged with 1-(4-chlorophenyl)-3-(5-methylthieno[2,3-d]pyrimidin-4-yl)prop-2-yn-1-ol (240 mg, 0.76 mmol), CH₂Cl₂ (30 mL), Et₃SiH (177 mg, 1.51 mmol), and TFA (784 mg, 6.81 mmol). The resulting solution was stirred for 5 hr at room temperature. The reaction was quenched by addition of 20 mL of NaHCO₃/H₂O. The resulting solution was extracted with 3×30 mL of CH₂Cl₂. The organic layers were combined, dried over Na₂SO₄, concentrated and purified by silica gel chromatography with petroleum ether/Ethyl acetate (5:1). This resulted in 150 mg (56%) of product as a white solid. ¹H NMR (300 MHz, CDCl₃) δ: 2.76 (3H, s), 3.65 (2H, dd. J=8.4), 3.73 (2H, dd, J=8.4), 7.17 (1H, s), 7.47 (2H, d, J=8.4), 8.00 (2H ,d, J=8.4), 8.87 (1H, s). LCMS: 317.8 (M+1)⁺.

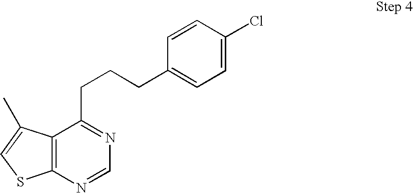

### 2-[3-(4-Fluoro-phenyl)-5-piperidin-4-yl-1H-pyrazol-4-yl]-thiazole-4-carboxyl acid(pyridin-3-ylmethyl)-amide

A 10-ml sealed tube was charged with 1-(4-chlorophenyl)-3-(5-methylthieno[2,3-d]pyrimidin-4-yl)propan-1-one (100 mg, 0.31 mmol), ethylene glycol (2 ml), and NH₂NH₂ (200 mg) at 0° C. To the solution was added KOH/H₂O (200 mg, 80%) in several aliquots at 0° C. The resulting solution was stirred for 2 hours at 180° C. The reaction was quenched by the addition of 10 ml of H₂O. The mixture was extracted with 3×30 ml of Ethyl acetate, dried over Na₂SO₄, and concentrated under vacuum. The residue was purified silica gel chromatography eluting with CH₂Cl₂:MeOH (100:1), resulting in 40 mg (38%) of 4product as a white solid. ¹H NMR (300 MHz, CDCl₃) δ: 2.14 (2H, m, J=7.8), 2.51 (3H, s), 2.77 (2H, t, J=7.5), 3.24 (2H, t, J=8.1) 7.15 (2H, d, J=7.8), 7.26 (1H, s), 7.28 (4H, m, J=6.3), 8.97 (1H, s). LCMS: 303.8 (M+1)⁺.

### EXAMPLE 25

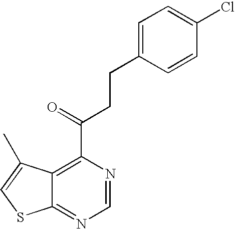

### 3-(4-chlorophenyl)-1-(5-methylthieno[2,3-d]pyrimidin-4-yl)propan-1-one

A 100-mL round-bottom flask under nitrogen, was charged with 4-bromo-5-methylthieno[2,3-d]pyrimidine (100 mg, 0.44 mmol, described in Bulletin de la Societe Chimique de France (1975), (3-4, Pt. 2), 592-7) in THF (50 mL, dry). To this solution at −78° C. was added n-BuLi (0.2 mL, 1.20 equiv). After 5 min 3-(4-chlorophenyl)-N-methoxy-N-methylpropanamide (110 mg, 0.48 mmol, 1.10 equiv) was added. The resulting solution was for 30 minutes while the temperature was slowly brought to at room temperature. The reaction was quenched by the addition of 30 mL of NH₄Cl (aq). The resulting solution was extracted with 2×100 mL of ethyl acetate. The organic layers combined, dried over anhydrous magnesium sulfate, filtered, concentrated, and purified by column chromatography, eluting with ethyl acetate/petroleum ether (1/10). This gave the product in 41 mg (30%) ¹H NMR (300 MHz, CDCl₃) δ: 2.14 (2H, m, J=7.8), 2.51 (3H, s), 2.77 (2H, t, J=7.5), 2.26 (3H, s), 3.11 (2H, t, J=7.2), 3.54 (2H, t, J=7.2),7.26 (5H, m), 9.09 (1H, s). LCMS: 317.1 (M+1)⁺.

### EXAMPLE 26

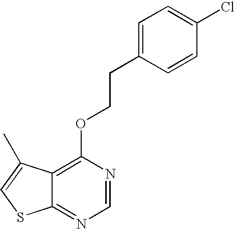

### 4-(4-chlorophenethoxy)-5-methylthieno[2,3-d]pyrimidine

A 50-mL round-bottom flask was charged with 2-(4-chlorophenyl)ethanol (200 mg, 1.28 mmol) and THF (20 mL, dry), under nitrogen. To the above was added NaH (80 mg, 3.33 mmol) in several batches over 5 min, followed by 4-bromo-5-methylthieno[2,3-d]pyrimidine (200 mg, 0.88 mmol). The resulting solution was stirred for 3 hr at reflux. The reaction was then quenched with 50 mL of water and extracted with 3×100 mL of ethyl acetate. The combined organic layers were dried over anhydrous magnesium sulfate, concentrated, and purified by column chromatography, eluting with ethyl acetate/petroleum ether (1:5). This gave in 200 mg (74%) of the desired product. ¹H NMR (300 MHz, CDCl₃) δ: 2.14 (2H, m, J=7.8), 2.51 (3H, s), 2.77 (2H, t, J=7.5), 2.26 (3H, s), 3.11 (2H, t, J=7.2), 3.54 (2H, t, J=7.2), 7.26 (5H, m), 9.09 (1H, s). LCMS: 317.1 (M+1)⁻.

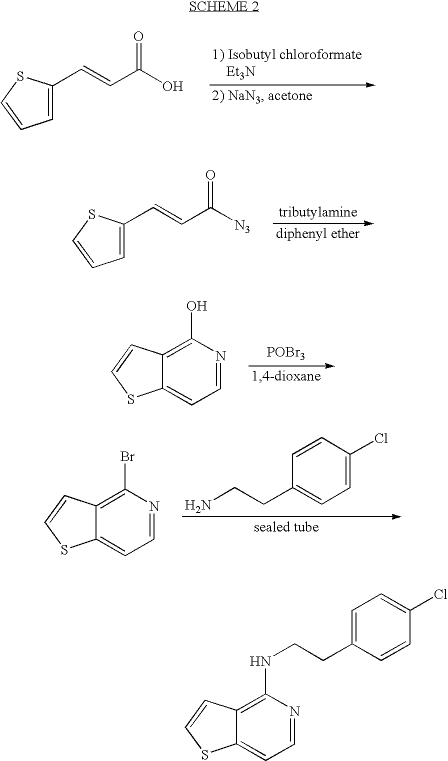

### EXAMPLE 27

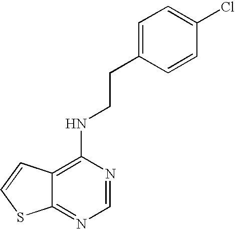

### N-(4-chlorophenethyl)thieno[3,2-c]pyridin-4-amine

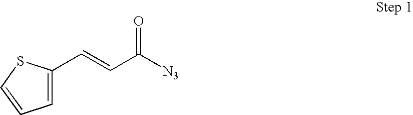

### (E)-3-(thiophen-2-yl)acryloyl azide

A 250 ml round bottom flask was charged with (E)-3-(thiophen-2-yl)acrylic acid (2.2 g, 14.27 mmol),acetone (60 mL), and Et₃N (1.47 g, 14.53 mmol). The resulting solution was cooled to 0° C. where isobutyl chloroformate (2.15 g, 15.74 mmol) was added drop wise. After stirring for 2 hours a solution of NaN₃ (1.37 g, 21.07 mmol) in H₂O (7 mL) was added. The resulting mixture was stirred at 0° C. for 1.5 hours. After this, the reaction mixture was poured into H₂O. The resulting precipitate was collected by filtration giving 2.2 g (78%) of product as a white solid after drying.

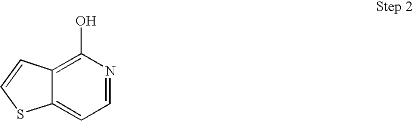

### thieno[3,2-c]pyridin-4-ol

A 250-mL round-bottom flask was charged with diphenyl ether (45 mL), tributylamine (2.47 g, 13.33 mmol) and heated to 190° C. To this solution was added drop wise a solution of (E)-1-azido-3-(thiophen-2-yl)prop-2-en-1-one (2.0 g, 11.16 mmol) in diphenyl ether (32 mL). The reaction mixture was stirred at 190° C. for 2.5 hours, cooled, and poured into petroleum ether (400 mL), and cooled in ice-bath. A solid formed which was filtered and washed with petroleum ether, resulting in 1.23 g (73%) of product as a gray solid.

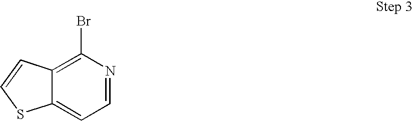

### 4-bromothieno[3,2-c]pyridine

A 100 mL round-bottom flask was charged with thieno[3,2-c]pyridin-4(5H)-one (300 mg, 1.98 mmol), dioxane (30 mL) and POBr₃ (1500 mg, 5.23 mmol), then stirred at 90° C. for 2 hours, and finally reflux for 1 hour. After cooling, ice-water was added to the mixture, and it was stirred for 15 minutes, and then neutralized with aqueous NaHCO₃. The mixture was extracted with CH₂Cl₂, dried over MgSO₄, filtered, concentrated, and purified by column chromatography with CH₂Cl₂/petroleum ether 2:1 giving 0.3 g (71%) of product as a white solid.

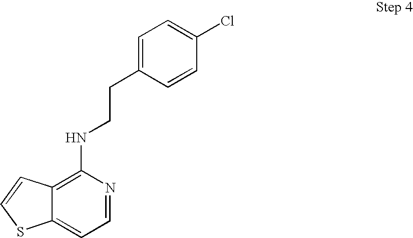

### N-(4-chlorophenethyl)thieno[3,2-c]pyridin-4-amine

A sealed tube was charged with 4-bromothieno[3,2-c]pyridine (100 mg, 0.47 mmol), and 2-(4-chlorophenyl)ethanamine (1.6 g, 10.28 mmol), then heated at 140° C. for 3 hours. The reaction was monitored by TLC (CH₂Cl₂:MeOH=40:1). After cooling, the mixture was purified by column chromatography with CH₂Cl₂/petroleum ether=3:1, giving 0.11 g (82%) of product as a pale yellow solid. ¹H NMR (300 MHz, DMSO-d6) δ: 2.94 (t, 2H, J=7.2 Hz), 3.66 (m, 2H), 7.14 (d, 1H, J=5.7 Hz), 7.22 (br, 1H), 7.29 (d, 2H, J=8.4 Hz), 7.36 (d, 2H, J=8.4 Hz), 7.59 (d, 1H, J=5.4 Hz), 7.68 (d, 1H, J=5.7 Hz), 7.86 (d, 1H, J=5.7 Hz) LCMS: 288 (M+1)⁺.

### EXAMPLE 28

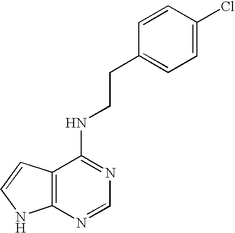

### N-(4-chlorophenethyl)-7H-pyrrolo[2,3-d]pyrimidin-4-amine

A 50-mL round-bottom under nitrogen, was was charged with 4-chloro-7H-pyrrolo[2,3-d]pyrimidine (50 mg, 0.33 mmol), 2-(4-chlorophenyl)ethanamine (150 mg, 0.97 mmol), and EtOH (20 mL). The resulting solution stirred for 24 hours at 110° C. Reaction progress was monitored by TLC (ethyl acetate: petroleum ether=1:2). When all starting material was consumed, the resulting mixture was concentrated under vacuum and purified by column chromatograph with ethyl acetate/petroleum ether (1:1). This gave 30 mg (34%) of the desired product. ¹H NMR (300 MHz, CDCl₃) δ: 2.90 (2H, m), 3.64 (2H, m), 6.52 (1H, s), 7.06 (1H, s), 7.27 (3H, m), 7.48 (1H, s), 8.12 (1H, s). LCMS: 273 (M+1)⁺.

### EXAMPLE 29

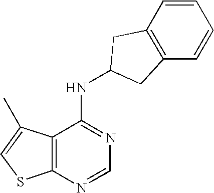

### N-(2,3-dihydro-1H-inden-2-yl)-5-methylthieno[2,3-d]pyrimidin-4-amine

A 100-mL round-bottom flask was charged with 2,3-dihydro-1H-inden-2-amine (200 mg, 1.50 mmol), 4-chloro-5-methylthieno[2,3-d]pyrimidine (100 mg, 0.44 mmol), Et₃N (100 mg, 0.99 mmol), and EtOH (50 mL). The resulting solution was stirred overnight at reflux. Reaction progress was monitored by TLC (ethyl acetate/petroleum ether=1:1). The mixture concentrated and purified by column chromatography, eluting with ethyl acetate/petroleum ether (1:20). This resulted in 46 mg (11%) of the desired product. Alternatively, the reaction could be run in DMF and heated via microwave at 150° C. for 10 min. ¹H NMR (300 MHz, CDCl₃) δ: 2.49 (3H, s), 2.98 (2H, m, J=3.6), 3.55 (2H, m, J=6.6), 5.14 (1H, s), 5.668(1H, s), 6.83 (1H, s), 7.24 (2H, s),7.28 (2H, s), 8.50 (1H, s). LCMS: 282.1 (M+1)⁺.

### EXAMPLE 30

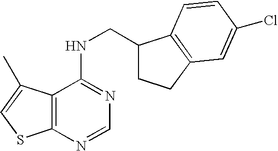

### N-((5-chloro-2,3-dihydro-1H-inden-1-yl)methyl)-5-methylthieno[2,3-d]pyrimidin-4-amine

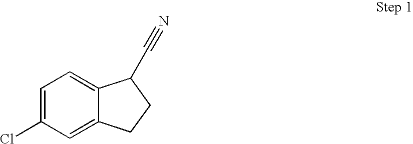

### 5-chloro-2,3-dihydro-1H-indene-1-carbonitrile

Sodium metal (2.1 g, 91.30 mmol) was dissolved in a mixture of EtOH (50 ml) and DME (100 ml), under nitrogen. This solution was added drop wise to a solution of 5-chloro-2,3-dihydroinden-1-one (5 g, 30.12 mmol) in DME (150 mL), which was then stirred under a hydrogen atmosphere. To this solution was added 1-(isocyanomethylsulfonyl)-4-methylbenzene (8.8 g, 45.13 mmol) at −5° C., which was stirred overnight at room temperature. Reaction progress was monitored by TLC (ethyl acetate/petroleum ether=1:2). The reaction was then quenched by the addition of 100 ml of water, and then extracted with 3×200 mL of ethyl acetate. The organics were combined, dried over anhydrous magnesium sulfate, concentrated, and purified by silica gel chromatography with ethyl acetate/petroleum ether (1:10). This gave 3.7 g (69%) of the product as a yellow oil.

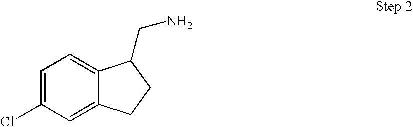

### (5-chloro-2,3-dihydro-1H-inden-1-yl)methanamine

A 100-mL round-bottom flask was charged with 5-chloro-2,3-dihydro-1H-indene-1-carbonitrile (200 mg, 1.13 mmol, 1.00 equiv) and THF (30 mL). To this solution was added BH₃-THF (3 mL). The reaction was stirred for 2 hours at reflux. Reaction progress was monitored by TLC (ethyl acetate/petroleum ether=1:1). The reaction was quenched by adjusting the pH to 1 with 1N HCl. The resulting mixture was concentrated under vacuum, adjusted to pH=14 by addition of NaOH(aq), and extracted with 2×50 mL of CH₂Cl₂. The combined organic layers were dried over anhydrous sodium sulfate, and concentrated under vacuum, resulting in 0.2 g (65%) of product as crude yellow oil.

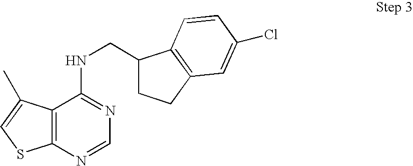

### N-((5-chloro-2,3-dihydro-1H-inden-1-yl)methyl)-5-methylthieno[2,3-d]pyrimidin-4-amine

A 100-mL round-bottom flask, under nitrogen, was charged with (5-chloro-2,3-dihydro-1H-inden-1-yl)methanamine (1.5 g, 5.55 mmol, 1.58 equiv, 67% pure), EtOH (50 mL), triethylamine (3 mL), and 4-bromo-5-methylthieno[2,3-d]pyrimidine (80 mg, 3.51 mmol). The resulting solution was stirred overnight at reflux. Reaction progress was monitored by TLC (ethyl acetate/petroleum ether=1:2). The resulting mixture was concentrated under vacuum, and purified by silica gel column chromatography, eluting with ethyl acetate/petroleum ether (1:10). This gave 36 mg (3%) product as a white solid. ¹H NMR (300 MHz, DMSO-d6) δ: 8.33 (1H, s), 7.27 (4H, m), 6.82 (1H, s), 3.81 (1H, s), 3.59 (2H, s), 2.87 (2H, m), 2.55 (3H, m), 2.20 (1H, m), 1.93 (1H, m). LCMS: 330 (M+1)⁺.

### EXAMPLE 31

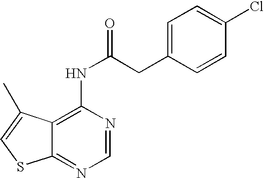

### 2-(4-chlorophenyl)-N-(5-methylthieno[2,3-d]pyrimidin-4-yl)acetamide

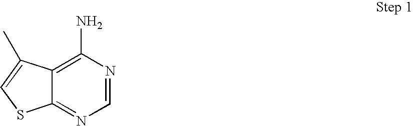

### 5-methylthieno[2,3-d]pyrimidin-4-amine

A 50-mL sealed tube was charged with 4-bromo-5-methylthieno[2,3-d]pyrimidine (500 mg, 2.19 mmol) and EtOH/NH₃(150 mL), then heated for 2 h at 100° C. Reaction progress was monitored by TLC (ethyl acetate/petroleum ether=1:2). The mixture was concentrated giving 0.58 g crude product as a pale yellow solid.

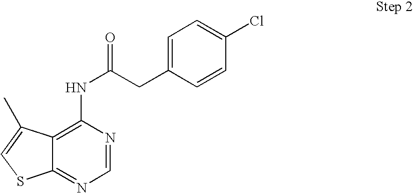

### 2-(4-chlorophenyl)-N-(5-methylthieno[2,3-d]pyrimidin-4-yl)acetamide

A 250-mL round-bottom flask was charged with 5-methylthieno[2,3-d]pyrimidin-4-amine (362 mg, 2.19 mmol), THF (100 ml, dry), NaH (351 mg, 14.62 mmol), followed by drop wise addition of a solution of 2-(4-chlorophenyl)acetyl chloride (1.65 g, 8.78 mmol) in THF (50 ml). The resulting solution was stirred for 4.5 hours at −5° C. Reaction progress was monitored by TLC (ethyl acetate/petroleum ether=1/1). The reaction was quenched by the addition of ice/salt. The pH was adjusted to 7-8 with NaHCO₃, then extracted with 3×150 mL of ethyl acetate. The combined organic layers were dried with magnesium sulfate, concentrated, and purified by column chromatography, eluting with ethyl acetate/petroleum ether (1/3). This gave 148 mg (21%) as a pale yellow solid. ¹H NMR (300 MHz, CDCl₃) δ: 2.27 (3H, s), 3.83 (2H, s), 7.43 (5H, t, J=9.6), 8.88 (1H, s), 10.76 (1H, s). LCMS: 318.1(M+1)⁺.

### EXAMPLE 32

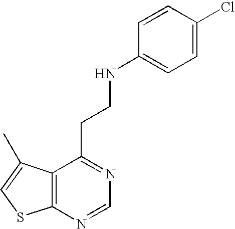

### 4-chloro-N-(2-(5-methylthieno[2,3-d]pyrimidin-4-yl)ethyl)aniline

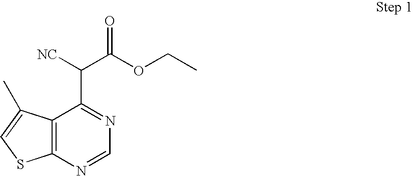

### ethyl 2-cyano-2-(5-methylthieno[2,3-d]pyrimidin-4-yl)acetate

A 50-mL round-bottom flask under nitrogen was was charged with 4-bromo-5-methylthieno[2,3-d]pyrimidine (1.14 g, 4.50 mmol), DMF (20 mL), ethyl 2-cyanoacetate (1.1 g, 9.73 mmol), CuI (95 mg, 0.50 mmol), Cs₂CO₃ (4.8 g, 14.72 mmol), and picolinic acid (120 mg, 0.98 mmol). The resulting solution was stirred overnight at 100° C. in an oil bath. Reaction progress was monitored by TLC (ethyl acetate/petroleum ether=1:3). The reaction mixture was cooled, quenched with 200 mL of water, extracted with 4×200 mL of ethyl acetate, dried over anhydrous sodium sulfate, concentrated under vacuum, and purified by column chromatography with ethyl acetate/petroleum ether (1:8). This gave 1 g (77%) of product as a yellow solid.

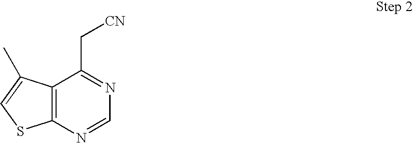

### 2-(5-methylthieno[2,3-d]pyrimidin-4-yl)acetonitrile

A 100-mL round-bottom flask was charged with ethyl 2-cyano-2-(5-methylthieno[2,3-d]pyrimidin-4-yl)acetate (2 g, 7.51 mmol), DMSO (20 mL), NaCl (2.834 g, 47.9 mmol), and H₂O (4 mL). The resulting mixture was stirred for 5 hours at 140° C. Reaction progress was monitored by TLC (ethyl acetate/petroleum ether=1:2). The reaction mixture was cooled to room temperature and diluted with 500 ml of H₂O/ice, then extracted with 2×60 ml of ethyl acetate. The combined organics were washed with 2×30 mL of saturated brine solution, dried over MgSO4, concentrated, and purified by column chromatography with ethyl acetate/petroleum ether (10:1). This gave 1 g (70%) of desired product as a white solid.

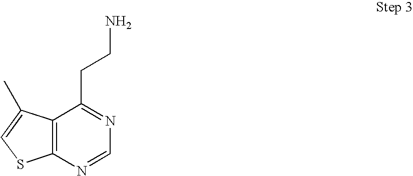

### 2-(5-methylthieno[2,3-d]pyrimidin-4-yl)ethanamine

A 100-mL round-bottom flask was vacuum flushed with a hydrogen gas, then charged with 2-(5-methylthieno[2,3-d]pyrimidin-4-yl)acetonitrile (480 mg, 2.29 mmol), methanol (30 mL), Pd/C (500 mg, 10%), and HCl (0.8 mL, 4.00 equiv, 30% aq). The mixture was stirred overnight at room temperature. Pd/C was removed by filtration. The filtrate was concentrated, giving 430 mg (97%) of the desired crude product as a brown solid. This material was used in the next step without further purification.

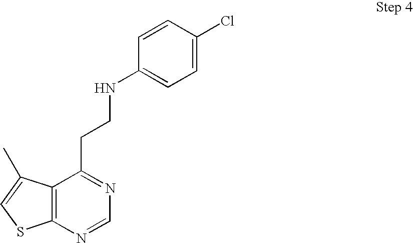

### 4-chloro-N-(2-(5-methylthieno[2,3-d]pyrimidin-4-yl)ethyl)aniline

A solution of 2-(5-methylthieno[2,3-d]pyrimidin-4-yl)ethanamine hydrochloride (100 mg, 0.33 mmol) in CH₂Cl₂ (100 mL), was treated with 4-chlorophenylboronic acid (136.3 mg, 0.87 mmol), Cu(OAc)₂ (78.6 mg, 0.44 mmol), Et₃N (88.2 mg, 0.87 mmol), and molecular sieves 4A (0.3 g). The resulting mixture was allowed stirred overnight at room temperature. Na₂S (2 g) was added and the reaction was stirred for an additional 1 hour at room temperature. Reaction progress was monitored by TLC (ethyl acetate/petroleum ether=1:1). The reaction was washed with 2×50 mL of H₂O, dried over Na₂SO₄, concentrate, and purified by column chromatography, eluting with ethyl acetate/petroleum ether (50:1). This gave 22 mg (22%) of the desired product as a yellow solid. ¹H NMR (300 MHz, CDCl₃) δ: 8.90 (1H, s), 7.37 (1H, s), 7.04 (2H, m), 6.61 (2H, m), 3.62 (2H, m), 3.32 (2H, m), 2.21 (3H, s). LCMS: 304.8 (M+1)⁺.

### EXAMPLE 33

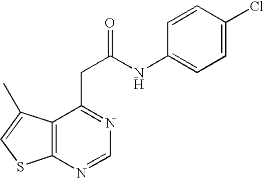

### N-(4-chlorophenyl)-2-(5-methylthieno[2,3-d]pyrimidin-4-yl)acetamide

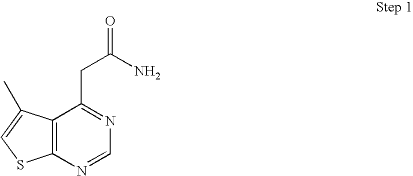

### 2-(5-methylthieno[2,3-d]pyrimidin-4-yl)acetamide

A 10-mL round-bottom flask was charged with 2-(5-methylthieno[2,3-d]pyrimidin-4-yl)acetonitrile (50 mg, 0.25 mmol, preparation previously described), ethanol (0.5 mL), H₂O (0.1 mL), and conc. HCl (0.25 mL), then stirred for 6 hours at 25° C. Reaction progress was monitored by TLC (ethyl acetate/petroleum ether=1:3). The reaction was quenched by adjusting the pH of the solution to 9 with NaHCO₃ (aq). This solution was extracted with 2×10 mL of CH₂Cl₂. The combined organics were dried over anhydrous sodium sulfate. The crude product was recrystallized from EA, resulting in 40 mg (73.0%) of 2product as a white solid.

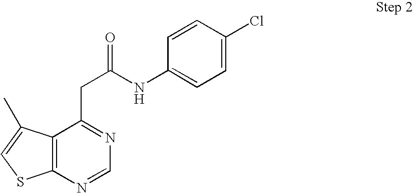

### N-(4-chlorophenyl)-2-(5-methylthieno[2,3-d]pyrimidin-4-yl)acetamide

A 5-mL sealed tube was purged with nitrogen, then charged with 2-(5-methylthieno[2,3-d]pyrimidin-4-yl)acetamide (100 mg, 0.47 mmol), DMF (2 mL), 1-chloro-4-iodobenzene (120 mg, 0.49 mmol), CuI (10 mg, 0.05 mmol), K₂CO₃ (140 mg, 1.00 mmol), and 2-(dimethylamino)acetic acid (12 mg, 0.08 mmol). The resulting solution was stirred for 15 hours at 70° C. Reaction progress was monitored by TLC (ethyl acetate/petroleum ether=1:3). The reaction was cooled to room temperature, diluted with 10 mL of H₂O, and extracted with 2×10 mL of ethyl acetate. The combined organic layers were dried over anhydrous sodium sulfate, concentrated, and purified by column chromatography (ethyl acetate/petroleum ether=1:3), resulting in 20 mg (13%) of product as a pale yellow solid. ¹H NMR (300 MHz, CDCl₃) δ: 2.75 (s, 3H), 4.33 (s, 2H), 7.24-7.28 (m, 3H), 7.49-7.52 (m, 2H), 9.04 (s, 1H), 9.55 (s, 1H). LCMS: 318 (M+1)³⁰ .

### EXAMPLE 34

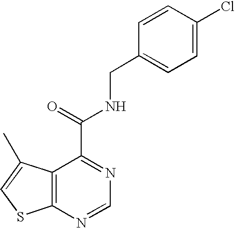

### N-(4-chlorobenzyl)-5-methylthieno[2,3-d]pyrimidine-4-carboxamide

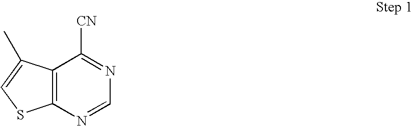

### 5-methylthieno[2,3-d]pyrimidine-4-carbonitrile

A 100-mL round-bottom flask under nitrogen was charged with 4-chloro-5-methylthieno[2,3-d]pyrimidine (1 g, 5.43 mmol), DMA (80 mL), Zn(CN)₂ (400 mg, 3.45 mmol), dppf(PdCl₂)CHCl₃ (50 mg, 0.06 mmol), Zn (42 mg, 0.65 mmol), and Pd₂(dba)₃ (50 mg, 0.05 mmol). The resulting solution was stirred for 2 hours at 150° C. Reaction progress was monitored by TLC (ethyl acetate/petroleum ether=1:3). The reaction was then quenched by adding of 150 mL of water/ice, then extracted with 3×200 mL of ethyl acetate, dried over anhydrous magnesium sulfate, concentrated under vacuum, and purified by silica gel column chromatography, eluted with ethyl acetate/petroleum ether (1:15). This gave 0.8 g (84%) of the product as a yellow solid.

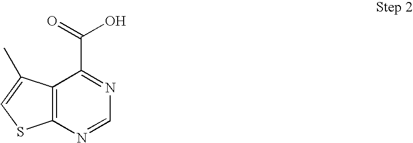

### 5-methylthieno[2,3-d]pyrimidine-4-carboxylic acid

A 100-mL round-bottom flask was charged with 5-methylthieno[2,3-d]pyrimidine-4-carbonitrile (500 mg, 2.86 mmol), H₂O (70 mL), and NaOH (140 mg, 3.50 mmol). The resulting solution was tirred overnight at reflux. Reaction progress was monitored by TLC (ethyl acetate/petroleum ether=1:1). The reaction was stopped by adjusting the pH to 1 by adding HCl (6M). Solids formed and were collected by filtration. This gave 0.4 g (72%) of product as a yellow solid.

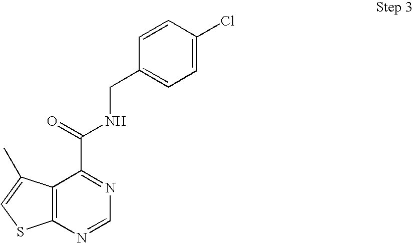

### N-(4-chlorobenzyl)-5-methylthieno[2,3-d]pyrimidine-4-carboxamide

A 250-mL round-bottom flask was charged with (4-chlorophenyl)methanamine (500 mg, 3.55 mmol, DMF (80 mL), triethylamine (460 mg, 4.55 mmol), and 5-methylthieno[2,3-d]pyrimidine-4-carboxylic acid (400 mg, 2.06 mmol). To resulting solution was added HATU (800 mg, 2.11 mmol). The reaction was stirred overnight at room temperature. The reaction progress was monitored by TLC (ethyl acetate/petroleum ether=1:1). The mixture was concentrated and purified by silica gel column chromatography, gradient eluted with ethyl acetate/petroleum ether (1:10 to 1:3). This resulted in 222 mg (34%) of product as a white solid. ¹H NMR (300 MHz, CDCl₃) δ: 9.44 (1H, s), 9.11 (1H, s), 7.71 (1H, s), 7.44 (4H, m), 4.55 (2H, d, J=6.3 Hz), 2.30 (3H, s). LCMS: 318 (M+1)⁺.

### EXAMPLE 35

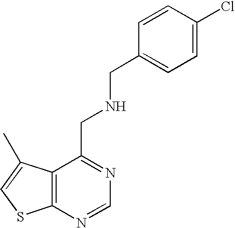

### N-(4-chlorobenzyl)-1-(5-methylthieno[2,3-d]pyrimidin-4-yl)methanamine

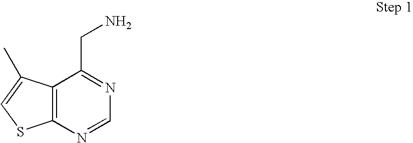

### (5-methylthieno[2,3-d]pyrimidin-4-yl)methanamine

A 100-mL 3-necked round-bottom flask under hydrogen atmosphere, was charged with 5-methylthieno[2,3-d]pyrimidine-4-carbonitrile (200 mg, 1.14 mmol), MeOH (50 mL), and Pd/C (0.1 g). To the mixture was added HCl (0.3 mL). The mixture was stirred overnight at room temperature. Reaction progress was monitored by TLC (CH₂Cl₂:MeOH=10:1). Catalyst was removed by filtration. The mother liquor was concentrated, dissolved in 30 ml of H₂O, pH adjusted to 10 with NH₄OH, extracted with 4×50 mL of dichloromethane. The organic layers were combined, dried over anhydrous sodium sulfate, concentrated under vacuum, giving 0.2 g (98%) of crude product as a purple solid.

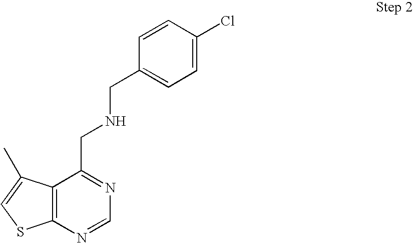

### N-(4-chlorobenzyl)-1-(5-methylthieno[2,3-d]pyrimidin-4-yl)methanamine

A 100-mL round-bottom flask was charged with (5-methylthieno[2,3-d]pyrimidin-4-yl)methanamine (300 mg, 1.68 mmol), EtOH (50 mL), and 4-chlorobenzaldehyde (270 mg, 1.93 mmol). The reaction was stirred overnight at reflux. The temperature was then dropped to <0° C. with an ice/salt bath, where NaBH₄ (100 mg, 2.63 mmol, 1.57 equiv) was added. This solution was stirred for an additional 1-hour at <0° C. Reaction progress was monitored by TLC (ethyl acetate/petroleum ether=1:1). The mixture was concentrated and purified by silica gel column chromatography, eluting with ethyl acetate/petroleum ether (1:10-1:2). This gave 6 mg (1%) of the product as a yellow solid. ¹H NMR (300 MHz, CDCl₃) δ: 8.97 (1H, s), 7.56 (1H, s), 7.37 (4H,s), 4.23 (2H, s), 3.80 (2H, s), 2.60 (3H, d, J=8.4). LCMS: 304 (M+1)⁺.

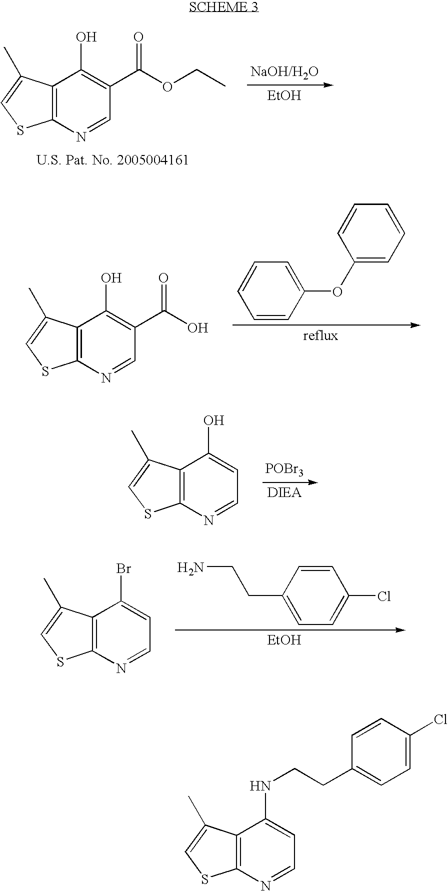

### EXAMPLE 36

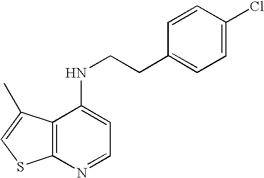

### N-(4-chlorophenethyl)-3-methylthieno[2,3-b]pyridin-4-amine

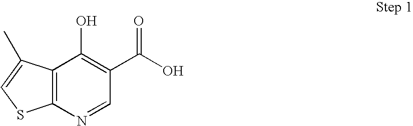

### 4-hydroxy-3-methylthieno[2,3-b]pyridine-5-carboxylic acid

A 250-mL round-bottom flask was charged with ethyl 4-hydroxy-3-methylthieno[2,3-b]pyridine-5-carboxylate (2 g, 8.44 mmol, prepared as described in patent US 2005004161), EtOH (100 mL), and sodium hydroxide (1.69 g, 42.25 mmol) dissolved in H₂O (100 mL). The resulting solution was stirred for 3.5 hours at 100° C. Reaction progress was monitored by TLC (CH₂Cl₂:MeOH=20:1). The reaction mixture was concentrated, mixed with ice, and pH adjusted to <1 with HCl. Solids formed and were collected by filtration. After drying, this gave 1.5 g (85%) of crude product as a white solid.

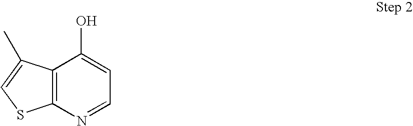

### 3-methylthieno[2,3-b]pyridin-4-ol

A 250-mL round-bottom flask was charged with 4-hydroxy-3-methylthieno[2,3-b]pyridine-5-carboxylic acid (1.5 g, 7.18 mmol) in 1-phenoxybenzene (20 mL). The resulting mixture was stirred and heated to for 15 minutes. Reaction progress was monitored by TLC (CH₂Cl₂:MeOH=10/1). The reaction was cooled to room temperature, and diluted with 20 mL of petroleum ether. Solids formed and were collected by filtration. The solid was dried giving 1.1 g (93%) of product as a light yellow powder.

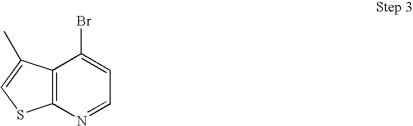

### 4-bromo-3-methylthieno[2,3-b]pyridine

A 250-mL 3-necked round bottom flask was charged with 3-methylthieno[2,3-b]pyridin-4-ol (1.1 g, 6.67 mmol), diisopropylethylamine (200 mL), and phosphoryl tribromide (5.68 g, 20.00 mmol, 3.00 equiv). The resulting solution was stirred for 2 hours at 100° C. The reaction mixture was cooled, concentrated and purified by silica gel chromatography with ethyl acetate/petroleum ether (1/10). This gave 1.3 g (86%) of product as a light yellow solid.

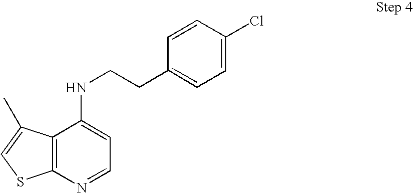

### N-(4-chlorophenethyl)-3-methylthieno[2,3-b]pyridin-4-amine

A 5-mL sealed tube was charged with 4-bromo-3-methylthieno[2,3-b]pyridine (300 mg, 1.32 mmol), EtOH (1 ml), and 2-(4-chlorophenyl)ethanamine (2 mL). The resulting solution was stirred for 7 hours at 145° C., and then 17 hours at 120° C. in an oil bath. Reaction progress was monitored by TLC (ethyl acetate/petroleum ether=1/2). The reaction was concentrated under vacuum, and purified by column silica gel eluted with ethyl acetate/petroleum ether (1:10). This gave 102 mg (26%) of N-(4-chlorophenethyl)-3-methylthieno[2,3-b]pyridin-4-amine as a light yellow solid. ¹H NMR (300 MHz, CDCl₃) δ: 2.51 (3H, s), 2.95 (2H, t, J=7.5 Hz), 3.48 (2H, dd, J=6.6 Hz), 5.91 (1H, t, J=5.4 Hz), 6.54 (1H, d, J=5.7 Hz), 7.04 (1H, s), 7.32-7.40 (4H, m), 8.07 (1H, d, J=5.4 Hz). LCMS: 302 (M+1)⁺.

### EXAMPLE 37

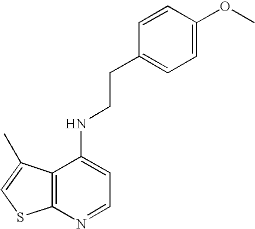

### N-(4-methoxyphenethyl)-3-methylthieno[2,3-b]pyridin-4-amine

The title compound was prepared analogously to EXAMPLE 36, where 2-(4-methoxyphenyl)ethanamine was substituted for 2-(4-chlorophenyl) ethanamine in the final step of that sequence. ¹H NMR (300 MHz, CDCl₃) δ: 2.51 (3H, s), 2.89 (2H, t, J=7.2 Hz), 3.44 (2H, dd, J=12.6, 6.6 Hz), 3.74 (1H, s), 5.85 (1H, t, J=5.4 Hz), 6.52 (1H, d, J=5.7 Hz), 6.89 (2H, d, J=8.7 Hz), 7.04 (1H, s), 7.32 (2H, d, J=8.4 Hz), 8.08 (1H, d, J=5.4 Hz). LCMS: 298 (M+1)⁺.

### EXAMPLE 38

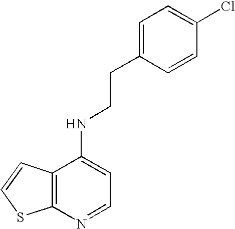

### N-(4-chlorophenethyl)thieno[2,3-b]pyridin-4-amine

The title compound was prepared analogously to EXAMPLE 36, where 4-hydroxy-3-methylthieno[2,3-b]pyridine-5-carboxylic acid was substituted for 4-hydroxythieno[2,3-b]pyridine-5-carboxylic acid in the first step of the sequence. ¹H NMR (300 MHz, CDCl₃) δ: 2.88 (2H, t, J=7.2 Hz), 3.34 (2H, dd, J=12.6, 6.6 Hz), 3.74 (1H, s), 5.85 (1H, t, J=5.4 Hz), 6.52 (1H, d, J=5.7 Hz), 6.77 (d, J=1.2 Hz, 1H), 6.89 (2H, d, J=8.7 Hz), 7.04 (d, J=1.4 Hz, 1H), 7.32 (2H, d, J=8.4 Hz), 8.08 (1H, d, J=5.4 Hz). LCMS: 288 (M+1)⁺.

### EXAMPLE 39

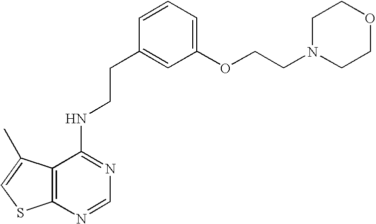

### 5-methyl-N-(3-(2-morpholinoethoxy)phenethyl)thieno[2,3-d]pyrimidin-4-amine

The title compound was prepared analogously to EXAMPLE 16, where 2-(3-(2-morpholinoethoxy)phenyl)ethanamine was substituted for 2-(3-methoxyphenyl)ethylamine. LCMS: 399.2 (M+1)⁺.

### EXAMPLE 40

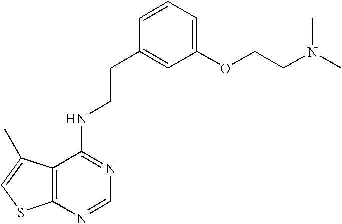

### N-(3-(2-(dimethylamino)ethoxy)phenethyl)-5-methylthieno[2,3-d]pyrimidin-4-amine

The title compound was prepared analogously to EXAMPLE 16, where 23-methylcyclohexanamine was substituted for 2-(3-methoxyphenyl)ethylamine. LCMS: 357.1 (M+1)⁺.

### EXAMPLE 41

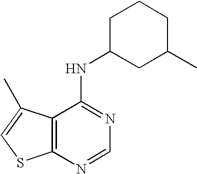

### 5-methyl-N-(3-methylcyclohexyl)thieno[2,3-d]pyrimidin-4-amine

The title compound was prepared analogously to EXAMPLE 16, where 23-methylcyclohexanamine was substituted for 2-(3-methoxyphenyl)ethylamine. LCMS: 262.1 (M+1)⁺.

### Compounds Prepared by Parallel Synthesis

The invention is illustrated by the following Scheme:

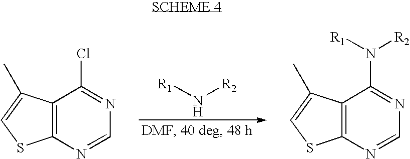

Primary and secondary amine monomers (4 μmol) in DMF (8 μL) were transferred to each well of a 384 well plate, then treated with a solution of 4-chloro-5-methylthieno[2,3-d]pyrimidine (4.0 μmol) in DMF (10 μL). The reaction plate was then heat sealed, shaken, and placed in a 40 deg water bath for 48 hours. Solvent was removed using a centrifugal evaporator. Select products were analyzed for purity by LCMS before testing.

TABLE 1
Primary Amine Monomers

| | | |
|---|---|---|
| methylamine | N-(2- | 4-(3- |
| | aminoethyl)pyrrolidine | aminopropyl)morpholine |
| ethylamine | 1-amino-4-methylpiperazine | N,N- |
| | | diisopropylethylenediamine |
| prop-2-ynylamine | N,N-diethylethylenediamine | 1,2,3,4-tetrahydro-1- |
| | | naphthylamine |
| aminoacetonitrile | 3-methylbenzylamine | 1-methyl-3- |
| | | phenylpropylamine |
| cyclopropylamine | (S)-(−)-alpha- | 4-isopropylbenzylamine |
| | methylbenzylamine | |
| isopropylamine | (R)-(+)-alpha- | piperonylamine |
| | methylbenzylamine | |
| propylamine | phenethylamine | 3- |
| | | methoxyphenethylamine |
| ethylenediamine | 2-methylbenzylamine | L(−)-2-amino-3-phenyl- |
| | | 1-propanol |
| ethanolamine | 4-methylbenzylamine | 4- |
| | | methoxyphenethylamine |
| 3-aminopropionitrile | 2-(aminomethyl)-5- | 2- |
| | methylpyrazine | methoxyphenethylamine |
| cyclobutylamine | 3-fluorobenzylamine | 2-ethoxybenzylamine |
| aminomethylcyclopropane | 4-fluorobenzylamine | 4-aminoveratrole |
| sec-butylamine | 2-fluorobenzylamine | 2-(2- |
| | | chlorophenyl)ethylamine |
| isobutylamine | 1-(3-aminopropyl)imidazole | 2-(4- |
| | | chlorophenyl)ethylamine |
| 3-amino-1-propanol | 2-(2-aminoethyl)-1- | 2-(3- |
| | methylpyrrolidine | chlorophenyl)ethylamine |
| DL-2-amino-1-propanol | 1-(2-aminoethyl)-piperidine | 4-amino-2,2,6,6- |
| | | tetramethylpiperidine |
| 2-methoxyethylamine | 1,5-dimethylhexylamine | 2-amino-5- |
| | | diethylaminopentane |
| cyclopentylamine | 4-(2-aminoethyl)morpholine | 3-chloro-4- |
| | | fluorobenzylamine |
| 2-amino pentane | ethyl-3-aminobutyrate | 3-(benzylamino)- |
| | | propionitrile |
| 2-methylbutylamine | 1-aminoindan | N-(2- |
| | | aminoethyl)carbamic |
| | | acid tert-butyl ester |
| 1,2-dimethylpropylamine | 2-(p-tolyl)ethylamine | tryptamine |
| isoamylamine | 2,5-dimethylbenzylamine | 4-tert-butylbenzylamine |
| N,N- | (S)-(−)-alpha,4- | N-(3-aminopropyl)-n- |
| dimethylethylenediamine | dimethylbenzylamine | methylaniline |
| 2-amino-1-methoxy- | (r)-(+)-alpha,4- | 1-aminomethyl-1- |
| propane | dimethylbenzylamine | cyclohexanol |
| 2-amino-1-butanol | beta-methylphenethylamine | 3,5- |
| | | dimethoxybenzylamine |
| DL-2-amino-1-butanol | 3-phenyl-1-propylamine | 3,4- |
| | | dimethoxybenzylamine |
| 4-amino-1-butanol | 3,4-dimethylbenzylamine | 2-aminoacetophenone |
| furfurylamine | 3-methylphenethylamine | ethyl 4-amino-1- |
| | | piperidinecarboxylate |
| cyclohexylamine | 4-(ethylaminomethyl)- | N-(3- |
| | pyridine | aminopropyl)carbamic |
| | | acid tert-butyl ester |
| tetrahydrofurfurylamine | tyramine | 4- |
| | | (trifluoromethyl)benzylamine |
| 3,3-dimethylbutylamine | 2-phenoxyethylamine | 3- |
| | | (trifluoromethyl)benzylamine |
| 1,3-dimethylbutylamine | 2-methoxybenzylamine | 3,4-dichlorobenzylamine |
| D-cycloserine | 4-methoxybenzylamine | 2,4-dichlorobenzylamine |
| 4-aminomorpholine | 3-methoxybenzylamine | 1-benzyl-3- |
| | | aminopyrrolidine |
| DL-2-amino-3-methyl-1- | 4-fluorophenethylamine | 2,5- |
| butanol | | dimethoxyphenethylamine |
| benzylamine | 2-fluorophenethylamine | 4- |
| | | dimethylaminobenzylamine |
| 3-(aminomethyl)pyridine | 3-fluorophenethylamine | 3-hydroxy-4- |
| | | methoxybenzylamine |
| 2-(aminomethyl)pyridine | 3-chlorobenzylamine | 4-amino-1- |
| | | benzylpiperidine |
| 4-(aminomethyl)pyridine | 2-chlorobenzylamine | 4′-fluoro-2′- |
| | | (trifluoromethyl)acetophenone |
| 2-thiophenemethylamine | 4-chlorobenzylamine | 4-(2- |
| | | aminoethyl)benzene- |
| | | sulfonamide |
| 4- | N-(3′-aminopropyl)-2- | 2-amino-4′- |
| methylcyclohexylamine | pyrrolidinone | methoxyacetophenone |
| cyclohexanemethylamine | 2,4-difluorobenzylamine | 3,4-methylenedioxy- |
| | | phenethylamine |
| cycloheptylamine | 3,4-difluorobenzylamine | 3,3-diphenylpropylamine |
| 2- | 3,5-difluorobenzylamine | N-(2-aminoethyl)-p- |
| methylcyclohexylamine | | toluenesulfonamide |

TABLE 2
Secondary Amine Monomers

| | | |
|---|---|---|
| dimethylamine | N-ethylbenzylamine | 1-(3,4- |
| | | dimethylphenyl)piperazine |
| N-ethylmethylamine | N- | 1-(2,4- |
| | methylphenethylamine | dimethylphenyl)piperazine |
| N-methylpropargylamine | N-ethylbenzylamine | 1-(2,5- |
| | | dimethylphenyl)piperazine |
| diethylamine | 2-(2- | 1-(2,3- |
| | methylaminoethyl)pyridine | dimethylphenyl)piperazine |
| N-methylpropylamine | 2-(2- | 1-(4- |
| | methylaminoethyl)pyridine | methoxyphenyl)piperazine |
| 2-(methylamino)ethanol | decahydroquinoline | 1-(2- |
| | | methoxyphenyl)piperazine |
| N-methyl-beta- | allylcyclohexylamine | 1-(3- |
| alaninenitrile | | methoxyphenyl)piperazine |
| morpholine | methyl isonipecotate | n- |
| | | methylhomoveratrylamine |
| N-methylisobutylamine | N-isopropylbenzylamine | 1-(3- |
| | | chlorophenyl)piperazine |
| N-ethylisopropylamine | N-benzylethanolamine | 1-(4- |
| | | chlorophenyl)piperazine |
| N-methylbutylamine | DL-alpha- | 1-(2,4-difluorophenyl)- |
| | (methylaminomethyl)benzyl | piperazine |
| | alcohol | |
| 2-(ethylamino)ethanol | 4-piperidone | 3,3′-dipicolylamine |
| | monohydrate | |
| | hydrochloride | |
| thiazolidine | ethyl isonipecotate | 1-[2-(morpholin-4- |
| | | yl)ethyl]piperazine |
| 2-methylpiperidine | ethyl nipecotate | 3-(N-tert-butoxycarbonyl- |
| | | N- |
| | | methylamino)pyrrolidine |
| 4-methylpiperidine | ethyl 1- | 4′-piperazinoacetophenone |
| | piperazinecarboxylate | |
| 1-methylpiperazine | 3- | N′-benzyl-N,N- |
| | (benzylamino)propionitrile | diethylethylenediamine |
| L-prolinol | 3-(3 | 1-(4- |
| | pyridylmethylamino)propionitrile | nitrophenyl)piperazine |
| H-D-Pro-ol | 1-phenylpiperazine | N-methyl-1- |
| | | naphthalenemethylamine |
| | | hydrochloride |
| N-ethyl-N-butylamine | 1-(2-pyridyl)piperazine | 1-(5-chloro-2- |
| | | methylphenyl)-piperazine |
| dipropylamine | N-butylbenzylamine | N-methyl-3,4,5- |
| | | trimethoxybenzylamine |
| thiomorpholine | (S)-(−)-N-(2- | N-benzyl-2- |
| | hydroxyethyl)-alpha- | phenethylamine |
| | phenylethylamine | |
| diethanolamine | 3-(2- | 1,2-diphenylethyl-N- |
| | pyridylmethylamino)-1- | methylamine |
| | propanol | |
| 3,5-dimethylpiperidine | synephrine | N-benzyl-2- |
| | | phenylethylamine |
| N-methylcyclohexylamine | 1-cyclohexylpiperazine | 4-(4-chlorophenyl)-4- |
| | | hydroxypiperidine |
| cis-2,6-dimethylpiperidine | 4-benzylpiperidine | N-(2,4- |
| | | dichlorobenzyl)propargylamine |
| 3- | 1-benzylpiperazine | N-(alpha-benzylfurfuryl)- |
| (dimethylamino)pyrrolidine | | ethylamine |
| 2,6-dimethylpiperidine | 1-benzyl-hexahydro- | (S)-4-n-hydroxymethyl 1- |
| | pyrimidine | piperazine |
| 3-piperidinemethanol | 1-(4-hydroxyphenyl)- | 1-(4-tert-butyl-phenyl)- |
| | piperazine | piperazine |
| | dihydrobromide | |
| (S)-(+)- | n′-benzyl-N,N- | 1-piperonylpiperazine |
| (methoxymethyl)pyrrolidine | dimethylethylenediamine | |
| 2,6-dimethylmorpholine | 1-(2- | 6,7-dimethoxy-1,2,3,4- |
| | fluorophenyl)piperazine | tetrahydro-isoquinoline |
| | | hydrochloride |
| 2-piperidinemethanol | 1-(4- | 4-piperazine- |
| | fluorophenyl)piperazine | trifluoromethyl benzene |
| 4-ethylamino-1-butanol | 1- | N-(alpha,alpha,alpha- |
| | (cyclohexylmethyl)piperazine | trifluoro-m- |
| | | tolyl)piperazine |
| isoindoline | L(−)-epinephrine | 1-(3,4- |
| | | dichlorophenyl)piperazine |
| N-methylbenzylamine | ethyl 2-(3-oxo- | 1-(3,4- |
| | 2piperazinyl)acetate | dichlorophenyl)piperazine |
| N-ethylcyclohexylamine | 1-boc-piperazine | 1-[5- |
| | | (trifluoromethyl)pyrid-2- |
| | | yl]piperazine |
| isonipecotamide | 3-(tert- | 1-(2- |
| | butoxycarbonylamino)pyrrolidine | chlorophenyl)piperazine |
| | | hydrochloride |
| (3S)-(−)-3- | 1-(2-cyanophenyl)- | (S)-1-(3,4-1-(3,4- |
| acetamidopyrrolidine | piperazine | dimethoxyphenyl)piperazine |
| | | hydrochloride |
| nipecotamide | 6- | 3-(4- |
| | piperazinonicotinonitrile | trifluoromethylphenyl)piperidine |
| | | hydrochloride |
| (3R)-(+)-3- | 1-(2,3- | 1-(4- |
| acetamidopyrrolidine | dimethylphenyl)piperazine | chlorophenyl)piperazine |
| | | dihydrochloride |
| 2-piperidineethanol | 2-methyl-1-(3- | |
| | methylphenyl)piperazine | |
| 1,2,3,4- | 1-(2- | |
| tetrahydroisoquinoline | phenylethyl)piperazine | |

TABLE 3
EXAMPLES 42 through 288

| Example | | |
|---|---|---|
| Number | Smiles | Name |
| EXAMPLE 42 | Fc1ccccc1CCNc2ncnc3sc4CCCCc4c23 | N-[2-(2-fluorophenyl)ethyl]-8-thia-4,6- |
| | | diazatricyclo[7.4.0.0\hat{ }{2,7}]trideca- |
| | | 1(9),2(7),3,5-tetraen-3-amine |
| EXAMPLE 43 | Cc1sc2ncnc(NCCc3ccccc3Cl)c2c1C | N-[2-(2-chlorophenyl)ethyl]-5,6- |
| | | dimethylthieno[2,3-d]pyrimidin-4-amine |
| EXAMPLE 44 | CCOC(═O)C1CCN(CC1)c2ncnc3scc(C)c23 | ethyl 1-{5-methylthieno[2,3-d]pyrimidin-4- |
| | | yl}piperidine-4-carboxylate |
| EXAMPLE 45 | Clc1ccc(CCNc2ncnc3sccc23)cc1 | N-[2-(4-chlorophenyl)ethyl]thieno[2,3- |
| | | d]pyrimidin-4-amine |
| EXAMPLE 46 | Cc1csc2ncnc(NCCc3ccc(Cl)cc3)c12 | N-[2-(4-chlorophenyl)ethyl]-5- |
| | | methylthieno[2,3-d]pyrimidin-4-amine |
| EXAMPLE 47 | CCCCNc1ncnc2scc(−c3ccccc3)c12 | N-butyl-5-phenylthieno[2,3-d]pyrimidin-4- |
| | | amine |
| EXAMPLE 48 | COc1ccc(CCNc2ncnc3sccc23)cc1 | N-[2-(4-methoxyphenyl)ethyl]thieno[2,3- |
| | | d]pyrimidin-4-amine |
| EXAMPLE 49 | CNc1ncnc2scc(C)c12 | N,5-dimethylthieno[2,3-d]pyrimidin-4- |
| | | amine |
| EXAMPLE 50 | CCNc1ncnc2scc(C)c12 | N-ethyl-5-methylthieno[2,3-d]pyrimidin-4- |
| | | amine |
| EXAMPLE 51 | Cc1csc2ncnc(NCC#C)c12 | 5-methyl-N-(prop-2-yn-1-yl)thieno[2,3- |
| | | d]pyrimidin-4-amine |
| EXAMPLE 52 | Cc1csc2ncnc(NCC#N)c12 | 2-({5-methylthieno[2,3-d]pyrimidin-4- |
| | | yl}amino)acetonitrile |
| EXAMPLE 53 | Cc1csc2ncnc(NC3CC3)c12 | N-cyclopropyl-5-methylthieno[2,3- |
| | | d]pyrimidin-4-amine |
| EXAMPLE 54 | CCCNc1ncnc2scc(C)c12 | 5-methyl-N-propylthieno[2,3-d]pyrimidin- |
| | | 4-amine |
| EXAMPLE 55 | Cc1csc2ncnc(NCCN)c12 | N-(2-aminoethyl)-5-methylthieno[2,3- |
| | | d]pyrimidin-4-amine |
| EXAMPLE 56 | Cc1csc2ncnc(NCCO)c12 | 2-({5-methylthieno[2,3-d]pyrimidin-4- |
| | | yl}amino)ethan-1-ol |
| EXAMPLE 57 | Cc1csc2ncnc(NCCC#N)c12 | 3-({5-methylthieno[2,3-d]pyrimidin-4- |
| | | yl}amino)propanenitrile |
| EXAMPLE 58 | Cc1csc2ncnc(NC3CCC3)c12 | N-cyclobutyl-5-methylthieno[2,3- |
| | | d]pyrimidin-4-amine |
| EXAMPLE 59 | Cc1csc2ncnc(NCC3CC3)c12 | N-(cyclopropylmethyl)-5-methylthieno[2,3- |
| | | d]pyrimidin-4-amine |
| EXAMPLE 60 | CCC(C)Nc1ncnc2scc(C)c12 | N-(butan-2-yl)-5-methylthieno[2,3- |
| | | d]pyrimidin-4-amine |
| EXAMPLE 61 | CC(C)CNc1ncnc2scc(C)c12 | 5-methyl-N-(2-methylpropyl)thieno[2,3- |
| | | d]pyrimidin-4-amine |
| EXAMPLE 62 | Cc1csc2ncnc(NCCCO)c12 | 3-({5-methylthieno[2,3-d]pyrimidin-4- |
| | | yl}amino)propan-1-ol |
| EXAMPLE 63 | CC(CO)Nc1ncnc2scc(C)c12 | 2-({5-methylthieno[2,3-d]pyrimidin-4- |
| | | yl}amino)propan-1-ol |
| EXAMPLE 64 | COCCNc1ncnc2scc(C)c12 | N-(2-methoxyethyl)-5-methylthieno[2,3- |
| | | d]pyrimidin-4-amine |
| EXAMPLE 65 | Cc1csc2ncnc(NC3CCCC3)c12 | N-cyclopentyl-5-methylthieno[2,3- |
| | | d]pyrimidin-4-amine |
| EXAMPLE 66 | CCCC(C)Nc1ncnc2scc(C)c12 | 5-methyl-N-(pentan-2-yl)thieno[2,3- |
| | | d]pyrimidin-4-amine |
| EXAMPLE 67 | CCC(C)CNc1ncnc2scc(C)c12 | 5-methyl-N-(2-methylbutyl)thieno[2,3- |
| | | d]pyrimidin-4-amine |
| EXAMPLE 68 | CC(C)C(C)Nc1ncnc2scc(C)c12 | 5-methyl-N-(3-methylbutan-2- |
| | | yl)thieno[2,3-d]pyrimidin-4-amine |
| EXAMPLE 69 | CC(C)CCNc1ncnc2scc(C)c12 | 5-methyl-N-(3-methylbutyl)thieno[2,3- |
| | | d]pyrimidin-4-amine |
| EXAMPLE 70 | CN(C)CCNc1ncnc2scc(C)c12 | dimethyl[2-({5-methylthieno[2,3- |
| | | d]pyrimidin-4-yl}amino)ethyl]amine |
| EXAMPLE 71 | COCC(C)Nc1ncnc2scc(C)c12 | N-(1-methoxypropan-2-yl)-5- |
| | | methylthieno[2,3-d]pyrimidin-4-amine |
| EXAMPLE 72 | CCC(CO)Nc1ncnc2scc(C)c12 | 2-({5-methylthieno[2,3-d]pyrimidin-4- |
| | | yl}amino)butan-1-ol |
| EXAMPLE 73 | CCC(CO)Nc1ncnc2scc(C)c12 | 2-({5-methylthieno[2,3-d]pyrimidin-4- |
| | | yl}amino)butan-1-ol |
| EXAMPLE 74 | Cc1csc2ncnc(NCCCCO)c12 | 4-({5-methylthieno[2,3-d]pyrimidin-4- |
| | | yl}amino)butan-1-ol |
| EXAMPLE 75 | Cc1csc2ncnc(NCc3ccco3)c12 | N-(furan-2-ylmethyl)-5-methylthieno[2,3- |
| | | d]pyrimidin-4-amine |
| EXAMPLE 76 | Cc1csc2ncnc(NC3CCCCC3)c12 | N-cyclohexyl-5-methylthieno[2,3- |
| | | d]pyrimidin-4-amine |
| EXAMPLE 77 | Cc1csc2ncnc(NCC3CCCO3)c12 | 5-methyl-N-(oxolan-2-ylmethyl)thieno[2,3- |
| | | d]pyrimidin-4-amine |
| EXAMPLE 78 | Cc1csc2ncnc(NCCC(C)(C)C)c12 | N-(3,3-dimethylbutyl)-5-methylthieno[2,3- |
| | | d]pyrimidin-4-amine |
| EXAMPLE 79 | CC(C)CC(C)Nc1ncnc2scc(C)c12 | 5-methyl-N-(4-methylpentan-2- |
| | | yl)thieno[2,3-d]pyrimidin-4-amine |
| EXAMPLE 80 | Cc1csc2ncnc(NC3CONC3═O)c12 | 4-({5-methylthieno[2,3-d]pyrimidin-4- |
| | | yl}amino)-1,2-oxazolidin-3-one |
| EXAMPLE 81 | Cc1csc2ncnc(NN3CCOCC3)c12 | N-{5-methylthieno[2,3-d]pyrimidin-4- |
| | | yl}morpholin-4-amine |
| EXAMPLE 82 | CC(C)C(CO)Nc1ncnc2scc(C)c12 | 3-methyl-2-({5-methylthieno[2,3- |
| | | d]pyrimidin-4-yl}amino)butan-1-ol |
| EXAMPLE 83 | Cc1csc2ncnc(NCc3ccccc3)c12 | N-benzyl-5-methylthieno[2,3-d]pyrimidin- |
| | | 4-amine |
| EXAMPLE 84 | Cc1csc2ncnc(NCc3cccnc3)c12 | 5-methyl-N-(pyridin-3- |
| | | ylmethyl)thieno[2,3-d]pyrimidin-4-amine |
| EXAMPLE 85 | Cc1csc2ncnc(NCc3ccccn3)c12 | 5-methyl-N-(pyridin-2- |
| | | ylmethyl)thieno[2,3-d]pyrimidin-4-amine |
| EXAMPLE 86 | Cc1csc2ncnc(NCc3ccncc3)c12 | 5-methyl-N-(pyridin-4- |
| | | ylmethyl)thieno[2,3-d]pyrimidin-4-amine |
| EXAMPLE 87 | Cc1csc2ncnc(NCc3cccs3)c12 | 5-methyl-N-(thiophen-2- |
| | | ylmethyl)thieno[2,3-d]pyrimidin-4-amine |
| EXAMPLE 88 | | This example has intentionally been left |
| | | blank |
| EXAMPLE 89 | Cc1csc2ncnc(NCC3CCCCC3)c12 | N-(cyclohexylmethyl)-5-methylthieno[2,3- |
| | | d]pyrimidin-4-amine |
| EXAMPLE 90 | Cc1csc2ncnc(NC3CCCCCC3)c12 | N-cycloheptyl-5-methylthieno[2,3- |
| | | d]pyrimidin-4-amine |
| EXAMPLE 91 | CC1CCCCC1Nc2ncnc3scc(C)c23 | 5-methyl-N-(2- |
| | | methylcyclohexyl)thieno[2,3-d]pyrimidin- |
| | | 4-amine |
| EXAMPLE 92 | Cc1csc2ncnc(NCCN3CCCC3)c12 | 5-methyl-N-[2-(pyrrolidin-1- |
| | | yl)ethyl]thieno[2,3-d]pyrimidin-4-amine |
| EXAMPLE 93 | CN1CCN(CC1)Nc2ncnc3scc(C)c23 | 4-methyl-N-{5-methylthieno[2,3- |
| | | d]pyrimidin-4-yl}piperazin-1-amine |
| EXAMPLE 94 | CCN(CC)CCNc1ncnc2scc(C)c12 | diethyl[2-({5-methylthieno[2,3- |
| | | d]pyrimidin-4-yl}amino)ethyl]amine |
| EXAMPLE 95 | Cc1cccc(CNc2ncnc3scc(C)c23)c1 | 5-methyl-N-[(3- |
| | | methylphenyl)methyl]thieno[2,3- |
| | | d]pyrimidin-4-amine |
| EXAMPLE 96 | C[C@H](NC1ncnc2scc(C)c12)c3ccccc3 | 5-methyl-N-[(1S)-1- |
| | | phenylethyl]thieno[2,3-d]pyrimidin-4- |
| | | amine |
| EXAMPLE 97 | C[C@@H](Nc1ncnc2scc(C)c12)c3ccccc3 | 5-methyl-N-[(1R)-1- |
| | | phenylethyl]thieno[2,3-d]pyrimidin-4- |
| | | amine |
| EXAMPLE 98 | Cc1csc2ncnc(NCCc3ccccc3)c12 | 5-methyl-N-(2-phenylethyl)thieno[2,3- |
| | | d]pyrimidin-4-amine |
| EXAMPLE 99 | Cc1ccccc1CNc2ncnc3scc(C)c23 | 5-methyl-N-[(2- |
| | | methylphenyl)methyl]thieno[2,3- |
| | | d]pyrimidin-4-amine |
| EXAMPLE 100 | Cc1ccc(CNc2ncnc3scc(C)c23)cc1 | 5-methyl-N-[(4- |
| | | methylphenyl)methyl]thieno[2,3- |
| | | d]pyrimidin-4-amine |
| EXAMPLE 101 | Cc1cnc(CNc2ncnc3scc(C)c23)cn1 | 5-methyl-N-[(5-methylpyrazin-2- |
| | | yl)methyl]thieno[2,3-d]pyrimidin-4-amine |
| EXAMPLE 102 | Cc1csc2ncnc(NCc3cccc(F)c3)c12 | N-[(3-fluorophenyl)methyl]-5- |
| | | methylthieno[2,3-d]pyrimidin-4-amine |
| EXAMPLE 103 | Cc1csc2ncnc(NCc3ccc(F)cc3)c12 | N-[(4-fluorophenyl)methyl]-5- |
| | | methylthieno[2,3-d]pyrimidin-4-amine |
| EXAMPLE 104 | Cc1csc2ncnc(NCc3ccccc3F)c12 | N-[(2-fluorophenyl)methyl]-5- |
| | | methylthieno[2,3-d]pyrimidin-4-amine |
| EXAMPLE 105 | Cc1csc2ncnc(NCCCn3ccnc3)c12 | N-[3-(1H-imidazol-1-yl)propyl]-5- |
| | | methylthieno[2,3-d]pyrimidin-4-amine |
| EXAMPLE 106 | CN1CCCC1CCNc2ncnc3scc(C)c23 | 5-methyl-N-[2-(1-methylpyrrolidin-2- |
| | | yl)ethyl]thieno[2,3-d]pyrimidin-4-amine |
| EXAMPLE 107 | Cc1csc2ncnc(NCCN3CCCCC3)c12 | 5-methyl-N-[2-(piperidin-1- |
| | | yl)ethyl]thieno[2,3-d]pyrimidin-4-amine |
| EXAMPLE 108 | CC(C)CCCC(C)Nc1ncnc2scc(C)c12 | 5-methyl-N-(6-methylheptan-2- |
| | | yl)thieno[2,3-d]pyrimidin-4-amine |
| EXAMPLE 109 | Cc1csc2ncnc(NCCN3CCOCC3)c12 | 5-methyl-N-[2-(morpholin-4- |
| | | yl)ethyl]thieno[2,3-d]pyrimidin-4-amine |
| EXAMPLE 110 | CCOC(═O)CC(C)Nc1ncnc2scc(C)c12 | ethyl 3-({5-methylthieno[2,3-d]pyrimidin- |
| | | 4-yl}amino)butanoate |
| EXAMPLE 111 | Cc1csc2ncnc(NC3CCc4ccccc34)c12 | N-(2,3-dihydro-1H-inden-1-yl)-5- |
| | | methylthieno[2,3-d]pyrimidin-4-amine |
| EXAMPLE 112 | | This example has intentionally been left |
| | | blank |
| EXAMPLE 113 | Cc1ccc(C)c(CNc2ncnc3scc(C)c23)c1 | N-[(2,5-dimethylphenyl)methyl]-5- |
| | | methylthieno[2,3-d]pyrimidin-4-amine |
| EXAMPLE 114 | C[C@H](NC1ncnc2scc(C)c12)c3ccc(C)cc3 | 5-methyl-N-[(1S)-1-(4- |
| | | methylphenyl)ethyl]thieno[2,3- |
| | | d]pyrimidin-4-amine |
| EXAMPLE 115 | C[C@@H](Nc1ncnc2scc(C)c12)c3ccc(C)cc3 | 5-methyl-N-[(1R)-1-(4- |
| | | methylphenyl)ethyl]thieno[2,3- |
| | | d]pyrimidin-4-amine |
| EXAMPLE 116 | CC(CNc1ncnc2scc(C)c12)c3ccccc3 | 5-methyl-N-(2-phenylpropyl)thieno[2,3- |
| | | d]pyrimidin-4-amine |
| EXAMPLE 117 | Cc1csc2ncnc(NCCCc3ccccc3)c12 | 5-methyl-N-(3-phenylpropyl)thieno[2,3- |
| | | d]pyrimidin-4-amine |
| EXAMPLE 118 | Cc1ccc(CNc2ncnc3scc(C)c23)cc1C | N-[(3,4-dimethylphenyl)methyl]-5- |
| | | methylthieno[2,3-d]pyrimidin-4-amine |
| EXAMPLE 119 | CC(CNc1ncnc2scc(C)c12)c3ccccc3 | 5-methyl-N-(2-phenylpropyl)thieno[2,3- |
| | | d]pyrimidin-4-amine |
| EXAMPLE 120 | CC(Cc1ccncc1)Nc2ncnc3scc(C)c23 | 5-methyl-N-[1-(pyridin-4-yl)propan-2- |
| | | yl]thieno[2,3-d]pyrimidin-4-amine |
| EXAMPLE 121 | Cc1csc2ncnc(NCCc3ccc(O)cc3)c12 | 4-[2-({5-methylthieno[2,3-d]pyrimidin-4- |
| | | yl}amino)ethyl]phenol |
| EXAMPLE 122 | Cc1csc2ncnc(NCCOc3ccccc3)c12 | 5-methyl-N-(2-phenoxyethyl)thieno[2,3- |
| | | d]pyrimidin-4-amine |
| EXAMPLE 123 | COc1ccccc1CNc2ncnc3scc(C)c23 | N-[(2-methoxyphenyl)methyl]-5- |
| | | methylthieno[2,3-d]pyrimidin-4-amine |
| EXAMPLE 124 | COc1ccc(CNc2ncnc3scc(C)c23)cc1 | N-[(4-methoxyphenyl)methyl]-5- |
| | | methylthieno[2,3-d]pyrimidin-4-amine |
| EXAMPLE 125 | COc1cccc(CNc2ncnc3scc(C)c23)c1 | N-[(3-methoxyphenyl)methyl]-5- |
| | | methylthieno[2,3-d]pyrimidin-4-amine |
| EXAMPLE 126 | | This example has intentionally been left |
| | | blank |
| EXAMPLE 127 | Cc1csc2ncnc(NCCc3ccccc3F)c12 | N-[2-(2-fluorophenyl)ethyl]-5- |
| | | methylthieno[2,3-d]pyrimidin-4-amine |
| EXAMPLE 128 | | This example has intentionally been left |
| | | blank |
| EXAMPLE 129 | Cc1csc2ncnc(NCc3cccc(Cl)c3)c12 | N-[(3-chlorophenyl)methyl]-5- |
| | | methylthieno[2,3-d]pyrimidin-4-amine |
| EXAMPLE 130 | Cc1csc2ncnc(NCc3ccccc3Cl)c12 | N-[(2-chlorophenyl)methyl]-5- |
| | | methylthieno[2,3-d]pyrimidin-4-amine |
| EXAMPLE 131 | Cc1csc2ncnc(NCc3ccc(Cl)cc3)c12 | N-[(4-chlorophenyl)methyl]-5- |
| | | methylthieno[2,3-d]pyrimidin-4-amine |
| EXAMPLE 132 | Cc1csc2ncnc(NCCCN3CCCC3═O)c12 | 1-[3-({5-methylthieno[2,3-d]pyrimidin-4- |
| | | yl}amino)propyl]pyrrolidin-2-one |
| EXAMPLE 133 | Cc1csc2ncnc(NCc3ccc(F)cc3F)c12 | N-[(2,4-difluorophenyl)methyl]-5- |
| | | methylthieno[2,3-d]pyrimidin-4-amine |
| EXAMPLE 134 | Cc1csc2ncnc(NCc3ccc(F)c(F)c3)c12 | N-[(3,4-difluorophenyl)methyl]-5- |
| | | methylthieno[2,3-d]pyrimidin-4-amine |
| EXAMPLE 135 | Cc1csc2ncnc(NCc3cc(F)cc(F)c3)c12 | N-[(3,5-difluorophenyl)methyl]-5- |
| | | methylthieno[2,3-d]pyrimidin-4-amine |
| EXAMPLE 136 | Cc1csc2ncnc(NCCCN3CCOCC3)c12 | 5-methyl-N-[3-(morpholin-4- |
| | | yl)propyl]thieno[2,3-d]pyrimidin-4-amine |
| EXAMPLE 137 | CC(C)N(CCNc1ncnc2scc(C)c12)C(C)C | [2-({5-methylthieno[2,3-d]pyrimidin-4- |
| | | yl}amino)ethyl]bis(propan-2-yl))amine |
| EXAMPLE 138 | Cc1csc2ncnc(NC3CCCc4ccccc34)c12 | 5-methyl-N-(1,2,3,4-tetrahydronaphthalen- |
| | | 1-yl)thieno[2,3-d]pyrimidin-4-amine |
| EXAMPLE 139 | CC(CCc1ccccc1)Nc2ncnc3scc(C)c23 | 5-methyl-N-(4-phenylbutan-2- |
| | | yl)thieno[2,3-d]pyrimidin-4-amine |
| EXAMPLE 140 | CC(C)c1ccc(CNc2ncnc3scc(C)c23)cc1 | 5-methyl-N-{[4-(propan-2- |
| | | yl)phenyl]methyl}thieno[2,3-d]pyrimidin- |
| | | 4-amine |
| EXAMPLE 141 | Cc1csc2ncnc(NCc3ccc4OCOc4c3)c12 | N-(2H-1,3-benzodioxol-5-ylmethyl)-5- |
| | | methylthieno[2,3-d]pyrimidin-4-amine |
| EXAMPLE 142 | | This example has intentionally been left |
| | | blank |
| EXAMPLE 143 | Cc1csc2ncnc(N[C@H](CO)Cc3ccccc3)c12 | (2S)-2-({5-methylthieno[2,3-d]pyrimidin-4- |
| | | yl}amino)-3-phenylpropan-1-ol |
| EXAMPLE 144 | COc1ccc(CCNc2ncnc3scc(C)c23)cc1 | N-[2-(4-methoxyphenyl)ethyl]-5- |
| | | methylthieno[2,3-d]pyrimidin-4-amine |
| EXAMPLE 145 | COc1ccccc1CCNc2ncnc3scc(C)c23 | N-[2-(2-methoxyphenyl)ethyl]-5- |
| | | methylthieno[2,3-d]pyrimidin-4-amine |
| EXAMPLE 146 | CCOc1ccccc1CNc2ncnc3scc(C)c23 | N-[(2-ethoxyphenyl)methyl]-5- |
| | | methylthieno[2,3-d]pyrimidin-4-amine |
| EXAMPLE 147 | COc1ccc(Nc2ncnc3scc(C)c23)cc1OC | N-(3,4-dimethoxyphenyl)-5- |
| | | methylthieno[2,3-d]pyrimidin-4-amine |
| EXAMPLE 148 | Cc1csc2ncnc(NCCc3ccccc3Cl)c12 | N-[2-(2-chlorophenyl)ethyl]-5- |
| | | methylthieno[2,3-d]pyrimidin-4-amine |
| EXAMPLE 149 | Cc1csc2ncnc(NCCc3cccc(Cl)c3)c12 | N-[2-(3-chlorophenyl)ethyl]-5- |
| | | methylthieno[2,3-d]pyrimidin-4-amine |
| EXAMPLE 150 | Cc1csc2ncnc(NC3CC(C)(C)NC(C)(C)C3)c12 | 2,2,6,6-tetramethyl-N-{5-methylthieno[2,3- |
| | | d]pyrimidin-4-yl}piperidin-4-amine |
| EXAMPLE 151 | CCN(CC)CCCC(C)Nc1ncnc2scc(C)c12 | diethyl[4-({5-methylthieno[2,3- |
| | | d]pyrimidin-4-yl}amino)pentyl]amine |
| EXAMPLE 152 | Cc1csc2ncnc(NCc3ccc(F)c(Cl)c3)c12 | N-[(3-chloro-4-fluorophenyl)methyl]-5- |
| | | methylthieno[2,3-d]pyrimidin-4-amine |
| EXAMPLE 153 | Cc1csc2ncnc(N(CCC#N)Cc3ccccc3)c12 | 3-[benzyl({5-methylthieno[2,3- |
| | | d]pyrimidin-4-yl})amino]propanenitrile |
| EXAMPLE 154 | Cc1csc2ncnc(NCCNC(═O)OC(C)(C)C)c12 | tert-butyl N-[2-({5-methylthieno[2,3- |
| | | d]pyrimidin-4-yl}amino)ethyl]carbamate |
| EXAMPLE 155 | Cc1csc2ncnc(NCCc3c[nH]c4ccccc34)c12 | N-[2-(1H-indol-3-yl)ethyl]-5- |
| | | methylthieno[2,3-d]pyrimidin-4-amine |
| EXAMPLE 156 | Cc1csc2ncnc(NCc3ccc(cc3)C(C)(C)C)c12 | N-[(4-tert-butylphenyl)methyl]-5- |
| | | methylthieno[2,3-d]pyrimidin-4-amine |
| EXAMPLE 157 | CN(CCCNc1ncnc2scc(C)c12)c3ccccc3 | N-methyl-N-[3-({5-methylthieno[2,3- |
| | | d]pyrimidin-4-yl}amino)propyl]aniline |
| EXAMPLE 158 | Cc1csc2ncnc(NCC3(O)CCCCC3)c12 | 1-[({5-methylthieno[2,3-d]pyrimidin-4- |
| | | yl}amino)methyl]cyclohexan-1-ol |
| EXAMPLE 159 | COc1cc(CNc2ncnc3scc(C)c23)cc(OC)c1 | N-[(3,5-dimethoxyphenyl)methyl]-5- |
| | | methylthieno[2,3-d]pyrimidin-4-amine |
| EXAMPLE 160 | COc1ccc(CNc2ncnc3scc(C)c23)cc1OC | N-[(3,4-dimethoxyphenyl)methyl]-5- |
| | | methylthieno[2,3-d]pyrimidin-4-amine |
| EXAMPLE 161 | Cc1csc2ncnc(NCC(═O)c3ccccc3)c12 | 2-({5-methylthieno[2,3-d]pyrimidin-4- |
| | | yl}amino)-1-phenylethan-1-one |
| EXAMPLE 162 | CCOC(═O)N1CCC(CC1)Nc2ncnc3scc(C)c23 | ethyl 4-({5-methylthieno[2,3-d]pyrimidin- |
| | | 4-yl}amino)piperidine-1-carboxylate |
| EXAMPLE 163 | Cc1csc2ncnc(NCCCNC(═O)OC(C)(C)C)c12 | tert-butyl N-[3-({5-methylthieno[2,3- |
| | | d]pyrimidin-4-yl}amino)propyl]carbamate |
| EXAMPLE 164 | Cc1csc2ncnc(NCc3ccc(cc3)C(F)(F)F)c12 | 5-methyl-N-{[4- |
| | | (trifluoromethyl)phenyl]methyl}thieno[2,3- |
| | | d]pyrimidin-4-amine |
| EXAMPLE 165 | Cc1csc2ncnc(NCc3cccc(c3)C(F)(F)F)c12 | 5-methyl-N-{[3- |
| | | (trifluoromethyl)phenyl]methyl}thieno[2,3- |
| | | d]pyrimidin-4-amine |
| EXAMPLE 166 | Cc1csc2ncnc(NCc3ccc(Cl)c(Cl)c3)c12 | N-[(3,4-dichlorophenyl)methyl]-5- |
| | | methylthieno[2,3-d]pyrimidin-4-amine |
| EXAMPLE 167 | Cc1csc2ncnc(NCc3ccc(Cl)cc3Cl)c12 | N-[(2,4-dichlorophenyl)methyl]-5- |
| | | methylthieno[2,3-d]pyrimidin-4-amine |
| EXAMPLE 168 | Cc1csc2ncnc(NC3CCN(C3)Cc4ccccc4)c12 | 1-benzyl-N-{5-methylthieno[2,3- |
| | | d]pyrimidin-4-yl}pyrrolidin-3-amine |
| EXAMPLE 169 | COc1ccc(OC)c(CCNc2ncnc3scc(C)c23)c1 | N-[2-(2,5-dimethoxyphenyl)ethyl]-5- |
| | | methylthieno[2,3-d]pyrimidin-4-amine |
| EXAMPLE 170 | CN(C)c1ccc(CNc2ncnc3scc(C)c23)cc1 | N-{[4-(dimethylamino)phenyl]methyl}-5- |
| | | methylthieno[2,3-d]pyrimidin-4-amine |
| EXAMPLE 171 | COc1ccc(CNc2ncnc3scc(C)c23)cc1O | 2-methoxy-5-[({5-methylthieno[2,3- |
| | | d]pyrimidin-4-yl}amino)methyl]phenol |
| EXAMPLE 172 | Cc1csc2ncnc(NCc3ccccc3OC(F)(F)F)c12 | 5-methyl-N-{[2- |
| | | (trifluoromethoxy)phenyl]methyl}thieno[2, |
| | | 3-d]pyrimidin-4-amine |
| EXAMPLE 173 | Cc1csc2ncnc(NCCc3ccc(cc3)S(N)(═O)═O)c12 | 4-[2-({5-methylthieno[2,3-d]pyrimidin-4- |
| | | yl}amino)ethyl]benzene-1-sulfonamide |
| EXAMPLE 174 | COc1ccc(cc1)C(═O)CNc2ncnc3scc(C)c23 | 1-(4-methoxyphenyl)-2-({5- |
| | | methylthieno[2,3-d]pyrimidin-4- |
| | | yl}amino)ethan-1-one |
| EXAMPLE 175 | Cc1csc2ncnc(NCCc3ccc4OCOc4c3)c12 | N-[2-(2H-1,3-benzodioxol-5-yl)ethyl]-5- |
| | | methylthieno[2,3-d]pyrimidin-4-amine |
| EXAMPLE 176 | Cc1csc2ncnc(NCCC(c3ccccc3)c4ccccc4)c12 | N-(3,3-diphenylpropyl)-5- |
| | | methylthieno[2,3-d]pyrimidin-4-amine |
| EXAMPLE 177 | Cc1ccc(cc1)S(═O)(═O)NCCNc2ncnc3scc(C)c23 | 4-methyl-N-[2-({5-methylthieno[2,3- |
| | | d]pyrimidin-4-yl}amino)ethyl]benzene-1- |
| | | sulfonamide |
| EXAMPLE 178 | CN(C)c1ncnc2scc(C)c12 | N,N,5-trimethylthieno[2,3-d]pyrimidin-4- |
| | | amine |
| EXAMPLE 179 | CCN(C)c1ncnc2scc(C)c12 | N-ethyl-N,5-dimethylthieno[2,3- |
| | | d]pyrimidin-4-amine |
| EXAMPLE 180 | CN(CC#C)c1ncnc2scc(C)c12 | N,5-dimethyl-N-(prop-2-yn-1- |
| | | yl)thieno[2,3-d]pyrimidin-4-amine |
| EXAMPLE 181 | CCN(CC)c1ncnc2scc(C)c12 | N,N-diethyl-5-methylthieno[2,3- |
| | | d]pyrimidin-4-amine |
| EXAMPLE 182 | CCCN(C)c1ncnc2scc(C)c12 | N,5-dimethyl-N-propylthieno[2,3- |
| | | d]pyrimidin-4-amine |
| EXAMPLE 183 | CN(CCO)c1ncnc2scc(C)c12 | 2-[methyl({5-methylthieno[2,3- |
| | | d]pyrimidin-4-yl})amino]ethan-1-ol |
| EXAMPLE 184 | CN(CCC#N)c1ncnc2scc(C)c12 | 3-[methyl({5-methylthieno[2,3- |
| | | d]pyrimidin-4-yl})amino]propanenitrile |
| EXAMPLE 185 | CC(C)CN(C)c1ncnc2scc(C)c12 | N,5-dimethyl-N-(2- |
| | | methylpropyl)thieno[2,3-d]pyrimidin-4- |
| | | amine |
| EXAMPLE 186 | CCN(C(C)C)c1ncnc2scc(C)c12 | N-ethyl-5-methyl-N-(propan-2- |
| | | yl)thieno[2,3-d]pyrimidin-4-amine |
| EXAMPLE 187 | CCCCN(C)c1ncnc2scc(C)c12 | N-butyl-N,5-dimethylthieno[2,3- |
| | | d]pyrimidin-4-amine |
| EXAMPLE 188 | CCN(CCO)c1ncnc2scc(C)c12 | 2-[ethyl({5-methylthieno[2,3-d]pyrimidin- |
| | | 4-yl})amino]ethan-1-ol |
| EXAMPLE 189 | Cc1csc2ncnc(N3CCSC3)c12 | 3-{5-methylthieno[2,3-d]pyrimidin-4-yl}- |
| | | 1,3-thiazolidine |
| EXAMPLE 190 | CC1CCCCN1c2ncnc3scc(C)c23 | 2-methyl-1-{5-methylthieno[2,3- |
| | | d]pyrimidin-4-yl}piperidine |
| EXAMPLE 191 | CC1CCN(CC1)c2ncnc3scc(C)c23 | 4-methyl-1-{5-methylthieno[2,3- |
| | | d]pyrimidin-4-yl}piperidine |
| EXAMPLE 192 | CN1CCN(CC1)c2ncnc3scc(C)c23 | 1-methyl-4-{5-methylthieno[2,3- |
| | | d]pyrimidin-4-yl}piperazine |
| EXAMPLE 193 | Cc1csc2ncnc(N3CCCC3CO)c12 | (1-{5-methylthieno[2,3-d]pyrimidin-4- |
| | | yl}pyrrolidin-2-yl)methanol |
| EXAMPLE 194 | Cc1csc2ncnc(N3CCC[C@@H]3CO)c12 | [(2R)-1-{5-methylthieno[2,3-d]pyrimidin- |
| | | 4-yl}pyrrolidin-2-yl]methanol |
| EXAMPLE 195 | CCCCN(CC)c1ncnc2scc(C)c12 | N-butyl-N-ethyl-5-methylthieno[2,3- |
| | | d]pyrimidin-4-amine |
| EXAMPLE 196 | CCCN(CCC)c1ncnc2scc(C)c12 | 5-methyl-N,N-dipropylthieno[2,3- |
| | | d]pyrimidin-4-amine |
| EXAMPLE 197 | Cc1csc2ncnc(N3CCSCC3)c12 | 4-{5-methylthieno[2,3-d]pyrimidin-4- |
| | | yl}thiomorpholine |
| EXAMPLE 198 | Cc1csc2ncnc(N(CCO)CCO)c12 | 2-[(2-hydroxyethyl)({5-methylthieno[2,3- |
| | | d]pyrimidin-4-yl})amino]ethan-1-ol |
| EXAMPLE 199 | CC1CC(C)CN(C1)c2ncnc3scc(C)c23 | 3,5-dimethyl-1-{5-methylthieno[2,3- |
| | | d]pyrimidin-4-yl}piperidine |
| EXAMPLE 200 | CN(C1CCCCC1)c2ncnc3scc(C)c23 | N-cyclohexyl-N,5-dimethylthieno[2,3- |
| | | d]pyrimidin-4-amine |
| EXAMPLE 201 | CC1CCCC(C)N1c2ncnc3scc(C)c23 | 2,6-dimethyl-1-{5-methylthieno[2,3- |
| | | d]pyrimidin-4-yl}piperidine |
| EXAMPLE 202 | CN(C)C1CCN(C1)c2ncnc3scc(C)c23 | N,N-dimethyl-1-{5-methylthieno[2,3- |
| | | d]pyrimidin-4-yl}pyrrolidin-3-amine |
| EXAMPLE 203 | C[C@@H]1CN(C[C@H](C)N1)c2ncnc3scc(C)c23 | (3R,5S)-3,5-dimethyl-1-{5- |
| | | methylthieno[2,3-d]pyrimidin-4- |
| | | yl}piperazine |
| EXAMPLE 204 | Cc1csc2ncnc(N3CCCC(CO)C3)c12 | (1-{5-methylthieno[2,3-d]pyrimidin-4- |
| | | yl}piperidin-3-yl)methanol |
| EXAMPLE 205 | COC[C@@H]1CCCN1c2ncnc3scc(C)c23 | (2S)-2-(methoxymethyl)-1-{5- |
| | | methylthieno[2,3-d]pyrimidin-4- |
| | | yl}pyrrolidine |
| EXAMPLE 206 | Cc1csc2ncnc(N3CCCCC3CO)c12 | (1-{5-methylthieno[2,3-d]pyrimidin-4- |
| | | yl}piperidin-2-yl)methanol |
| EXAMPLE 207 | CCN(CCCCO)c1ncnc2scc(C)c12 | 4-[ethyl({5-methylthieno[2,3-d]pyrimidin- |
| | | 4-yl})amino]butan-1-ol |
| EXAMPLE 208 | Cc1csc2ncnc(N3Cc4ccccc4C3)c12 | 2-{5-methylthieno[2,3-d]pyrimidin-4-yl}- |
| | | 2,3-dihydro-1H-isoindole |
| EXAMPLE 209 | CN(Cc1ccccc1)c2ncnc3scc(C)c23 | N-benzyl-N,5-dimethylthieno[2,3- |
| | | d]pyrimidin-4-amine |
| EXAMPLE 210 | CCN(C1CCCCC1)c2ncnc3scc(C)c23 | N-cyclohexyl-N-ethyl-5-methylthieno[2,3- |
| | | d]pyrimidin-4-amine |
| EXAMPLE 211 | Cc1csc2ncnc(N3CCC(CC3)C(N)═O)c12 | 1-{5-methylthieno[2,3-d]pyrimidin-4- |
| | | yl}piperidine-4-carboxamide |
| EXAMPLE 212 | CC(═O)N[C@H]1CCN(C1)c2ncnc3scc(C)c23 | N-[(3S)-1-{5-methylthieno[2,3- |
| | | d]pyrimidin-4-yl}pyrrolidin-3-yl]acetamide |
| EXAMPLE 213 | Cc1csc2ncnc(N3CCCC(C3)C(N)═O)c12 | 1-{5-methylthieno[2,3-d]pyrimidin-4- |
| | | yl}piperidine-3-carboxamide |
| EXAMPLE 214 | CC(═O)N[C@H]1CCN(C1)c2ncnc3scc(C)c23 | N-[(3S)-1-{5-methylthieno[2,3- |
| | | d]pyrimidin-4-yl}pyrrolidin-3-yl]acetamide |
| EXAMPLE 215 | Cc1csc2ncnc(N3CCCCC3CCO)c12 | 2-(1-{5-methylthieno[2,3-d]pyrimidin-4- |
| | | yl}piperidin-2-yl)ethan-1-ol |
| EXAMPLE 216 | Cc1csc2ncnc(N3CCc4ccccc4C3)c12 | 2-{5-methylthieno[2,3-d]pyrimidin-4-yl}- |
| | | 1,2,3,4-tetrahydroisoquinoline |
| EXAMPLE 217 | CCN(Cc1ccccc1)c2ncnc3scc(C)c23 | N-benzyl-N-ethyl-5-methylthieno[2,3- |
| | | d]pyrimidin-4-amine |
| EXAMPLE 218 | CN(CCc1ccccc1)c2ncnc3scc(C)c23 | N,5-dimethyl-N-(2-phenylethyl)thieno[2,3- |
| | | d]pyrimidin-4-amine |
| EXAMPLE 219 | CCN(Cc1ccccc1)c2ncnc3scc(C)c23 | N-benzyl-N-ethyl-5-methylthieno[2,3- |
| | | d]pyrimidin-4-amine |
| EXAMPLE 220 | CN(CCc1ccccn1)c2ncnc3scc(C)c23 | N,5-dimethyl-N-[2-(pyridin-2- |
| | | yl)ethyl]thieno[2,3-d]pyrimidin-4-amine |
| EXAMPLE 221 | CN(CCc1ccccn1)c2ncnc3scc(C)c23 | N,5-dimethyl-N-[2-(pyridin-2- |
| | | yl)ethyl]thieno[2,3-d]pyrimidin-4-amine |
| EXAMPLE 222 | Cc1csc2ncnc(N3CCCC4CCCCC34)c12 | 1-{5-methylthieno[2,3-d]pyrimidin-4-yl}- |
| | | decahydroquinoline |
| EXAMPLE 223 | Cc1csc2ncnc(N(CC═C)C3 CCCCC3)c12 | N-cyclohexyl-5-methyl-N-(prop-2-en-1- |
| | | yl)thieno[2,3-d]pyrimidin-4-amine |
| EXAMPLE 224 | COC(═O)C1CCN(CC1)c2ncnc3scc(C)c23 | methyl 1-{5-methylthieno[2,3-d]pyrimidin- |
| | | 4-yl}piperidine-4-carboxylate |
| EXAMPLE 225 | CC(C)N(Cc1ccccc1)c2ncnc3scc(C)c23 | N-benzyl-5-methyl-N-(propan-2- |
| | | yl)thieno[2,3-d]pyrimidin-4-amine |
| EXAMPLE 226 | Cc1csc2ncnc(N(CCO)Cc3ccccc3)c12 | 2-[benzyl({5-methylthieno[2,3- |
| | | d]pyrimidin-4-yl})amino]ethan-1-ol |
| EXAMPLE 227 | CN(CC(O)c1ccccc1)c2ncnc3scc(C)c23 | 2-[methyl({5-methylthieno[2,3- |
| | | d]pyrimidin-4-yl})amino]-1-phenylethan-1- |
| | | ol |
| EXAMPLE 228 | Cc1csc2ncnc(N3CCC(O)(O)CC3)c12 | 1-{5-methylthieno[2,3-d]pyrimidin-4- |
| | | yl}piperidine-4,4-diol |
| EXAMPLE 229 | CCOC(═O)C1CCCN(C1)c2ncnc3scc(C)c23 | ethyl 1-{5-methylthieno[2,3-d]pyrimidin-4- |
| | | yl}piperidine-3-carboxylate |
| EXAMPLE 230 | CCOC(═O)N1CCN(CC1)c2ncnc3sccC)c23 | ethyl 4-{5-methylthieno[2,3-d]pyrimidin-4- |
| | | (yl}piperazine-1-carboxylate |
| EXAMPLE 231 | Cc1csc2ncnc(N(CCC#N)Cc3ccccc3)c12 | 3-[benzyl({5-methylthieno[2,3- |
| | | d]pyrimidin-4-yl})amino]propanenitrile |
| EXAMPLE 232 | Cc1csc2ncnc(N(CCC#N)Cc3cccnc3)c12 | 3-({5-methylthieno[2,3-d]pyrimidin-4- |
| | | yl}(pyridin-3- |
| | | ylmethyl)amino)propanenitrile |
| EXAMPLE 233 | Cc1csc2ncnc(N3CCN(CC3)c4ccccc4)c12 | 1-{5-methylthieno[2,3-d]pyrimidin-4-yl}- |
| | | 4-phenylpiperazine |
| EXAMPLE 234 | Cc1csc2ncnc(N3CCN(CC3)c4ccccn4)c12 | 1-{5-methylthieno[2,3-d]pyrimidin-4-yl}- |
| | | 4-(pyridin-2-yl)piperazine |
| EXAMPLE 235 | CCCCN(Cc1ccccc1)c2ncnc3scc(C)c23 | N-benzyl-N-butyl-5-methylthieno[2,3- |
| | | d]pyrimidin-4-amine |
| EXAMPLE 236 | C[C@@H](N(CCO)c1ncnc2scc(C)c12)c3ccccc3 | 2-({5-methylthieno[2,3-d]pyrimidin-4- |
| | | yl}[(1R)-1-phenylethyl]amino)ethan-1-ol |
| EXAMPLE 237 | Cc1csc2ncnc(N(CCCO)Cc3ccccn3)c12 | 3-({5-methylthieno[2,3-d]pyrimidin-4- |
| | | yl}(pyridin-2-ylmethyl)amino)propan-1-ol |
| EXAMPLE 238 | CN(CC(O)c1ccc(O)cc1)c2ncnc3scc(C)c23 | 4-{1-hydroxy-2-[methyl({5- |
| | | methylthieno[2,3-d]pyrimidin-4- |
| | | yl})amino]ethyl}phenol |
| EXAMPLE 239 | Cc1csc2ncnc(N3CCN(CC3)C4CCCCC4)c12 | 1-cyclohexyl-4-{5-methylthieno[2,3- |
| | | d]pyrimidin-4-yl}piperazine |
| EXAMPLE 240 | Cc1csc2ncnc(N3CCC(CC3)Cc4ccccc4)c12 | 4-benzyl-1-{5-methylthieno[2,3- |
| | | d]pyrimidin-4-yl}piperidine |
| EXAMPLE 241 | Cc1csc2ncnc(N3CCN(CC3)Cc4ccccc4)c12 | 1-benzyl-4-{5-methylthieno[2,3- |
| | | d]pyrimidin-4-yl}piperazine |
| EXAMPLE 242 | Cc1csc2ncnc(N3CCCN(CC3)Cc4ccccc4)c12 | 1-benzyl-4-{5-methylthieno[2,3- |
| | | d]pyrimidin-4-yl}-1,4-diazepane |
| EXAMPLE 243 | Cc1csc2ncnc(N3CCN(CC3)c4ccc(O)cc4)c12 | 4-(4-{5-methylthieno[2,3-d]pyrimidin-4- |
| | | yl}piperazin-1-yl)phenol |
| EXAMPLE 244 | CN(C)CCN(Cc1ccccc1)c2ncnc3scc(C)c23 | N-benzyl-N-[2-(dimethylamino)ethyl]-5- |
| | | methylthieno[2,3-d]pyrimidin-4-amine |
| EXAMPLE 245 | Cc1csc2ncnc(N3CCN(CC3)c4ccccc4F)c12 | 1-(2-fluorophenyl)-4-{5-methylthieno[2,3- |
| | | d]pyrimidin-4-yl}piperazine |
| EXAMPLE 246 | Cc1csc2ncnc(N3CCN(CC3)c4ccc(F)cc4)c12 | 1-(4-fluorophenyl)-4-{5-methylthieno[2,3- |
| | | d]pyrimidin-4-yl}piperazine |
| EXAMPLE 247 | Cc1csc2ncnc(N3CCN(CC3)CC4CCCCC4)c12 | 1-(cyclohexylmethyl)-4-{5- |
| | | methylthieno[2,3-d]pyrimidin-4- |
| | | yl}piperazine |
| EXAMPLE 248 | CN(C[C@H](O)c1ccc(O)c(O)c1)c2ncnc3scc(C)c23 | 4-[(1R)-1-hydroxy-2-[methyl({5- |
| | | methylthieno[2,3-d]pyrimidin-4- |
| | | yl})amino]ethyl]benzene-1,2-diol |
| EXAMPLE 249 | CCOC(═O)CC1N(CCNC1═O)c2ncnc3scc(C)c23 | ethyl 2-(1-{5-methylthieno[2,3- |
| | | d]pyrimidin-4-yl}-3-oxopiperazin-2- |
| | | yl)acetate |
| EXAMPLE 250 | Cc1csc2ncnc(N3CCN(CC3)C(═O)OC(C)(C)C)c12 | tert-butyl 4-{5-methylthieno[2,3- |
| | | d]pyrimidin-4-yl}piperazine-1-carboxylate |
| EXAMPLE 251 | Cc1csc2ncnc(N3CCC(C3)NC(═O)OC(C)(C)C)c12 | tert-butyl N-(1-{5-methylthieno[2,3- |
| | | d]pyrimidin-4-yl}pyrrolidin-3-yl)carbamate |
| EXAMPLE 252 | Cc1csc2ncnc(N3CCN(CC3)c4ccccc4C#N)c12 | 2-(4-{5-methylthieno[2,3-d]pyrimidin-4- |
| | | yl}piperazin-1-yl)benzonitrile |
| EXAMPLE 253 | Cc1csc2ncnc(N3CCN(CC3)c4ccc(cn4)C#N)c12 | 6-(4-{5-methylthieno[2,3-d]pyrimidin-4- |
| | | yl}piperazin-1-yl)pyridine-3-carbonitrile |
| EXAMPLE 254 | Cc1cccc(N2CCN(CC2)c3ncnc4scc(C)c34)c1C | 1-(2,3-dimethylphenyl)-4-{5- |
| | | methylthieno[2,3-d]pyrimidin-4- |
| | | yl}piperazine |
| EXAMPLE 255 | CC1CN(CCN1c2cccc(C)c2)c3ncnc4scc(C)c34 | 2-methyl-1-(3-methylphenyl)-4-{5- |
| | | methylthieno[2,3-d]pyrimidin-4- |
| | | yl}piperazine |
| EXAMPLE 256 | Cc1csc2ncnc(N3CCN(CC3)CCc4ccccc4)c12 | 1-{5-methylthieno[2,3-d]pyrimidin-4-yl}- |
| | | 4-(2-phenylethyl)piperazine |
| EXAMPLE 257 | Cc1ccc(cc1C)N2CCN(CC2)c3ncnc4scc(C)c34 | 1-(3,4-dimethylphenyl)-4-{5- |
| | | methylthieno[2,3-d]pyrimidin-4- |
| | | yl}piperazine |
| EXAMPLE 258 | Cc1ccc(N2CCN(CC2)c3ncnc4scc(C)c34)c(C)c1 | 1-(2,4-dimethylphenyl)-4-{5- |
| | | methylthieno[2,3-d]pyrimidin-4- |
| | | yl}piperazine |
| EXAMPLE 259 | Cc1ccc(C)c(c1)N2CCN(CC2)c3ncnc4scc(C)c34 | 1-(2,5-dimethylphenyl)-4-{5- |
| | | methylthieno[2,3-d]pyrimidin-4- |
| | | yl}piperazine |
| EXAMPLE 260 | Cc1cccc(N2CCN(CC2)c3ncnc4scc(C)c34)c1C | 1-(2,3-dimethylphenyl)-4-{5- |
| | | methylthieno[2,3-d]pyrimidin-4- |
| | | yl}piperazine |
| EXAMPLE 261 | COc1ccc(cc1)N2CCN(CC2)c3ncnc4scc(C)c34 | 1-(4-methoxyphenyl)-4-{5- |
| | | methylthieno[2,3-d]pyrimidin-4- |
| | | yl}piperazine |
| EXAMPLE 262 | COc1ccccc1N2CCN(CC2)c3ncnc4scc(C)c34 | 1-(2-methoxyphenyl)-4-{5- |
| | | methylthieno[2,3-d]pyrimidin-4- |
| | | yl}piperazine |
| EXAMPLE 263 | COc1cccc(c1)N2CCN(CC2)c3ncnc4scc(C)c34 | 1-(3-methoxyphenyl)-4-{5- |
| | | methylthieno[2,3-d]pyrimidin-4- |
| | | yl}piperazine |
| EXAMPLE 264 | COc1ccc(CCN(C)c2ncnc3scc(C)c23)cc1OC | N-[2-(3,4-dimethoxyphenyl)ethyl]-N,5- |
| | | dimethylthieno[2,3-d]pyrimidin-4-amine |
| EXAMPLE 265 | Cc1csc2ncnc(N3CCN(CC3)c4cccc(C1)c4)c12 | 1-(3-chlorophenyl)-4-{5-methylthieno[2,3- |
| | | d]pyrimidin-4-yl}piperazine |
| EXAMPLE 266 | Cc1csc2ncnc(N3CCN(CC3)c4ccc(C1)cc4)c12 | 1-(4-chlorophenyl)-4-{5-methylthieno[2,3- |
| | | d]pyrimidin-4-yl}piperazine |
| EXAMPLE 267 | Cc1csc2ncnc(N3CCN(CC3)c4ccc(F)cc4F)c12 | 1-(2,4-difluorophenyl)-4-{5- |
| | | methylthieno[2,3-d]pyrimidin-4- |
| | | yl}piperazine |
| EXAMPLE 268 | Cc1csc2ncnc(N(Cc3cccnc3)Cc4cccnc4)c12 | 5-methyl-N,N-bis(pyridin-3- |
| | | ylmethyl)thieno[2,3-d]pyrimidin-4-amine |
| EXAMPLE 269 | Cc1csc2ncnc(N3CCN(CCN4CCOCC4)CC3)c12 | 4-[2-(4-{5-methylthieno[2,3-d]pyrimidin-4- |
| | | yl}piperazin-1-yl)ethyl]morpholine |
| EXAMPLE 270 | CN(C1CCN(C1)c2ncnc3scc(C)c23)C(═O)OC(C)(C)C | tert-butyl N-methyl-N-(1-{5- |
| | | methylthieno[2,3-d]pyrimidin-4- |
| | | yl}pyrrolidin-3-yl)carbamate |
| EXAMPLE 271 | CC(═O)c1ccc(cc1)N2CCN(CC2)c3ncnc4scc(C)c34 | 1-[4-(4-{5-methylthieno[2,3-d]pyrimidin-4- |
| | | yl}piperazin-1-yl)phenyl]ethan-1-one |
| EXAMPLE 272 | CCN(CC)CCN(Cc1ccccc1)c2ncnc3scc(C)c23 | N-benzyl-N-[2-(diethylamino)ethyl]-5- |
| | | methylthieno[2,3-d]pyrimidin-4-amine |
| EXAMPLE 273 | Cc1csc2ncnc(N3CCN(CC3)c4ccc(cc4)[N+]([O−])═O)c12 | 1-{5-methylthieno[2,3-d]pyrimidin-4-yl}- |
| | | 4-(4-nitrophenyl)piperazine |
| EXAMPLE 274 | CN(Cc1cccc2ccccc12)c3ncnc4scc(C)c34 | N,5-dimethyl-N-(naphthalen-1- |
| | | ylmethyl)thieno[2,3-d]pyrimidin-4-amine |
| EXAMPLE 275 | Cc1ccc(C1)cc1N2CCN(CC2)c3ncnc4scc(C)c34 | 1-(5-chloro-2-methylphenyl)-4-{5- |
| | | methylthieno[2,3-d]pyrimidin-4- |
| | | yl}piperazine |
| EXAMPLE 276 | COc1cc(CN(C)c2ncnc3scc(C)c23)cc(OC)c1OC | N,5-dimethyl-N-[(3,4,5- |
| | | trimethoxyphenyl)methyl]thieno[2,3- |
| | | d]pyrimidin-4-amine |
| EXAMPLE 277 | Cc1csc2ncnc(N(CCc3ccccc3)Cc4ccccc4)c12 | N-benzyl-5-methyl-N-(2- |
| | | phenylethyl)thieno[2,3-d]pyrimidin-4- |
| | | amine |
| EXAMPLE 278 | CN(C(Cc1ccccc1)c2ccccc2)c3ncnc4scc(C)c34 | N-(1,2-diphenylethyl)-N,5- |
| | | dimethylthieno[2,3-d]pyrimidin-4-amine |
| EXAMPLE 279 | Cc1csc2ncnc(N(CCc3ccccc3)Cc4ccccc4)c12 | N-benzyl-5-methyl-N-(2- |
| | | phenylethyl)thieno[2,3-d]pyrimidin-4- |
| | | amine |
| EXAMPLE 280 | Cc1csc2ncnc(N3CCC(O)(CC3)c4ccc(C1)cc4)c12 | 4-(4-chlorophenyl)-1-{5-methylthieno[2,3- |
| | | d]pyrimidin-4-yl}piperidin-4-ol |
| EXAMPLE 281 | Cc1csc2ncnc(N(CC#C)Cc3ccc(C1)cc3C1)c12 | N-[(2,4-dichlorophenyl)methyl]-5-methyl- |
| | | N-(prop-2-yn-1-yl)thieno[2,3-d]pyrimidin- |
| | | 4-amine |
| EXAMPLE 282 | CCN(C(Cc1ccccc1)c2ccco2)c3ncnc4scc(C)c34 | N-ethyl-N-[1-(furan-2-yl)-2-phenylethyl]- |
| | | 5-methylthieno[2,3-d]pyrimidin-4-amine |
| EXAMPLE 283 | Cc1csc2ncnc(N3CCN(C[C@H]3CO)C(═O)OC(C)(C)C)c12 | tert-butyl (3S)-3-(hydroxymethyl)-4-{5- |
| | | methylthieno[2,3-d]pyrimidin-4- |
| | | yl}piperazine-1-carboxylate |
| EXAMPLE 284 | Cc1csc2ncnc(N3CCN(CC3)c4ccc(cc4)C(C)(C)C)c12 | 1-(4-tert-butylphenyl)-4-{5- |
| | | methylthieno[2,3-d]pyrimidin-4- |
| | | yl}piperazine |
| EXAMPLE 285 | Cc1csc2ncnc(N3CCN(CC3)Cc4ccc5OCOc5c4)c12 | 1-(2H-1,3-benzodioxol-5-ylmethyl)-4-{5- |
| | | methylthieno[2,3-d]pyrimidin-4- |
| | | yl}piperazine |
| EXAMPLE 286 | COc1cc2CCN(Cc2cc1OC)c3ncnc4scc(C)c34 | 6,7-dimethoxy-2-{5-methylthieno[2,3- |
| | | d]pyrimidin-4-yl}-1,2,3,4- |
| | | tetrahydroisoquinoline |
| EXAMPLE 287 | Cc1csc2ncnc(N3CCN(CC3)c4ccc(cc4)C(F)(F)F)c12 | 1-{5-methylthieno[2,3-d]pyrimidin-4-yl}- |
| | | 4-[4-(trifluoromethyl)phenyl]piperazine |
| EXAMPLE 288 | Cc1csc2ncnc(N3CCN(CC3)c4cccc(c4)C(F)(F)F)c12 | 1-{5-methylthieno[2,3-d]pyrimidin-4-yl}- |
| | | 4-[3-(trifluoromethyl)phenyl]piperazine |

### EXAMPLE 289

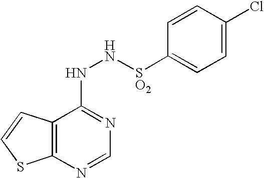

### 4-chloro-N′-(thieno[2,3-d]pyrimidin-4-yl)benzenesulfonohydrazide

### EXAMPLE 290

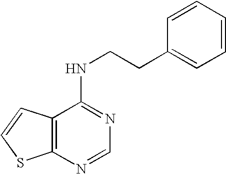

### N-phenethylthieno[2,3-d]pyrimidin-4-amine

### EXAMPLE 291

This example has intentionally been left blank

### EXAMPLE 292

This example has intentionally been left blank

### EXAMPLE 293

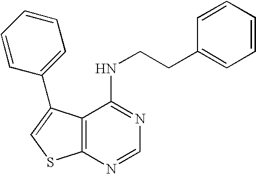

### N-(4-chlorophenethyl)-5-phenylthieno[2,3-d]pyrimidin-4-amine

### EXAMPLE 294

This example has intentionally been left blank

### EXAMPLE 295

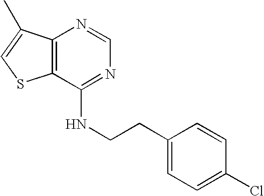

### N-(4-chlorophenethyl)-7-methylthieno[3,2-d]pyrimidin-4-amine

### EXAMPLE 296

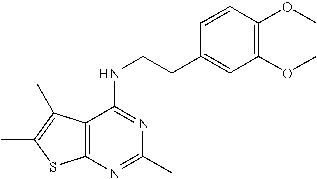

### N-(3,4-dimethoxyphenethyl)-2,5,6-trimethylthieno[2,3-d]pyrimidin-4-amine

### EXAMPLE 297

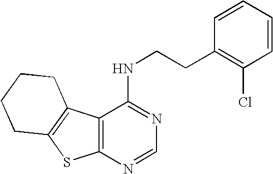

### EXAMPLE 298

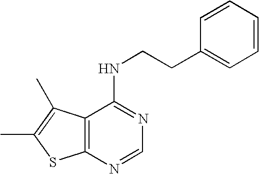

### N-(2-methoxyphenethyl)-5,6-dimethylthieno[2,3-d]pyrimidin-4-amine

### EXAMPLE 299

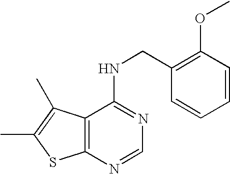

### N-(2-methoxyphenethyl)-5,6-dimethylthieno[2,3-d]pyrimidin-4-amine

### EXAMPLE 300

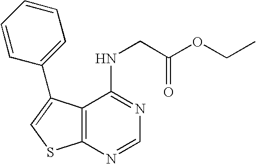

### Ethyl 2-(5-phenylthieno[2,3-d]pyrimidin-4-ylamino)acetate

### EXAMPLE 301

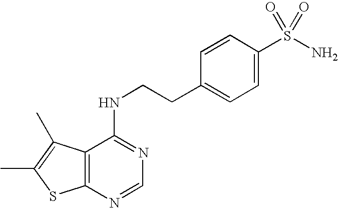

### 4-(2-(5,6-dimethylthieno[2,3-d]pyrimidin-4-ylamino)ethyl)benzenesulfonamide

### EXAMPLE 302

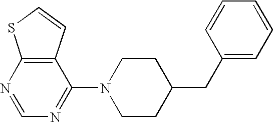

### 4-(4-benzylpiperidin-1-yl)thieno[2,3-d]pyrimidine

### EXAMPLE 303

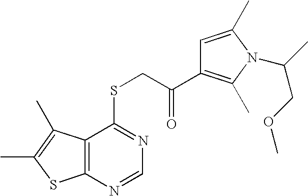

### 2-(5,6-dimethylthieno[2,3-d]pyrimidin-4-ylthio)-1-(1-(1-methoxypropan-2-yl)-2,5-dimethyl-1H-pyrrol-3-yl)ethanone

### EXAMPLE 304

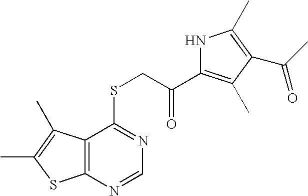

### 1-(4-acetyl-3,5-dimethyl-1H-pyrrol-2-yl)-2-(5,6-dimethylthieno[2,3-d]pyrimidin-4-ylthio)ethanone

### EXAMPLE 305

This example has intentionally been left blank

### EXAMPLE 306

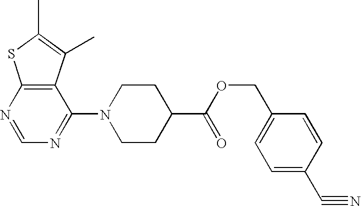

### 4-cyanobenzyl 1-(5,6-dimethylthieno[2,3-d]pyrimidin-4-yl)piperidine-4-carboxylate

### EXAMPLE 307

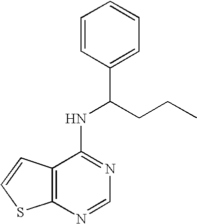

### N-(1-phenylbutyl)thieno[2,3-d]pyrimidin-4-amine

### EXAMPLE 308

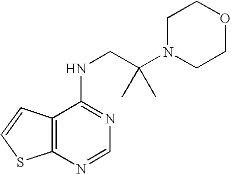

### N-(2-methyl-2-morpholinopropyl)thieno[2,3-d]pyrimidin-4-amine

### EXAMPLE 309

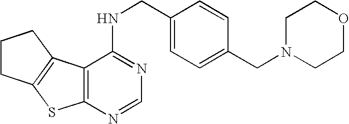

### EXAMPLE 310

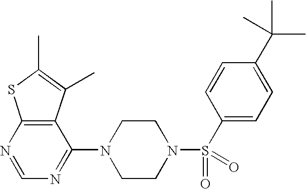

### 4-(4-(4-tert-butylphenylsulfonyl)piperazin-1-yl)-5,6-dimethylthieno[2,3-d]pyrimidine

### EXAMPLE 311

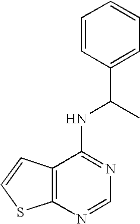

### N-(1-phenylethyl)thieno[2,3-d]pyrimidin-4-amine

### EXAMPLE 312

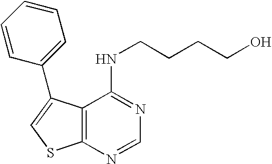

### 4-(5-phenylthieno[2,3-d]pyrimidin-4-ylamino)butan-1-ol

### EXAMPLE 313

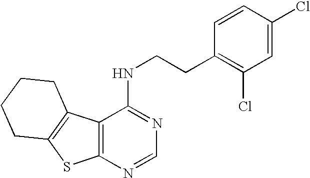

### EXAMPLE 314

This example has intentionally been left blank

### EXAMPLE 315

This example has intentionally been left blank

### EXAMPLE 316

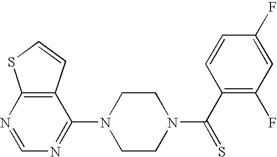

### (2,4-difluorophenyl)(4-(thieno[2,3-d]pyrimidin-4-yl)piperazin-1-yl)methanethione

### EXAMPLE 317

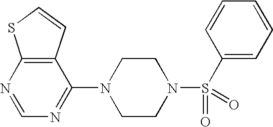

### 5-methyl-4-(4-(phenylsulfonyl)piperazin-1-yl)thieno[2,3-d]pyrimidine

### EXAMPLE 318

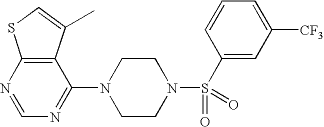

### 4-(4-(3-(trifluoromethyl)phenylsulfonyl)piperazin-1-yl)thieno[2,3-d]pyrimidine

The following compounds can generally be made using the methods known in the art and/or as shown above. It is expected that these compounds when made will have activity similar to those that have been made in the examples above.

The following compounds are represented herein using the Simplified Molecular Input Line Entry System, or SMILES. SMILES is a modern chemical notation system, developed by David Weininger and Daylight Chemical Information Systems, Inc., that is built into all major commercial chemical structure drawing software packages. Software is not needed to interpret SMILES text strings, and an explanation of how to translate SMILES into structures can be found in Weininger, D., J. Chem. Inf. Comput. Sci. 1988, 28, 31-36. All SMILES strings used herein, as well as numerous IUPAC names, were generated using CambridgeSoft's ChemDraw ChemBioDraw Ultra 11.0.
CC1=CSC2=NC═NC(NC3CC(C═CC(C1)=C4)=C4C3)=C21 C1C1=CC(C2)=C(C═C1)CC2NC3=C(C═CC═C4)C4=NC═N3 C1C1=CC(C2)=C(C═C1)CC2NC3=C(C(C)═CC═C4)C4=NC═N3 C1C1=CC(C2)=C(C═C1)CC2NC3=C(C═C(C)C═C4)C4=NC═N3 C1C1=CC(C2)=C(C═C1)CC2NC3=C(C═CC(C)═C4)C4=NC═N3 CC1=CSC2=NC═NC(NC3CC(C═CC(Br)═C4)=C4C3)=C21 BrC1=CC(C2)=C(C═C1)CC2NC3=C(C═CC═C4)C4=NC═N3 BrC1=CC(C2)=C(C═C1)CC2NC3=C(C(C)═CC═C4)C4=NC═N3 BrC1=CC(C2)=C(C═C1)CC2NC3=C(C═C(C)C═C4)C4=NC═N3 BrC1=CC(C2)=C(C═C1)CC2NC3=C(C═CC(C)═C4)C4=NC═N3 CC1=CSC2=NC═NC(NC3CC(C═CC(OC)═C4)=C4C3)=C21 COC1=CC(C2)=C(C═C1)CC2NC3=C(C═CC═C4)C4=NC═N3 CC1=CC═CC2=NC═NC(NC3CC(C═CC(OC)═C4)=C4C3)=C21 CC1=CC2=C(NC3CC(C═CC(OC)═C4)=C4C3)N=CN=C2C═C1 COC1=CC(C2)=C(C═C1)CC2NC3=C(C═CC(C)═C4)C4=NC═N3 CC1=CSC2=NC═NC(NC3CC(C═CC(OCCN(C)C)═C4)=C4C3)=C21 CN(C)CCOC1=CC(C2)=C(C═C1)CC2NC3=C(C═CC═C4)C4=NC═N3 CC1=CC═CC2=NC═NC(NC3CC(C═CC(OCCN(C)C)═C4)=C4C3)=C21 CC1=CC2=C(NC3CC(C═CC(OCCN(C)C)═C4)=C4C3)N=CN=C2C═C1 CC1=CSC2=NC═NC(NC3CC(C═CC(OCCN4CCOCC4)=C5)=C5C3)=C21 C1(NC2CC(C═CC(OCCN3CCOCC3)=C4)=C4C2)=C(C═CC═C5)C5=NC═N1 CC1=CC═CC2=NC═NC(NC3CC(C═CC(OCCN4CCOCC4)=C5)=C5C3)=C21 CC1=CC2=C(NC3CC(C═CC(OCCN4CCOCC4)=C5)=C5C3)N=CN=C2C═C1 C1C1=CC(C2)=C(C═C1)CC2NC3=C(C═C(CN(C)C)S4)C4=NC═N3 C1C1=CC(C2)=C(C═C1)CC2NC3=C(C(CN(C)C)═CS4)C4=NC═N3 C1C1=CC(C2)=C(C═C1)CC2NC3=C(C═CC(CN(C)C)═C4)C4=NC═N3 C1C1=CC(C2)=C(C═C1)CC2NC3=C(C(CN(C)C)═CC═C4)C4=NC═N3 C1C1=CC(C2)=C(C═C1)CC2NC3=C(C═C(CN(C)C)C═C4)C4=NC═N3 C1C1=CC(C2)=C(C═C1)CC2NC3=C(C═C(CN4CCOCC4)S5)C5=NC═N3 C1C1=CC(C2)=C(C═C1)CC2NC3=C(C(CN4CCOCC4)=CS5)C5=NC═N3.C C1C1=CC(C2)=C(C═C1)CC2NC3=C(C═CC(CN4CCOCC4)=C5)C5=NC═N3 C1C1=CC(C2)=C(C═C1)CC2NC3=C(C(CN4CCOCC4)=CC═C5)C5=NC═N3 C1C1=CC(C2)=C(C═C1)CC2NC3=C(C═C(CN4CCOCC4)C═C5)C5=NC═N3 CC1=CSC2=NC═NC(NC3CN(CC4=CC═C(C1)C═C4)CC3)=C21 CC1=CSC2=NC═NC(NC3CN(CC4=CC═CC(C1)=C4)CC3)=C21 CC1=CSC2=NC═NC(NC3CN(CC4=CC═C(OC)C═C4)CC3)=C21 CC1=CSC2=NC═NC(NC3CN(CC4=CC═CC(OC)═C4)CC3)=C21 CC1=CSC2=NC═NC(NC3CCN(CC4=CC═CC(C1)=C4)CC3)=C21 CC1=CSC2=NC═NC(NC3CCN(CC4=CC═C(C1)C═C4)CC3)=C21 CC1=CSC2=NC═NC(NC3CCN(CC4=CC═C(OC)C═C4)CC3)=C21 CC1=CSC2=NC═NC(NC3CCN(CC4=CC═CC(OC)═C4)CC3)=C21 CC1=CSC2=NC═NC(NC3CCN(CC4=CC═C(OCCN(C)C)C═C4)CC3)=C21 CC1=CSC2=NC═NC(NC3CN(CC4=CC═C(OCCN(C)C)C═C4)CC3)=C21 CC1=CSC2=NC═NC(NC3CN(CC4=CC═CC(OCCN(C)C)═C4)CC3)=C21 CC1=CSC2=NC═NC(NC3CCN(CC4=CC═CC(OCCN(C)C)═C4)CC3)=C21 CC1=CSC2=NC═NC(N3CC(N(C(OC(C)C)═O)C)CC3)=C21 CC1=CSC2=NC═NC(N3CC(N(C(OC4CCNCC4)=O)C)CC3)=C21 CC1=CSC2=NC═NC(N3CC(N(C(CC(N)(C)C)═O)C)CC3)=C21 CC1=CSC2=NC═NC(N3CC(N(C(OC(C)(C)C)═O)CCN(C)C)CC3)=C21 CN(C(OC(C)C)═O)C(CC1)CN1C2=C(C═CC═C3)C3=NC═N2 CN(C(OC1CCNCC1)═O)C(CC2)CN2C3=C(C═CC═C4)C4=NC═N3 CN(C(CC(N)(C)C)═O)C(CC1)CN1C2=C(C═CC═C3)C3=NC═N2 O═C(OC(C)(C)C)N(CCN(C)C)C(CC1)CN1C2=C(C═CC═C3)C3=NC═N2 CCC(CC1)CCC1NC2=C(C═CC═C3)C3=NC═N2 COC(CC1)CCC1NC2=C(C═CC═C3)C3=NC═N2 CN(C)CCOC(CC1)CCC1NC2=C(C═CC═C3)C3=NC═N2 C1(NC2CCC(OCCN3CCOCC3)CC2)=C(C═CC═C4)C4=NC═N1 CCC(CC1)CCC1NC2=C(C(C)═CS3)C3=NC═N2 COC(CC1)CCC1NC2=C(C(C)═CS3)C3=NC═N2 CN(C)CCOC(CC1)CCC1NC2=C(C(C)═CS3)C3=NC═N2 CC1=CSC2=NC═NC(NC3CCC(OCCN4CCOCC4)CC3)=C21 C1C(C═C1)=CC═C1CCNC2=C(C═CC═C3)C3=NC(N)=N2 C1C(C═C1)=CC═C1CCNC2=C(C═CC═C3)C3=NC(NC4CCCC4)=N2 C1C(C═C1)=CC═C1CCNC2=C(C═CC═C3)C3=NC(NC4CCNC4)=N2 C1C(C═C1)=CC═C1CCNC2=C(C═CC═C3)C3=NC(NC4CCCCC4)=N2 C1C(C═C1)=CC═C1CCNC2=C(C═CC═C3)C3=NC(NC4CCNCC4)=N2 CC1=CSC2=NC═NC(NC3CCC(CCCN)CC3)=C21 CC1=CSC2=NC═NC(NC3CCC(CCN)CC3)=C21 NCC(CC1)CCC1NC2=C(C(C)═CS3)C3=NC═N2 NCCCC(CC1)CCC1NC2=C(C═CC═C3)C3=NC═N2 NCCC(CC1)CCC1NC2=C(C═CC═C3)C3=NC═N2 NCC(CC1)CCC1NC2=C(C═CC═C3)C3=NC═N2

The activity of the compounds in Examples 1-318 as H₁R and/or H₄R inhibitors is illustrated in the following assay. The other compounds listed above, which have not yet been made and/or tested, are predicted to have activity in these assays as well.

### Biological Activity Assay

In vitro Histamine Receptor Cell-Based Assays

The cell-based assays utilize an aequorin dependent bioluminescence signal. Doubly-transfected, stable CHO—K1 cell lines expressing human H₁ or H₄, mitochondrion-targeted aequorin, and (H₄ only) human G protein Gα16 are obtained from Perkin-Elmer. Cells are maintained in F12 (Ham's) growth medium, containing 10% (vol./vol.) fetal bovine serum, penicillin (100 IU/mL), streptomycin (0.1 mg/mL), zeocin (0.25 mg/mL) and geneticin (0.40 mg/mL). Cell media components are from Invitrogen, Inc. One day prior to assay, the growth medium is replaced with the same, excluding zeocin and geneticin.

For assay preparation, growth medium is aspirated, and cells are rinsed with calcium-free, magnesium-free phosphate-buffered saline, followed by two to three minute incubation in Versene (Invitrogen, Inc.) at 37° C. Assay medium (DMEM:F12 [50:50], phenol-red free, containing 1 mg/mL protease-free bovine serum albumin) is added to collect the released cells, which are then centrifuged. The cell pellet is re-suspended in assay medium, centrifuged once more, and re-suspended in assay medium to a final density of 5×10⁶ cells/mL. Coelenterazine-h dye (500 μM in ethanol) is added to a final concentration of 5 μM, and mixed immediately. The conical tube containing the cells is then wrapped with foil to protect the light-sensitive dye. The cells are incubated for four hours further at room temperature (approximately 21° C.) with end-over-end rotation to keep them in suspension.

Just before assay, the dye-loaded cells are diluted to 0.75×10⁶ cells/mL (H₁ receptor) or 1.5×10⁶ cells/mL (H₄ receptor) with additional assay medium. Cells are dispensed to 1536 well micro-titer plates at 3 μL/well. To assay receptor antagonism, 60 nl of 100× concentration test compounds in 100% dimethyl sulfoxide (DMSO) are dispensed to the wells, one compound per well, by passive pin transfer, and the plates are incubated for 15 minutes at room temperature. Assay plates are then transferred to a Lumilux bioluminescence plate reader (Perkin-Elmer) equipped with an automated 1536 disposable tip pipette. The pipette dispenses 3 μL/well of agonist (histamine, at twice the final concentration, where final concentration is a previously determined EC₈₀) in assay medium, with concurrent bioluminescence detection. Agonist activity of test compounds is excluded by separate assays that measure response to test compounds immediately, without added histamine agonist.

CCD image capture on the Lumilux includes a 5 second baseline read prior to agonist addition, and generally a 40 second read per plate after agonist addition. A decrease in bioluminescence signal (measured either as area-under-the-curve, or maximum signal amplitude minus minimum signal amplitude) correlates with receptor antagonism in a dose dependent manner. The negative control is DMSO lacking any test compound. For antagonist assays, the positive controls are diphenhydramine (2-Diphenylmethoxy-N,N-dimethylethylamine, 10 μM final concentration, H₁ receptor) or JNJ7777120 (1-[(5-Chloro-1H-indol-2-yl)carbonyl]-4-methyl-piperazine, 10 μM final concentration, H₄ receptor). Efficacy is measured as a percentage of positive control activity.

Data reported as NT refers to the example having been not tested. It is expected that these compounds when tested will be active and will have utility similar to those that have been tested.

TABLE 1
Biological Activity

| | | H4 Antagonist EC50, | H1 Antagonist EC50, |
|---|---|---|---|
| | | “+” indicates ≦10 mM, | “+” indicates ≦10 mM, |
| Ex. | SMILES | “−” indicates >10 mM | “−” indicates >10 mM |
| 1 | Cc1csc2ncnc(N3CCN(CC3)c4ccc(Cl)c(Cl)c4)c12 | + | + |
| 2 | Cc1csc2ncnc(N3CCN(CC3)c4ccc(cn4)C(F)(F)F)c12 | − | − |
| 3 | Cc1csc2ncnc(N3CCN(CC3)c4ccccc4Cl)c12 | − | − |
| 4 | COc1ccc(cc1OC)N2CCN(CC2)c3ncnc4scc(C)c34 | − | − |
| 5 | Cc1csc2ncnc(N3CCCC(C3)c4ccc(cc4)C(F)(F)F)c12 | + | + |
| 6 | Cc1csc2ncnc(N3CCN(CC3)c4ccc(Cl)cc4)c12 | + | − |
| 7 | Cc1sc2ncnc(N3CCN(CC3)Cc4ccc5OCOc5c4)c2c1C | + | + |
| 8 | CN1CCC(CC1)N(C)c2ncnc3scc(-c4ccc(Cl)cc4)c23 | + | + |
| 9 | Cc1csc2ncnc(NC3CCN(CC3)Cc4ccccc4)c12 | + | + |
| 10 | Cc1sc2ncnc(N3CCN(CC3)Cc4ccccc4)c2c1C | + | + |
| 11 | C1CN(CCC1Nc2ncnc3sccc23)Cc4ccccc4 | + | + |
| 12 | C1CCc2c(C1)sc3ncnc(N4CCN(CC4)Cc5ccccc5)c23 | − | + |
| 13 | CN1CCC(CC1)N(C)c2ncnc3scc(-c4ccc(Br)cc4)c23 | − | + |
| 14 | CCc1cc2c(ncnc2s1)N3CCN(CC3)Cc4ccccc4 | + | − |
| 15 | This example has | | |
| | intentionally been left | | |
| | blank | | |
| 16 | COc1cccc(CCNc2ncnc3scc(C)c23)c1 | + | − |
| 17 | Cc1csc2ncnc(NCCc3ccc(F)cc3)c12 | + | − |
| 18 | Cc1csc2ncnc(NCCc3cccc(F)c3)c12 | + | − |
| 19 | Cc1ccc(CCNc2ncnc3scc(C)c23)cc1 | + | − |
| 20 | Cc1cccc(CCNc2ncnc3scc(C)c23)c1 | + | − |
| 21 | CC1CCC(CC1)Nc2ncnc3scc(C)c23 | + | − |
| 22 | CCC1CCC(CC1)Nc1ncnc2scc(C)c12 | + | − |
| 23 | Cc1csc2ncnc(CCCc3ccc(Cl)cc3)c12 | + | − |
| 24 | Cc1csc2ncnc(CCC(═O)c3ccc(Cl)cc3)c12 | + | − |
| 25 | Cc1csc2ncnc(C(═O)CCc3ccc(Cl)cc3)c12 | + | − |
| 26 | Cc1csc2ncnc(OCCc3ccc(Cl)cc3)c12 | + | − |
| 27 | Clc1ccc(CCNc2nccc3sccc23)cc1 | − | − |
| 28 | Clc1ccc(CCNc2ncnc3[nH]ccc23)cc1 | − | − |
| 29 | Cc1csc2ncnc(NC3Cc4ccccc4C3)c12 | + | − |
| 30 | Cc1csc2ncnc(NCC3CCc4cc(Cl)ccc34)c12 | + | − |
| 31 | Cc1csc2ncnc(NC(═O)Cc3ccc(Cl)cc3)c12 | + | − |
| 32 | Cc1csc2ncnc(CCNc3ccc(Cl)cc3)c12 | − | − |
| 33 | Cc1csc2ncnc(CC(═O)Nc3ccc(Cl)cc3)c12 | − | − |
| 34 | Cc1csc2ncnc(C(═O)NCc3ccc(Cl)cc3)c12 | − | − |
| 35 | Cc1csc2ncnc(CNCc3ccc(Cl)cc3)c12 | − | − |
| 36 | Cc1csc2nccc(NCCc3ccc(Cl)cc3)c12 | + | − |
| 37 | COc1ccc(CCNc2ccnc3scc(C)c23)cc1 | + | − |
| 38 | Clc1ccc(CCNc2ccnc3sccc23)cc1 | + | − |
| 39 | Cc1csc2ncnc(NCCc3cccc(OCCN4CCOCC4)c3)c12 | NT | NT |
| 40 | CN(C)CCOc1cccc(CCNc2ncnc3scc(C)c23)c1 | NT | NT |
| 41 | CC1CCCC(C1)Nc1ncnc2scc(C)c12 | + | − |
| 42 | Fc1ccccc1CCNc2ncnc3sc4CCCCc4c23 | + | − |
| 43 | Cc1sc2ncnc(NCCc3ccccc3Cl)c2c1C | + | − |
| 44 | CCOC(═O)C1CCN(CC1)c2ncnc3scc(C)c23 | + | − |
| 45 | Clc1ccc(CCNc2ncnc3sccc23)cc1 | + | − |
| 46 | Cc1csc2ncnc(NCCc3ccc(Cl)cc3)c12 | + | − |
| 47 | CCCCNc1ncnc2scc(-c3ccccc3)c12 | + | − |
| 48 | COc1ccc(CCNc2ncnc3sccc23)cc1 | + | − |
| 49 | CNc1ncnc2scc(C)c12 | − | − |
| 50 | CCNc1ncnc2scc(C)c12 | − | − |
| 51 | Cc1csc2ncnc(NCC#C)c12 | − | − |
| 52 | Cc1csc2ncnc(NCC#N)c12 | − | − |
| 53 | Cc1csc2ncnc(NC3CC3)c12 | − | − |
| 54 | CCCNc1ncnc2scc(C)c12 | − | − |
| 55 | Cc1csc2ncnc(NCCN)c12 | − | − |
| 56 | Cc1csc2ncnc(NCCO)c12 | − | − |
| 57 | Cc1csc2ncnc(NCCC#N)c12 | − | − |
| 58 | Cc1csc2ncnc(NC3CCC3)c12 | + | − |
| 59 | Cc1csc2ncnc(NCC3CC3)c12 | + | − |
| 60 | CCC(C)Nc1ncnc2scc(C)c12 | − | − |
| 61 | CC(C)CNc1ncnc2scc(C)c12 | − | − |
| 62 | Cc1csc2ncnc(NCCCO)c12 | − | − |
| 63 | CC(CO)Nc1ncnc2scc(C)c12 | − | − |
| 64 | COCCNc1ncnc2scc(C)c12 | − | − |
| 65 | Cc1csc2ncnc(NC3CCCC3)c12 | + | − |
| 66 | CCCC(C)Nc1ncnc2scc(C)c12 | + | − |
| 67 | CCC(C)CNc1ncnc2scc(C)c12 | + | − |
| 68 | CC(C)C(C)Nc1ncnc2scc(C)c12 | + | − |
| 69 | CC(C)CCNc1ncnc2scc(C)c12 | + | − |
| 70 | CN(C)CCNc1ncnc2scc(C)c12 | − | − |
| 71 | COCC(C)Nc1ncnc2scc(C)c12 | + | − |
| 72 | CCC(CO)Nc1ncnc2scc(C)c12 | + | − |
| 73 | CCC(CO)Nc1ncnc2scc(C)c12 | + | − |
| 74 | Cc1csc2ncnc(NCCCCO)c12 | − | − |
| 75 | Cc1csc2ncnc(NCc3ccco3)c12 | + | − |
| 76 | Cc1csc2ncnc(NC3CCCCC3)c12 | + | − |
| 77 | Cc1csc2ncnc(NCC3CCCO3)c12 | + | − |
| 78 | Cc1csc2ncnc(NCCC(C)(C)C)c12 | + | − |
| 79 | CC(C)CC(C)Nc1ncnc2scc(C)c12 | + | − |
| 80 | Cc1csc2ncnc(NC3CONC3═O)c12 | − | − |
| 81 | Cc1csc2ncnc(NN3CCOCC3)c12 | − | − |
| 82 | CC(C)C(CO)Nc1ncnc2scc(C)c12 | + | − |
| 83 | Cc1csc2ncnc(NCc3ccccc3)c12 | + | − |
| 84 | Cc1csc2ncnc(NCc3cccnc3)c12 | − | − |
| 85 | Cc1csc2ncnc(NCc3ccccn3)c12 | − | − |
| 86 | Cc1csc2ncnc(NCc3ccncc3)c12 | − | − |
| 87 | Cc1csc2ncnc(NCc3cccs3)c12 | + | − |
| 88 | This example has | | |
| | intentionally been left | | |
| | blank | | |
| 89 | Cc1csc2ncnc(NCC3CCCCC3)c12 | + | − |
| 90 | Cc1csc2ncnc(NC3CCCCCC3)c12 | + | − |
| 91 | CC1CCCCC1Nc2ncnc3scc(C)c23 | + | − |
| 92 | Cc1csc2ncnc(NCCN3CCCC3)c12 | − | − |
| 93 | CN1CCN(CC1)Nc2ncnc3scc(C)c23 | − | − |
| 94 | CCN(CC)CCNc1ncnc2scc(C)c12 | − | − |
| 95 | Cc1cccc(CNc2ncnc3scc(C)c23)c1 | + | − |
| 96 | C[C@H](Nc1ncnc2scc(C)c12)c3ccccc3 | + | − |
| 97 | C[C@@H](Nc1ncnc2scc(C)c12)c3ccccc3 | + | − |
| 98 | Cc1csc2ncnc(NCCc3ccccc3)c12 | + | + |
| 99 | Cc1ccccc1CNc2ncnc3scc(C)c23 | + | − |
| 100 | Cc1ccc(CNc2ncnc3scc(C)c23)cc1 | + | − |
| 101 | Cc1cnc(CNc2ncnc3scc(C)c23)cn1 | − | − |
| 102 | Cc1csc2ncnc(NCc3cccc(F)c3)c12 | + | − |
| 103 | Cc1csc2ncnc(NCc3ccc(F)cc3)c12 | + | − |
| 104 | Cc1csc2ncnc(NCc3ccccc3F)c12 | + | − |
| 105 | Cc1csc2ncnc(NCCCn3ccnc3)c12 | − | − |
| 106 | CN1CCCC1CCNc2ncnc3scc(C)c23 | − | − |
| 107 | Cc1csc2ncnc(NCCN3CCCCC3)c12 | − | − |
| 108 | CC(C)CCCC(C)Nc1ncnc2scc(C)c12 | + | − |
| 109 | Cc1csc2ncnc(NCCN3CCOCC3)c12 | − | − |
| 110 | CCOC(═O)CC(C)Nc1ncnc2scc(C)c12 | + | − |
| 111 | Cc1csc2ncnc(NC3CCc4ccccc34)c12 | + | − |
| 112 | This example has | | |
| | intentionally been left | | |
| | blank | | |
| 113 | Cc1ccc(C)c(CNc2ncnc3scc(C)c23)c1 | + | − |
| 114 | C[C@H](Nc1ncnc2scc(C)c12)c3ccc(C)cc3 | + | − |
| 115 | C[C@@H](Nc1ncnc2scc(C)c12)c3ccc(C)cc3 | − | − |
| 116 | CC(CNc1ncnc2scc(C)c12)c3ccccc3 | + | − |
| 117 | Cc1csc2ncnc(NCCCc3ccccc3)c12 | + | − |
| 118 | Cc1ccc(CNc2ncnc3scc(C)c23)cc1C | + | − |
| 119 | CC(CNc1ncnc2scc(C)c12)c3ccccc3 | + | − |
| 120 | CC(Cc1ccncc1)Nc2ncnc3scc(C)c23 | − | − |
| 121 | Cc1csc2ncnc(NCCc3ccc(O)cc3)c12 | + | − |
| 122 | Cc1csc2ncnc(NCCOc3ccccc3)c12 | + | − |
| 123 | COc1ccccc1CNc2ncnc3scc(C)c23 | + | − |
| 124 | COc1ccc(CNc2ncnc3scc(C)c23)cc1 | + | − |
| 125 | COc1cccc(CNc2ncnc3scc(C)c23)c1 | + | − |
| 126 | This example has | | |
| | intentionally been left | | |
| | blank | | |
| 127 | Cc1csc2ncnc(NCCc3ccccc3F)c12 | + | − |
| 128 | This example has | | |
| | intentionally been left | | |
| | blank | | |
| 129 | Cc1csc2ncnc(NCc3cccc(Cl)c3)c12 | + | − |
| 130 | Cc1csc2ncnc(NCc3ccccc3Cl)c12 | + | − |
| 131 | Cc1csc2ncnc(NCc3ccc(Cl)cc3)c12 | + | − |
| 132 | Cc1csc2ncnc(NCCCN3CCCC3═O)c12 | − | − |
| 133 | Cc1csc2ncnc(NCc3ccc(F)cc3F)c12 | + | − |
| 134 | Cc1csc2ncnc(NCc3ccc(F)c(F)c3)c12 | + | − |
| 135 | Cc1csc2ncnc(NCc3cc(F)cc(F)c3)c12 | + | − |
| 136 | Cc1csc2ncnc(NCCCN3CCOCC3)c12 | + | − |
| 137 | CC(C)N(CCNc1ncnc2scc(C)c12)C(C)C | − | − |
| 138 | Cc1csc2ncnc(NC3CCCc4ccccc34)c12 | − | − |
| 139 | CC(CCc1ccccc1)Nc2ncnc3scc(C)c23 | + | − |
| 140 | CC(C)c1ccc(CNc2ncnc3scc(C)c23)cc1 | + | − |
| 141 | Cc1csc2ncnc(NCc3ccc4OCOc4c3)c12 | + | − |
| 142 | This example has | | |
| | intentionally been left | | |
| | blank | | |
| 143 | Cc1csc2ncnc(N[C@H](CO)Cc3ccccc3)c12 | + | − |
| 144 | COc1ccc(CCNc2ncnc3scc(C)c23)cc1 | + | + |
| 145 | COc1ccccc1CCNc2ncnc3scc(C)c23 | + | − |
| 146 | CCOc1ccccc1CNc2ncnc3scc(C)c23 | + | − |
| 147 | COc1ccc(Nc2ncnc3scc(C)c23)cc1OC | − | − |
| 148 | Cc1csc2ncnc(NCCc3ccccc3Cl)c12 | + | − |
| 149 | Cc1csc2ncnc(NCCc3cccc(Cl)c3)c12 | + | − |
| 150 | Cc1csc2ncnc(NC3CC(C)(C)NC(C)(C)C3)c12 | − | − |
| 151 | CCN(CC)CCCC(C)Nc1ncnc2scc(C)c12 | − | − |
| 152 | Cc1csc2ncnc(NCc3ccc(F)c(Cl)c3)c12 | + | − |
| 153 | Cc1csc2ncnc(N(CCC#N)Cc3ccccc3)c12 | − | − |
| 154 | Cc1csc2ncnc(NCCNC(═O)OC(C)(C)C)c12 | + | − |
| 155 | Cc1csc2ncnc(NCCc3c[nH]c4ccccc34)c12 | + | − |
| 156 | Cc1csc2ncnc(NCc3ccc(cc3)C(C)(C)C)c12 | + | − |
| 157 | CN(CCCNc1ncnc2scc(C)c12)c3ccccc3 | + | − |
| 158 | Cc1csc2ncnc(NCC3(O)CCCCC3)c12 | − | − |
| 159 | COc1cc(CNc2ncnc3scc(C)c23)cc(OC)c1 | + | − |
| 160 | COc1ccc(CNc2ncnc3scc(C)c23)cc1OC | + | − |
| 161 | Cc1csc2ncnc(NCC(═O)c3ccccc3)c12 | − | − |
| 162 | CCOC(═O)N1CCC(CC1)Nc2ncnc3scc(C)c23 | + | − |
| 163 | Cc1csc2ncnc(NCCCNC(═O)OC(C)(C)C)c12 | + | − |
| 164 | Cc1csc2ncnc(NCc3ccc(cc3)C(F)(F)F)c12 | + | − |
| 165 | Cc1csc2ncnc(NCc3cccc(c3)C(F)(F)F)c12 | + | − |
| 166 | Cc1csc2ncnc(NCc3ccc(Cl)c(Cl)c3)c12 | + | − |
| 167 | Cc1csc2ncnc(NCc3ccc(Cl)cc3Cl)c12 | + | − |
| 168 | Cc1csc2ncnc(NC3CCN(C3)Cc4ccccc4)c12 | + | − |
| 169 | COc1ccc(OC)c(CCNc2ncnc3scc(C)c23)c1 | + | − |
| 170 | CN(C)c1ccc(CNc2ncnc3scc(C)c23)cc1 | − | − |
| 171 | COc1ccc(CNc2ncnc3scc(C)c23)cc1O | − | − |
| 172 | Cc1csc2ncnc(NCc3ccccc3OC(F)(F)F)c12 | − | − |
| 173 | Cc1csc2ncnc(NCCc3ccc(cc3)S(N)(═O)═O)c12 | + | − |
| 174 | COc1ccc(cc1)C(═O)CNc2ncnc3scc(C)c23 | − | − |
| 175 | Cc1csc2ncnc(NCCc3ccc4OCOc4c3)c12 | + | − |
| 176 | Cc1csc2ncnc(NCCC(c3ccccc3)c4ccccc4)c12 | + | − |
| 177 | Cc1ccc(cc1)S(═O)(═O)NCCNc2ncnc3scc(C)c23 | − | − |
| 178 | CN(C)c1ncnc2scc(C)c12 | − | − |
| 179 | CCN(C)c1ncnc2scc(C)c12 | − | − |
| 180 | CN(CC#C)c1ncnc2scc(C)c12 | − | − |
| 181 | CCN(CC)c1ncnc2scc(C)c12 | − | − |
| 182 | CCCN(C)c1ncnc2scc(C)c12 | − | − |
| 183 | CN(CCO)c1ncnc2scc(C)c12 | − | − |
| 184 | CN(CCC#N)c1ncnc2scc(C)c12 | − | − |
| 185 | CC(C)CN(C)c1ncnc2scc(C)c12 | − | − |
| 186 | CCN(C(C)C)c1ncnc2scc(C)c12 | − | − |
| 187 | CCCCN(C)c1ncnc2scc(C)c12 | + | − |
| 188 | CCN(CCO)c1ncnc2scc(C)c12 | − | − |
| 189 | Cc1csc2ncnc(N3CCSC3)c12 | + | − |
| 190 | CC1CCCCN1c2ncnc3scc(C)c23 | + | − |
| 191 | CC1CCN(CC1)c2ncnc3scc(C)c23 | − | − |
| 192 | CN1CCN(CC1)c2ncnc3scc(C)c23 | + | − |
| 193 | Cc1csc2ncnc(N3CCCC3CO)c12 | − | − |
| 194 | Cc1csc2ncnc(N3CCC[C@@H]3CO)c12 | − | − |
| 195 | CCCCN(CC)c1ncnc2scc(C)c12 | − | − |
| 196 | CCCN(CCC)c1ncnc2scc(C)c12 | − | − |
| 197 | Cc1csc2ncnc(N3CCSCC3)c12 | − | − |
| 198 | Cc1csc2ncnc(N(CCO)CCO)c12 | − | − |
| 199 | CC1CC(C)CN(C1)c2ncnc3scc(C)c23 | − | − |
| 200 | CN(C1CCCCC1)c2ncnc3scc(C)c23 | − | − |
| 201 | CC1CCCC(C)N1c2ncnc3scc(C)c23 | − | − |
| 202 | CN(C)C1CCN(C1)c2ncnc3scc(C)c23 | − | + |
| 203 | C[C@@H]1CN(C[C@H](C)N1)c2ncnc3scc(C)c23 | − | − |
| 204 | Cc1csc2ncnc(N3CCCC(CO)C3)c12 | − | − |
| 205 | COC[C@@H]1CCCN1c2ncnc3scc(C)c23 | + | − |
| 206 | Cc1csc2ncnc(N3CCCCC3CO)c12 | + | − |
| 207 | CCN(CCCCO)c1ncnc2scc(C)c12 | − | − |
| 208 | Cc1csc2ncnc(N3Cc4ccccc4C3)c12 | − | − |
| 209 | CN(Cc1ccccc1)c2ncnc3scc(C)c23 | − | − |
| 210 | CCN(C1CCCCC1)c2ncnc3scc(C)c23 | − | − |
| 211 | Cc1csc2ncnc(N3CCC(CC3)C(N)═O)c12 | − | − |
| 212 | CC(═O)N[C@H]1CCN(C1)c2ncnc3scc(C)c23 | − | − |
| 213 | Cc1csc2ncnc(N3CCCC(C3)C(N)═O)c12 | − | − |
| 214 | CC(═O)N[C@H]1CCN(C1)c2ncnc3scc(C)c23 | − | − |
| 215 | Cc1csc2ncnc(N3CCCCC3CCO)c12 | + | − |
| 216 | Cc1csc2ncnc(N3CCc4ccccc4C3)c12 | + | − |
| 217 | CCN(Cc1ccccc1)c2ncnc3scc(C)c23 | + | − |
| 218 | CN(CCc1ccccc1)c2ncnc3scc(C)c23 | + | + |
| 219 | CCN(Cc1ccccc1)c2ncnc3scc(C)c23 | + | − |
| 220 | CN(CCc1ccccn1)c2ncnc3scc(C)c23 | − | + |
| 221 | CN(CCc1ccccn1)c2ncnc3scc(C)c23 | − | + |
| 222 | Cc1csc2ncnc(N3CCCC4CCCCC34)c12 | − | − |
| 223 | Cc1csc2ncnc(N(CC═C)C3CCCCC3)c12 | − | − |
| 224 | COC(═O)C1CCN(CC1)c2ncnc3scc(C)c23 | + | − |
| 225 | CC(C)N(Cc1ccccc1)c2ncnc3scc(C)c23 | − | − |
| 226 | Cc1csc2ncnc(N(CCO)Cc3ccccc3)c12 | + | − |
| 227 | CN(CC(O)c1ccccc1)c2ncnc3scc(C)c23 | − | − |
| 228 | Cc1csc2ncnc(N3CCC(O)(O)CC3)c12 | − | − |
| 229 | CCOC(═O)C1CCCN(C1)c2ncnc3scc(C)c23 | + | + |
| 230 | CCOC(═O)N1CCN(CC1)c2ncnc3scc(C)c23 | − | − |
| 231 | Cc1csc2ncnc(N(CCC#N)Cc3ccccc3)c12 | − | − |
| 232 | Cc1csc2ncnc(N(CCC#N)Cc3cccnc3)c12 | − | − |
| 233 | Cc1csc2ncnc(N3CCN(CC3)c4ccccc4)c12 | − | − |
| 234 | Cc1csc2ncnc(N3CCN(CC3)c4ccccn4)c12 | − | − |
| 235 | CCCCN(Cc1ccccc1)c2ncnc3scc(C)c23 | + | − |
| 236 | C[C@@H](N(CCO)c1ncnc2scc(C)c12)c3ccccc3 | − | − |
| 237 | Cc1csc2ncnc(N(CCCO)Cc3ccccn3)c12 | − | − |
| 238 | CN(CC(O)c1ccc(O)cc1)c2ncnc3scc(C)c23 | − | − |
| 239 | Cc1csc2ncnc(N3CCN(CC3)C4CCCCC4)c12 | − | − |
| 240 | Cc1csc2ncnc(N3CCC(CC3)Cc4ccccc4)c12 | + | − |
| 241 | Cc1csc2ncnc(N3CCN(CC3)Cc4ccccc4)c12 | + | + |
| 242 | Cc1csc2ncnc(N3CCCN(CC3)Cc4ccccc4)c12 | − | − |
| 243 | Cc1csc2ncnc(N3CCN(CC3)c4ccc(O)cc4)c12 | − | − |
| 244 | CN(C)CCN(Cc1ccccc1)c2ncnc3scc(C)c23 | − | − |
| 245 | Cc1csc2ncnc(N3CCN(CC3)c4ccccc4F)c12 | − | − |
| 246 | Cc1csc2ncnc(N3CCN(CC3)c4ccc(F)cc4)c12 | − | − |
| 247 | Cc1csc2ncnc(N3CCN(CC3)CC4CCCCC4)c12 | + | − |
| 248 | CN(C[C@H](O)c1ccc(O)c(O)c1)c2ncnc3scc(C)c23 | − | − |
| 249 | CCOC(═O)CC1N(CCNC1═O)c2ncnc3scc(C)c23 | − | − |
| 250 | Cc1csc2ncnc(N3CCN(CC3)C(═O)OC(C)(C)C)c12 | + | − |
| 251 | Cc1csc2ncnc(N3CCC(C3)NC(═O)OC(C)(C)C)c12 | + | − |
| 252 | Cc1csc2ncnc(N3CCN(CC3)c4ccccc4C#N)c12 | + | − |
| 253 | Cc1csc2ncnc(N3CCN(CC3)c4ccc(cn4)C#N)c12 | − | + |
| 254 | Cc1cccc(N2CCN(CC2)c3ncnc4scc(C)c34)c1C | − | − |
| 255 | CC1CN(CCN1c2cccc(C)c2)c3ncnc4scc(C)c34 | + | − |
| 256 | Cc1csc2ncnc(N3CCN(CC3)CCc4ccccc4)c12 | + | + |
| 257 | Cc1ccc(cc1C)N2CCN(CC2)c3ncnc4scc(C)c34 | − | − |
| 258 | Cc1ccc(N2CCN(CC2)c3ncnc4scc(C)c34)c(C)c1 | + | − |
| 259 | Cc1ccc(C)c(c1)N2CCN(CC2)c3ncnc4scc(C)c34 | − | + |
| 260 | Cc1cccc(N2CCN(CC2)c3ncnc4scc(C)c34)c1C | − | − |
| 261 | COc1ccc(cc1)N2CCN(CC2)c3ncnc4scc(C)c34 | − | − |
| 262 | COc1ccccc1N2CCN(CC2)c3ncnc4scc(C)c34 | − | − |
| 263 | COc1cccc(c1)N2CCN(CC2)c3ncnc4scc(C)c34 | − | − |
| 264 | COc1ccc(CCN(C)c2ncnc3scc(C)c23)cc1OC | − | − |
| 265 | Cc1csc2ncnc(N3CCN(CC3)c4cccc(Cl)c4)c12 | − | − |
| 266 | Cc1csc2ncnc(N3CCN(CC3)c4ccc(Cl)cc4)c12 | − | − |
| 267 | Cc1csc2ncnc(N3CCN(CC3)c4ccc(F)cc4F)c12 | − | − |
| 268 | Cc1csc2ncnc(N(Cc3cccnc3)Cc4cccnc4)c12 | − | − |
| 269 | Cc1csc2ncnc(N3CCN(CCN4CCOCC4)CC3)c12 | + | − |
| 270 | CN(C1CCN(C1)c2ncnc3scc(C)c23)C(═O)OC(C)(C)C | + | − |
| 271 | CC(═O)c1ccc(cc1)N2CCN(CC2)c3ncnc4scc(C)c34 | + | − |
| 272 | CCN(CC)CCN(Cc1ccccc1)c2ncnc3scc(C)c23 | − | − |
| 273 | Cc1csc2ncnc(N3CCN(CC3)c4ccc(cc4)[N+]([O−])═O)c12 | + | − |
| 274 | CN(Cc1cccc2ccccc12)c3ncnc4scc(C)c34 | − | − |
| 275 | Cc1ccc(Cl)cc1N2CCN(CC2)c3ncnc4scc(C)c34 | − | − |
| 276 | COc1cc(CN(C)c2ncnc3scc(C)c23)cc(OC)c1OC | − | − |
| 277 | Cc1csc2ncnc(N(CCc3ccccc3)Cc4ccccc4)c12 | + | − |
| 278 | CN(C(Cc1ccccc1)c2ccccc2)c3ncnc4scc(C)c34 | − | − |
| 279 | Cc1csc2ncnc(N(CCc3ccccc3)Cc4ccccc4)c12 | + | − |
| 280 | Cc1csc2ncnc(N3CCC(O)(CC3)c4ccc(Cl)cc4)c12 | − | − |
| 281 | Cc1csc2ncnc(N(CC#C)Cc3ccc(Cl)cc3Cl)c12 | − | − |
| 282 | CCN(C(Cc1ccccc1)c2ccco2)c3ncnc4scc(C)c34 | − | − |
| 283 | Cc1csc2ncnc(N3CCN(C[C@H]3CO)C(═O)OC(C)(C)C)c12 | − | − |
| 284 | Cc1csc2ncnc(N3CCN(CC3)c4ccc(cc4)C(C)(C)C)c12 | − | − |
| 285 | Cc1csc2ncnc(N3CCN(CC3)Cc4ccc5OCOc5c4)c12 | + | + |
| 286 | COc1cc2CCN(Cc2cc1OC)c3ncnc4scc(C)c34 | − | − |
| 287 | Cc1csc2ncnc(N3CCN(CC3)c4ccc(cc4)C(F)(F)F)c12 | + | − |
| 288 | Cc1csc2ncnc(N3CCN(CC3)c4cccc(c4)C(F)(F)F)c12 | − | − |
| 289 | Clc1ccc(cc1)S(═O)(═O)NNc2ncnc3sccc23 | + | + |
| 290 | C(Cc1ccccc1)Nc2ncnc3ccsc23 | + | − |
| 291 | This example has | | |
| | intentionally been left | | |
| | blank | | |
| 292 | This example has | | |
| | intentionally been left | | |
| | blank | | |
| 293 | C(Cc1ccccc1)Nc2ncnc3scc(-c4ccccc4)c23 | + | − |
| 294 | This example has | | |
| | intentionally been left | | |
| | blank | | |
| 295 | Cc1csc2c(NCCc3ccc(Cl)cc3)ncnc12 | + | − |
| 296 | COc1ccc(CCNc2nc(C)nc3sc(C)c(C)c23)cc1OC | + | − |
| 297 | Clc1ccccc1CCNc2ncnc3sc4CCCCc4c23 | + | − |
| 298 | Cc1sc2ncnc(NCCc3ccccc3)c2c1C | + | − |
| 299 | COc1ccccc1CNc2ncnc3sc(C)c(C)c23 | + | − |
| 300 | CCOC(═O)CNc1ncnc2scc(-c3ccccc3)c12 | + | − |
| 301 | Cc1sc2ncnc(NCCc3ccc(cc3)S(N)(═O)═O)c2c1C | + | − |
| 302 | C1CN(CCC1Cc2ccccc2)c3ncnc4sccc34 | + | − |
| 303 | COCC(C)n1c(C)cc(C(═O)CSc2ncnc3sc(C)c(C)c23)c1C | + | − |
| 304 | CC(═O)c1c(C)[nH]c(C(═O)CSc2ncnc3sc(C)c(C)c23)c1C | + | − |
| 305 | This example has | | |
| | intentionally been left | | |
| | blank | | |
| 306 | Cc1sc2ncnc(N3CCC(CC3)C(═O)OCc4ccc(cc4)C#N)c2c1C | + | − |
| 307 | CCCC(Nc1ncnc2sccc12)c3ccccc3 | + | − |
| 308 | Cc1sc2ncnc(NCC(C)(C)N3CCOCC3)c2c1C | + | − |
| 309 | C1Cc2sc3ncnc(NCc4ccc(CN5CCOCC5)cc4)c3c2C1 | + | − |
| 310 | Cc1sc2ncnc(N3CCN(CC3)S(═O)(═O)c4ccc(cc4)C(C)(C)C)c2c1C | + | − |
| 311 | CC(Nc1ncnc2sccc12)c3ccccc3 | + | − |
| 312 | OCCCCNc1ncnc2scc(-c3ccccc3)c12 | + | − |
| 313 | Clc1ccc(CCNc2ncnc3sc4CCCCc4c23)c(Cl)c1 | + | − |
| 314 | This example has | | |
| | intentionally been left | | |
| | blank | | |
| 315 | This example has | | |
| | intentionally been left | | |
| | blank | | |
| 316 | Fc1ccc(NC(═S)N2CCN(CC2)c3ncnc4sccc34)c(F)c1 | + | − |
| 317 | O═S(═O)(N1CCN(CC1)c2ncnc3sccc23)c4ccccc4 | + | − |
| 318 | Cc1csc2ncnc(N3CCN(CC3)S(═O)(═O)c4cccc(c4)C(F)(F)F)c12 | + | − |

### In Vivo Assay Number Two

### Allergic Conjunctivitis in Passively Sensitized Guinea Pigs

Male Hartley VAF outbred guinea pigs were passively sensitized to ovalbumin by a single OD subconjunctival injection of undiluted guinea pig anti-ovalbumin antiserum 24 hours before OD topical challenge with 500 μg ovalbumin in saline. Control animals were injected with saline only and challenged with ovalbumin. To determine acute phase drug efficacy, 30 min after challenge animals were clinically scored by a masked observer for severity of signs of conjunctivitis based on a standard scale. Test compounds were administered topically 1 hour prior to challenge (QD protocol), or 1 hour prior to challenge and again 8 hours after challenge (BID protocol). Twenty-four hours after challenge, animals were euthanized and conjunctivae were harvested for determination of tissue eosinophil peroxidase (EPO) concentration as a marker of allergic inflammation. Homogenates of freshly collected tissues were prepared by shaking the tissues in 2 mL round-bottom tubes containing 0.5 mL of homogenization buffer (50 mM Tris HCl, pH 8.0, 6 mM KBr) and one 5-mm stainless steel bead on a Qiagen TissueLyser at 30 Hz for 5 min. Homogenates were frozen and thawed once, then centrifuged at 10,000 rpm for 5 min. EPO activity in supernatants was measured by reacting diluted homogenates with a solution of 6 mM o-phenylenediamine substrate and 8.8 mM H2O2 in homogenization buffer for 3 min. The reaction was stopped with 4M H2SO4 and absorbances were measured at 490 nm on a spectrophotometric plate reader. Total EPO in samples was calculated from a standard curve of recombinant human EPO in each assay. EPO activity was normalized to total protein concentration (Pierce BCA assay) in supernatants. Background EPO activity was determined from the unsensitized, antigen-challenged control group. Percent inhibition was calculated from the sensitized, antigen-challenged, vehicle-treated control group in each experiment. Ovalbumin-injected animals dosed topically with 0.1% w/v dexamethasone (dex) served as positive control. Groups were compared by ANOVA with Dunnett's or Tukey's post-hoc tests where appropriate with significance assigned at the 95% confidence level.

The table below summarizes the results. In the column labeled “BID activity”, a test compound was assigned a “+” if a 0.01% bid dose was statistically equivalent to dexamethasone with respect to reduction of EPO activity, while a “−” was assigned if the compound was statistically inferior to dexamethasone and not different than vehicle. In the column labeled “QD activity”, a test compound was assigned a “+” if a ≦0.1% qd dose was statistically equivalent to dexamethasone with respect to reduction of EPO activity, while a “−” was assigned if the compound was statistically inferior to dexamethasone and not different than vehicle.

Data reported as NT refers to the example having been not tested. It is expected that these compounds when tested will be active and will have utility similar to those that have been tested.

TABLE 3
In Vivo Activity

| Example # | BID activity | QD activity |
|---|---|---|
| 43 | − | NT |
| 45 | + | − |
| 29 | NT | + |
| 27 | + | − |
| 85 | NT | + |
| 215 | NT | NT |

### Compositions

The following are examples of compositions which may be used to orally deliver compounds disclosed herein as a capsule.

A solid form of a compound of Formula (I) may be passed through one or more sieve screens to produce a consistent particle size. Excipients, too, may be passed through a sieve. Appropriate weights of compounds, sufficient to achieve the target dosage per capsule, may be measured and added to a mixing container or apparatus, and the blend is then mixed until uniform. Blend uniformity may be done by, for example, sampling 3 points within the container (top, middle, and bottom) and testing each sample for potency. A test result of 95-105% of target, with an RSD of 5%, would be considered ideal; optionally, additional blend time may be allowed to achieve a uniform blend. Upon acceptable blend uniformity results, a measured aliquot of this stock formulation may be separated to manufacture the lower strengths. Magnesium stearate may be passed through a sieve, collected, weighed, added to the blender as a lubricant, and mixed until dispersed. The final blend is weighed and reconciled. Capsules may then be opened and blended materials flood fed into the body of the capsules using a spatula. Capsules in trays may be tamped to settle the blend in each capsule to assure uniform target fill weight, and then sealed by combining the filled bodies with the caps.

### COMPOSITION EXAMPLE 1

10 mg Capsule: Total fill weight of capsule is 300 mg, not including capsule weight. Target compound dosage is 10 mg per capsule, but may be adjusted to account for the weight of counterions and/or solvates if given as a salt or solvated polymorph thereof. In such a case the weight of the other excipients, typically the filler, is reduced.

| Ingredient | Quantity per Capsule, mg |
|---|---|
| Compound of Formula (I) | 10.00 |
| Lactose monohydrate | 269.00 |
| Silicon dioxide | 3.00 |
| Crospovidone | 15.00 |
| Magnesium stearate (vegetable grade) | 3.00 |

### COMPOSITION EXAMPLE 2

20 mg Capsule: Total fill weight of capsule is 300 mg, not including capsule weight. Target compound dosage is 20 mg per capsule, but may be adjusted to account for the weight of counterions and/or solvates if given as a salt or solvated polymorph thereof. In such a case the weight of the other excipients, typically the filler, is reduced.

| Ingredient | Quantity per Capsule, mg |
|---|---|
| Compound of Formula (I) | 20.00 |
| Microcrystalline cellulose (MCC) | 277.00 |
| Magnesium stearate (vegetable grade) | 3.00 |

The following are examples of compositions which may be used to topically deliver compounds disclosed herein, for example to the eye or nasal passages.

### COMPOSITION EXAMPLE 3

| Ingredients | Concentration (w/v %) |
|---|---|
| Compound of Formula (I) | 0.01-2% |
| Hydroxypropyl methylcellulose | 0.5% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### COMPOSITION EXAMPLE 4

| Ingredients | Concentration (w/v %) |
|---|---|
| Compound of Formula (I) | 0.01-2% |
| White petrolatum and mineral oil and lanolin | Ointment consistency |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method of treatment of a H₁R and/or H₄R-mediated disease comprising the administration of a therapeutically effective amount of a compound of structural Formula I: 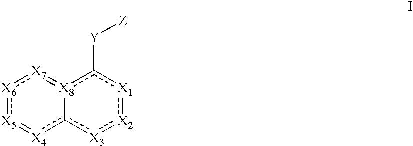
or a salt thereof, wherein:
a dashed line indicates that a bond may be present or absent;
X₁ and X₃ are independently selected from the group consisting of [C(R₂)(R₃)] and NR₄;
X₂ is selected from the group consisting of [C(R₅)(R₆)], NR₇, O, and S;
X₄ is selected from the group consisting of [C(R₈)(R₉)], NR₁₀, O, and S;
X₅ is selected from the group consisting of [C(R₁₁)(R₁₂)], NR₁₃, O, and S;
X₆ is selected from the group consisting of [C(R₁₄)(R₁₅)], NR₁₆, O, and S;
X₇ is selected from the group consisting of [C(R₁₇)(R₁₈)], NR₁₉, O, S, and a bond;
X₈ is selected from the group consisting of C and N;
taken together, X₁ to X₈ form a fully aromatic bicyclic system;
Y is selected from the group consisting of a bond, NR₁[C(R₂₀)(R₂₁)]ₙ, NR₁[C(R₂₂)(R₂₃)]_{n—W—[C(R}₂₄)(R₂₅)]ₘ, S—[C(R₂₆)(R₂₇)]ₙ—W—[C(R₂₈)(R₂₉)]ₘ, O[C(R₃₀)(R₃₁)]ₙ, [C(R₃₂)(R₃₃)]ₙ—W—[C(R₃₄)(R₃₅)]ₘ, and [C(R₃₆)(R₃₇)]ₙ;
n and m are each independently an integer from 0 to 3;
W is selected from the group consisting of O, S, S(O)₂, NR₃₈, NR₃₉S(O₂), C(O), C(S), C(O)O, C(O)NR₄₀, NR₄₁C(O), and NR₄₂C(O)O;
Z is selected from the group consisting of hydrogen, aryl, alkyl, heterocycloalkyl, and cycloalkyl, any of which may be optionally substituted;
R₁ to R₄₂ are each independently selected from the group consisting of null, hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
R₁₁ and R₁₄ may be joined together to form a partially saturated cycloalkyl; and
R₁ and R₂₀, or R₁ and R₂₂, or R₂₂ and R₃₈, or R₁ and R₃₈, may be joined together to form a heterocycloalkyl.

2. The method as recited in claim 1, wherein said compound has structural Formula II: 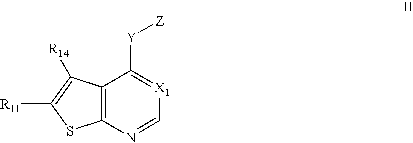
or a salt thereof, wherein:
X₁ is selected from the group consisting of [C(R₂)] and N;
Y is selected from the group consisting of a bond, NR₁[C(R₂₀)(R₂₁)]ₙ, NR₁[C(R₂₂)(R₂₃)]ₙ—W—[C(R₂₄)(R₂₅)]ₘ, S—[C(R₂₆)(R₂₇)]ₙ—W—[C(R₂₈)(R₂₉)]ₘ, O[C(R₃₀)(R₃₁)]ₙ, [C(R₃₂)(R₃₃)]ₙ—W—[C(R₃₄)(R₃₅)]ₘ, and [C(R₃₆)(R₃₇)]ₙ;
n and m are each independently an integer from 0 to 3;
W is selected from the group consisting of O, S, S(O)₂, NR₃₈, NR₃₉S(O₂), C(O), C(S), C(O)O, C(O)NR₄₀, NR₄₁C(O), and NR₄₂C(O)O;
Z is selected from the group consisting of aryl, alkyl, heterocycloalkyl, alkoxylcarbonyl, acyl, and cycloalkyl, any of which may be optionally substituted;
R₁, R₂, R₁₄, and R₂₀ to R₄₂ are each independently selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
R₁₁ is selected from the group consisting of hydrogen, alkyl, heteroalkyl, alkoxy, halogen, haloalkyl, amino, aminoalkyl, amido, carboxyl, acyl, hydroxy, cyano, nitro, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
R₁₁ and R₁₄ may be joined together to form a partially saturated cycloalkyl; and
R₁ and R₂₀, or R₁ and R₂₂, or R₂₂ and R₃₈, or R₁ and R₃₈, may be joined together to form a heterocycloalkyl;
and with the provisos that;
if Y is NR₁[C(R₂₀)(R₂₁)]ₙ, R₁ is hydrogen, and n is 0, then Z is not aryl or heteroaryl; and
if Y is NR₁[C(R₂₂)(R₂₃)]ₙ—W—[C(R₂₄)(R₂₅)]ₘ, n is 2, m is 0, W is NR₃₈, R₂₂, and R₂₃ are hydrogen, and R₁ and R₃₈ are joined together to form a piperazine ring, then Z is not phenyl or methyl.

3. The method as recited in claim 2, wherein:
X₁ is N;
Y is selected from the group consisting of a bond, NR₁[C(R₂₀)(R₂₁)]ₙ, and NR₁[C(R₂₂)(R₂₃)]ₙ—W—[C(R₂₄)(R₂₅)]ₘ; and
W is NR₃₈.

4. The method as recited in claim 3, wherein R₁₁ and R₁₄ are each independently selected from the group consisting of hydrogen and C₁-C₃ alkyl.

5. The method as recited in claim 4, wherein:
R₁₁ is hydrogen; and
R₁₄ is methyl.

6. The method as recited in claim 2, wherein said compound has a structural formula selected from the group consisting of structural Formula III and structural formula IV: 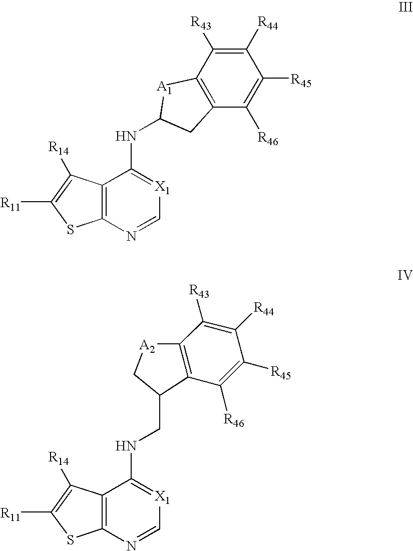
or a salt thereof, wherein:
A₁ and A₂ are each independently selected from the group consisting of a bond, —CH₂—, —CH₂CH₂—, and —CH₂CH₂CH₂—;
X₁ is selected from the group consisting of [C(R₂)] and N;
R₂, R₁₄, and R₄₃ to R₄₆ are each independently selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted; and
R₁₁ is selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted.

7. The method as recited in claim 6, wherein:
A₁ and A₂ are each independently selected from the group consisting of —CH₂— and —CH₂CH₂—;
X₁ is N;
R₁₁ and R₁₄ are independently selected from the group consisting of hydrogen and C₁-C₃ alkyl; and
R₄₃ to R₄₆ are each independently selected from the group consisting of hydrogen, alkyl, heteroalkyl, alkoxy, halogen, haloalkyl, amino, aminoalkyl, amido, carboxyl, acyl, hydroxy, cyano, nitro, and mercaptyl.

8. The method as recited in claim 7, wherein:
A₁ and A2 are —CH₂—;
R₁₁ is hydrogen;
R₁₄, is methyl;
R₄₃ and R₄₆ are hydrogen; and
R₄₄ and R₄₅ are each independently selected from the group consisting of hydrogen, lower alkyl, lower alkoxy, halogen, and lower haloalkyl.

9. The method as recited in claim 8, wherein:
said compound has structural formula III;
R₄₄ is hydrogen; and
R₄₅ is halogen.

10. The method as recited in claim 9, wherein R₄₅ is chlorine.

11. The method as recited in claim 8, wherein:
said compound has structural formula IV;
one of R₄₄ and R₄₅ is hydrogen; and
the other of R₄₄ and R₄₅ is halogen.

12. The method as recited in claim 11, wherein R₄₅ is chlorine.

13. The method as recited in claim 2, wherein:
Y is NR₁[C(R₂₀)(R₂₁)]ₙ;
n is an integer from 2 to 3;
Z is 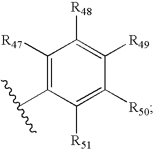
R₁, R₂₀, and R₂₁ are each independently selected from the group consisting of hydrogen and optionally substituted lower alkyl; and
R₄₇ to R₅₁ are each independently selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted; and
any two adjacent R₄₇, R₄₈, R₄₉, R₅₀, or R₅₁ may join together to form a 5-, 6-, or 7-membered cycloalkyl or heterocycloalkyl.

14. The method as recited in claim 13, wherein:
X₁ is N;
n is 2; and
R₁, R₂₀, and R₂₁ are each independently selected from the group consisting of hydrogen and methyl.

15. The method as recited in claim 14, wherein:
R₁₁ and R₁₄ are each independently selected from the group consisting of hydrogen and C₁-C₃ alkyl; and
R₄₇ to R₅₁ are each independently selected from the group consisting of hydrogen, alkyl, heteroalkyl, alkoxy, halogen, haloalkyl, amino, aminoalkyl, amido, carboxyl, acyl, hydroxy, cyano, nitro, and mercaptyl.

16. The method as recited in claim 15, wherein:
R₁, R₁₁, R₂₀, and R₂₁ are each hydrogen; and
R₁₄ is methyl.

17. The method as recited in claim 16, wherein R₄₇ to R₅₁ are each independently selected from the group consisting of hydrogen, halogen, lower alkyl, and lower alkoxy.

18. The method as recited in claim 17, wherein:
R₄₇, R₄₈, R₅₀, and R₅₁ are hydrogen; and
R₄₉ is selected from the group consisting of hydrogen, halogen, methyl, and methoxy.

19. The method as recited in claim 18, wherein R₄₉ is chlorine.

20. The method as recited in claim 2, wherein said compound has structural Formula V: 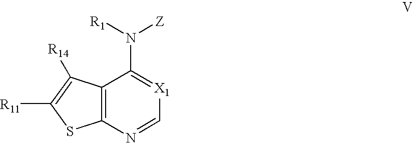
or a salt thereof, wherein:
X₁ is selected from the group consisting of [C(R₂)] and N;
Z is a 5- to 7-membered saturated cycloalkyl, which may be optionally substituted with one or more substituents selected from the group consisting of lower alkyl, lower alkanoyl, lower heteroalkyl, lower haloalkyl, lower perhaloalkyl, lower perhaloalkoxy, lower alkoxy, lower haloalkoxy, lower alkoxyalkyl, oxo, lower acyloxy, carboxyl, lower carboxyester, lower carboxamido, cyano, hydrogen, halogen, hydroxy, amino, lower alkylamino, amido, thiol, lower alkylthio, lower haloalkylthio, and lower perhaloalkylthio;
R₁, R₂, and R₁₄ are each independently selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted; and
R₁₁ is selected from the group consisting of hydrogen, alkyl, heteroalkyl, alkoxy, halogen, haloalkyl, amino, aminoalkyl, amido, carboxyl, acyl, hydroxy, cyano, nitro, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted.

21. The method as recited in claim 20, wherein:
X₁ is N;
R₁ is hydrogen; and
R₁₁ and R₁₄ are independently selected from the group consisting of hydrogen and C₁-C₃ alkyl.

22. The method as recited in claim 21, wherein Z is cyclohexyl, which may be optionally substituted with one or more substituents selected from the group consisting of lower alkyl, lower alkanoyl, lower heteroalkyl, lower alkoxy, oxo, lower acyloxy, carboxyl, lower carboxyester, and lower alkylamino.

23. The method as recited in claim 22, wherein:
Z is cyclohexyl which may be optionally substituted in the 4-position with a substituent selected from the group consisting of lower alkyl and lower alkoxy;
R₁₁ is hydrogen; and
R₁₄ is methyl.

24. The method as recited in claim 23, wherein Z is 4-alkylcyclohexyl.

25. The method as recited in claim 24, wherein Z is 4-methylcyclohexyl.

26. The method as recited in claim 2, wherein said compound has structural Formula VI: 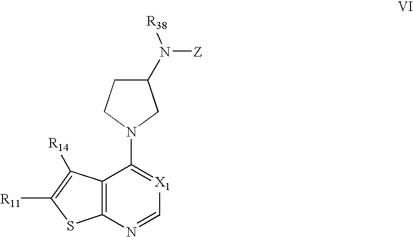
or a salt thereof, wherein:
X₁ is selected from the group consisting of [C(R₂)] and N;
Z is selected from the group consisting of hydrogen, aryl, alkyl, heterocycloalkyl, alkoxylcarbonyl, acyl, and cycloalkyl, any of which may be optionally substituted;
R₂, R₁₄, and R₃₄ are each independently selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
R₁₁ is selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted; and
R₁₁ and R₁₄ may be joined together to form a partially saturated cycloalkyl.

27. The method as recited in claim 26, wherein:
X₁ is N; and
R₁₁ and R₁₄ are each independently selected from the group consisting of hydrogen and C₁-C₃ alkyl.

28. The method as recited in claim 27, wherein:
R₁₁ is hydrogen; and
R₁₄ is methyl.

29. The method as recited in claim 28, wherein:
Z is selected from the group consisting of alkoxylcarbonyl and acyl; and
R₃₄ is lower alkyl.

30. The method as recited in claim 2, wherein said compound is selected from the group consisting of Examples 1-14, 16-87, 89-111, 113-125, 127, 129-141, 143-290, 293, 295-304, 306-313, and 316-318.

31. The method as recited in claim 2, wherein said treatment is systemic.

32. The method as recited in claim 2, wherein said administration is topical.

33. The method as recited in claim 2, wherein said disease is selected from the group consisting of an inflammatory disease, an autoimmune disease, an allergic disorder, and an ocular disorder.

34. The method as recited in claim 33, wherein disease is selected from the group consisting of pruritus, eczema, asthma, rhinitis, dry eye, ocular inflammation, allergic conjunctivitis, vernal conjunctivitis, vernal keratoconjunctivitis, and giant papillary conjunctivitis.

35. The method as recited in claim 32, wherein said topical administration is to the skin.

36. The method as recited in claim 32, wherein said topical administration is to the eye.

37. The method as recited in claim 32, wherein said topical administration is intranasal or by inhalation.

38. A method of inhibition of H₁R and/or H₄R comprising contacting H₁R and/or H₄R with a compound of structural Formula II: 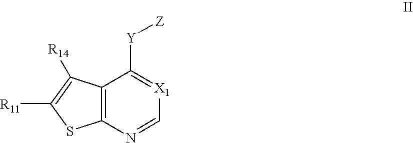
or a salt thereof, wherein:
X₁ is selected from the group consisting of [C(R₂)] and N;
Y is selected from the group consisting of a bond, NR₁[C(R₂₀)(R₂₁)]ₙ, NR₁[C(R₂₂)(R₂₃)]ₙ—W—[C(R₂₄)(R₂₅)]ₘ, S—[C(R₂₆)(R₂₇)]ₙ—W—[C(R₂₈)(R₂₉)]ₘ, O[C(R₃₀)(R₃₁)]ₙ, [C(R₃₂)(R₃₃)]ₙ—W—[C(R₃₄)(R₃₅)]ₘ, and [C(R₃₆)(R₃₇)]ₙ;
n and m are each independently an integer from 0 to 3;
W is selected from the group consisting of O, S, S(O)₂, NR₃₈, NR₃₉S(O₂), C(O), C(S), C(O)O, C(O)NR₄₀, NR₄₁C(O), and NR₄₂C(O)O;
Z is selected from the group consisting of aryl, alkyl, heterocycloalkyl, alkoxylcarbonyl, acyl, and cycloalkyl, any of which may be optionally substituted;
R₁, R₂, R₁₄, and R₂₀ to R₄₂ are each independently selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
R₁₁ is selected from the group consisting of hydrogen, alkyl, heteroalkyl, alkoxy, halogen, haloalkyl, amino, aminoalkyl, amido, carboxyl, acyl, hydroxy, cyano, nitro, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
R₁₁ and R₁₄ may be joined together to form a partially saturated cycloalkyl;
R₁ and R₂₀, or R₁ and R₂₂, or R₂₂ and R₃₈, or R₁ and R₃₈, may be joined together to form a heterocycloalkyl;
and with the provisos that;
if Y is NR₁[C(R₂₀)(R₂₁)]ₙ, R₁ is hydrogen, and n is 0, then Z is not aryl or heteroaryl; and
if Y is NR₁[C(R₂₂)(R₂₃)]ₙ—W—[C(R₂₄)(R₂₅)]ₘ, n is 2, m is 0, W is NR₃₈, R₂₂, and R₂₃ are hydrogen, and R₁ and R₃₈ are joined together to form a piperazine ring, then Z is not phenyl or methyl.

39. A method of treatment of the pain or inflammation resulting from cataract surgery, comprising delivering to a patient in need of such treatment with a therapeutically effective amount of a compound of structural Formula II: 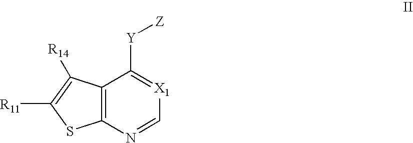
or a salt thereof, wherein:
X₁ is selected from the group consisting of [C(R₂)] and N;
Y is selected from the group consisting of a bond, NR₁[C(R₂₀)(R₂₁)]ₙ, NR₁[C(R₂₂)(R₂₃)]ₙ—W—[C(R₂₄)(R₂₅)]ₘ, S—[C(R₂₆)(R₂₇)]ₙ—W—[C(R₂₈)(R₂₉)]ₘ, O[C(R₃₀)(R₃₁)]ₙ, [C(R₃₂)(R₃₃)]ₙ—W—[C(R₃₄)(R₃₅)]ₘ, and [C(R₃₆)(R₃₇)]ₙ;
n and m are each independently an integer from 0 to 3;
W is selected from the group consisting of O, S, S(O)₂, NR₃₈, NR₃₉S(O₂), C(O), C(S), C(O)O, C(O)NR₄₀, NR₄₁C(O), and NR₄₂C(O)O;
Z is selected from the group consisting of aryl, alkyl, heterocycloalkyl, alkoxylcarbonyl, acyl, and cycloalkyl, any of which may be optionally substituted;
R₁, R₂, R₁₄, and R₂₀ to R₄₂ are each independently selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
R₁₁ is selected from the group consisting of hydrogen, alkyl, heteroalkyl, alkoxy, halogen, haloalkyl, amino, aminoalkyl, amido, carboxyl, acyl, hydroxy, cyano, nitro, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
R₁₁ and R₁₄ may be joined together to form a partially saturated cycloalkyl; and
R₁ and R₂₀, or R₁ and R₂₂, or R₂₂ and R₃₈, or R₁ and R₃₈, may be joined together to form a heterocycloalkyl.

40. A method of treatment of an H₄R-mediated disease comprising the administration of:
a. a therapeutically effective amount of a compound of structural Formula II: 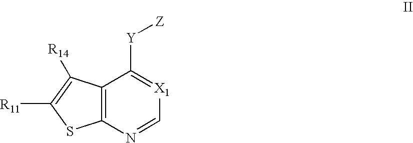
or a salt thereof, wherein:
X₁ is selected from the group consisting of [C(R₂)] and N;
Y is selected from the group consisting of a bond, NR₁[C(R₂₀)(R₂₁)]ₙ, NR₁[C(R₂₂)(R₂₃)]ₙ—W—[C(R₂₄)(R₂₅)]ₘ, S—[C(R₂₆)(R₂₇)]ₙ—W—[C(R₂₈)(R₂₉)]ₘ, 0[C(R₃₀)(R₃₁)]ₙ, [C(R₃₂)(R₃₃)]ₙ—W—[C(R₃₄)(R₃₅)]ₘ, and [C(R₃₆)(R₃₇)]ₙ;
n and m are each independently an integer from 0 to 3;
W is selected from the group consisting of O, S, S(O)₂, NR₃₈, NR₃₉S(O₂), C(O), C(S), C(O)O, C(O)NR₄₀, NR₄₁C(O), and NR₄₂C(O)O;
Z is selected from the group consisting of aryl, alkyl, heterocycloalkyl, alkoxylcarbonyl, acyl, and cycloalkyl, any of which may be optionally substituted;
R₁, R₂, R₁₄, and R₂₀ to R₄₂ are each independently selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
R₁₁ is selected from the group consisting of hydrogen, alkyl, heteroalkyl, alkoxy, halogen, haloalkyl, amino, aminoalkyl, amido, carboxyl, acyl, hydroxy, cyano, nitro, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
R₁₁ and R₁₄ may be joined together to form a partially saturated cycloalkyl;
R₁ and R₂₀, or R₁ and R₂₂, or R₂₂ and R₃₈, or R₁ and R₃₈, may be joined together to form a heterocycloalkyl;
and with the provisos that;
if Y is NR₁[C(R₂₀)(R₂₁)]ₙ, R₁ is hydrogen, and n is 0, then Z is not aryl or heteroaryl; and
if Y is NR₁[C(R₂₂)(R₂₃)]ₙ—W—[C(R₂₄)(R₂₅)]ₘ, n is 2, m is 0, W is NR₃₈, R₂₂, and R₂₃ are hydrogen, and R₁ and R₃₈ are joined together to form a piperazine ring, then Z is not phenyl or methyl; and
b. another therapeutic agent.

41. A method for achieving an effect in a patient, wherein the effect is selected from the group consisting of reduction in the number of mast cells, inhibition of eosiniphil migration optionally to the nasal mucosa, the eye, or the wound site, reduction in inflammatory markers, reduction in inflammatory cytokines, reduction in scratching, decreased watering or redness of the eyes, and reduction in ocular pain, comprising the administration, to a patient, of a therapeutically effective amount of a compound of structural formula II: 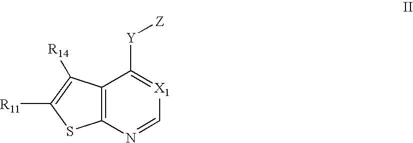
or a salt thereof, wherein:
X₁ is selected from the group consisting of [C(R₂)] and N;
Y is selected from the group consisting of a bond, NR₁[C(R₂₀)(R₂₁)]ₙ, NR₁[C(R₂₂)(R₂₃)]ₙ—W—[C(R₂₄)(R₂₅)]ₘ, S—[C(R₂₆)(R₂₇)]ₙ—W—[C(R₂₈)(R₂₉)]ₘ, O[C(R₃₀)(R₃₁)]ₙ, [C(R₃₂)(R₃₃)]ₙ—W—[C(R₃₄)(R₃₅)]ₘ, and [C(R₃₆)(R₃₇)]ₙ;
n and m are each independently an integer from 0 to 3;
W is selected from the group consisting of O, S, S(O)₂, NR₃₈, NR₃₉S(O₂), C(O), C(S), C(O)O, C(O)NR₄₀, NR₄₁C(O), and NR₄₂C(O)O;
Z is selected from the group consisting of aryl, alkyl, heterocycloalkyl, alkoxylcarbonyl, acyl, and cycloalkyl, any of which may be optionally substituted;
R₁, R₂, R₁₄, and R₂₀ to R₄₂ are each independently selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
R₁₁ is selected from the group consisting of hydrogen, alkyl, heteroalkyl, alkoxy, halogen, haloalkyl, amino, aminoalkyl, amido, carboxyl, acyl, hydroxy, cyano, nitro, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
R₁₁ and R₁₄ may be joined together to form a partially saturated cycloalkyl;
R₁ and R₂₀, or R₁ and R₂₂, or R₂₂ and R₃₈, or R₁ and R₃₈, may be joined together to form a heterocycloalkyl;
and with the provisos that;
if Y is NR₁[C(R₂₀)(R₂₁)]ₙ, R₁ is hydrogen, and n is 0, then Z is not aryl or heteroaryl; and
if Y is NR₁[C(R₂₂)(R₂₃)]ₙ—W—[C(R₂₄)(R₂₅)]ₘ, n is 2, m is 0, W is NR₃₈, R₂₂, and R₂₃ are hydrogen, and R₁ and R₃₈ are joined together to form a piperazine ring, then Z is not phenyl or methyl.

42. A compound, for use in the manufacture of a medicament for the prevention or treatment of a disease or condition ameliorated by the inhibition of H₁R and/or H₄R, of structural formula II: 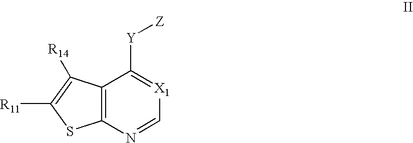
or a salt thereof, wherein:
Xᵢ is selected from the group consisting of [C(R₂)] and N;
Y is selected from the group consisting of a bond, NR₁[C(R₂₀)(R₂₁)]ₙ, NR₁[C(R₂₂)(R₂₃)]ₙ—W—[C(R₂₄)(R₂₅)]ₘ, S—[C(R₂₆)(R₂₇)]ₙ—W—[C(R₂₈)(R₂₉)]ₘ, O[C(R₃₀)(R₃₁)]ₙ, [C(R₃₂)(R₃₃)]ₙ—W—[C(R₃₄)(R₃₅)]ₘ, and [C(R₃₆)(R₃₇)]ₙ;
n and m are each independently an integer from 0 to 3;
W is selected from the group consisting of O, S, S(O)₂, NR₃₈, NR₃₉S(O₂), C(O), C(S), C(O)O, C(O)NR₄₀, NR₄₁C(O), and NR₄₂C(O)O;
Z is selected from the group consisting of aryl, alkyl, heterocycloalkyl, alkoxylcarbonyl, acyl, and cycloalkyl, any of which may be optionally substituted;
R₁, R₂, R₁₄, and R₂₀ to R₄₂ are each independently selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
R₁₁ is selected from the group consisting of hydrogen, alkyl, heteroalkyl, alkoxy, halogen, haloalkyl, amino, aminoalkyl, amido, carboxyl, acyl, hydroxy, cyano, nitro, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
R₁₁ and R₁₄ may be joined together to form a partially saturated cycloalkyl;
R₁ and R₂₀, or R₁ and R₂₂, or R₂₂ and R₃₈, or R₁ and R₃₈, may be joined together to form a heterocycloalkyl;
and with the provisos that;
if Y is _{NR}₁[C(R₂₀)(R₂₁)]ₙ, R₁ is hydrogen, and n is 0, then Z is not aryl or heteroaryl; and
if Y is NR₁[C(R₂₂)(R₂₃)]ₙ—W—[C(R₂₄)(R₂₅)]ₘ, n is 2, m is 0, W is NR₃₈, R₂₂, and R₂₃ are hydrogen, and R₁ and R₃₈ are joined together to form a piperazine ring, then Z is not phenyl or methyl.

43. A compound having a structural formula selected from the group consisting of structural Formula III and structural formula IV: 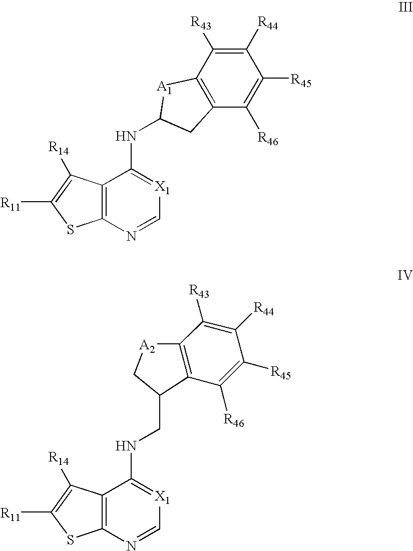
or a salt thereof, wherein:
A₁ and A₂ are each independently selected from the group consisting of a bond, —CH₂—, —CH₂CH₂—, and —CH₂CH₂CH₂—;
X₁ is selected from the group consisting of [C(R₂)] and N;
R₂ is selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
R₁₁ is selected from the group consisting of hydrogen, alkyl, heteroalkyl, alkoxy, haloalkyl, amino, aminoalkyl, amido, carboxyl, acyl, hydroxy, cyano, nitro, arylalkyl, cycloalkyl, cycloalkylalkyl, heterocycloalkylalkyl, heteroaryl, heteroarylalkyl, mercaptyl, alkylsulfonyl, sulfonamide, and alkylsulfonamido, any of which may be optionally substituted;
R₁₄ is is selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
R₄₃ and R₄₆ are each independently selected from the group consisting of hydrogen, alkyl, heteroalkyl, C₂-C₆ alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
R₄₄ and R₄₅ are each independently selected from the group consisting of hydrogen, alkyl, heteroalkyl, C₂-C₆ alkoxy, halogen, haloalkyl, amino, aminoalkyl, acyl, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted; and
with the proviso that;
if the compound has structural formula III, A₁ is —CH₂—, R₁₁ is hydrogen or methyl, and R₁₄ is hydrogen, methyl, or isopropyl, then at least one of R₄₃ to R₄₆ is not hydrogen.

44. The compound as recited in claim 43, wherein:
A₁ and A₂ are each independently selected from the group consisting of —CH₂— and —CH₂CH₂—;
X₁ is N;
R₁₁ and R₁₄ are each independently selected from the group consisting of hydrogen and C₁-C₃ alkyl; and
R₄₃ to R₄₆ are each independently selected from the group consisting of hydrogen, alkyl, heteroalkyl, C₂-C₆ alkoxy, halogen, haloalkyl, amino, aminoalkyl, amido, carboxyl, acyl, hydroxy, cyano, nitro, and mercaptyl.

45. The compound as recited in claim 44, wherein:
A₁ and A2 are —CH₂—;
R₁₁ is hydrogen;
R₁₄, is methyl;
R₄₃ and R₄₆ are hydrogen; and
R₄₄ and R₄₅ are each independently selected from the group consisting of hydrogen, lower alkyl, lower alkoxy, halogen, and lower haloalkyl.

46. The compound as recited in claim 45, wherein:
said compound has structural formula III;
R₄₄ is hydrogen; and
R₄₅ is halogen.

47. The compound as recited in claim 46, wherein R₄₅ is chlorine.

48. The compound as recited in claim 45, wherein:
said compound has structural formula IV;
one of R₄₄ and R₄₅ is hydrogen; and
the other of R₄₄ and R₄₅ is halogen.

49. The compound as recited in claim 48, wherein R₄₅ is chlorine.

50. A compound of structural Formula II: 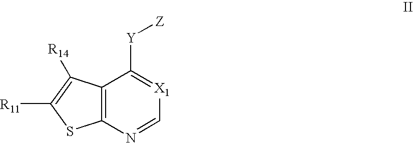
or a salt thereof, wherein:
X₁ is selected from the group consisting of [C(R₂)] and N;
Y is NR₁[C(R₂₀)(R₂₁)]ₙ;
n is an integer from 2 to 3;
Z is 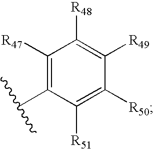
R₁, R₂₀, and R₂₁ are each independently selected from the group consisting of hydrogen and lower alkyl;
R₁₁ and R₁₄ are independently selected from the group consisting of hydrogen and C₁-C₃ alkyl;
R₂, R₄₇ to R₅₁ are each independently selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted;
any two adjacent R₄₇, R₄₈, R₄₉, R₅₀, or R₅₁ may be joined together to form a 5-, 6-, or 7-membered cycloalkyl or heterocycloalkyl;
with the provisos that;
if X₁ is [C(R₂)], R₁, R₂, R₂₀, and R₂₁ are hydrogen, R₁₁ is ethyl and R₁₄ is hydrogen, then at least one of R₄₇ to R₅₁ is not hydrogen;
if X₁ is N, then at least one of R₂₀ and R₂₁ is lower alkyl; and
if X₁ is N, R₁₁, R₁₄, and R₄₇ to R₅₁ are hydrogen, then Y is not —CH₂C(CH₃)₂—.

51. The compound as recited in claim 50, wherein:
X₁ is N;
n is 2; and
R₁, R₂₀, and R₂₁ are each independently selected from the group consisting of hydrogen and methyl.

52. The compound as recited in claim 51, wherein R₄₇ to R₅₁ are each independently selected from the group consisting of hydrogen, alkyl, heteroalkyl, alkoxy, halogen, haloalkyl, amino, aminoalkyl, amido, carboxyl, acyl, hydroxy, cyano, nitro, and mercaptyl.

53. The compound as recited in claim 52, wherein:
R₁ and R₁₁ are each hydrogen; and
R₁₄ is methyl.

54. The compound as recited in claim 53, wherein R₄₇ to R₅₁ are each independently selected from the group consisting of hydrogen, halogen, lower alkyl, and lower alkoxy.

55. The compound as recited in claim 54, wherein:
R₄₇, R₄₈, R₅₀, and R₅₁ are hydrogen; and
R₄₉ is selected from the group consisting of hydrogen, halogen, methyl, and methoxy.

56. The compound as recited in claim 55, wherein R₄₉ is chlorine.

57. A compound of structural Formula V: 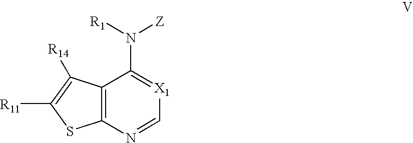
or a salt thereof, wherein:
X₁ is selected from the group consisting of [C(R₂)] and N;
Z is a 5- to 7-membered saturated cycloalkyl, which is substituted with at least one substituent selected from the group consisting of lower alkyl, lower alkanoyl, lower heteroalkyl, lower haloalkyl, lower perhaloalkyl, lower perhaloalkoxy, lower alkoxy, lower haloalkoxy, lower alkoxyalkyl, oxo, lower acyloxy, lower carboxyester, lower carboxamido, cyano, hydrogen, halogen, hydroxy, thiol, lower alkylthio, lower haloalkylthio, and lower perhaloalkylthio;
R₁ and R₂ are each independently selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, and alkylsulfonamido, any of which may be optionally substituted;
R₁₁ and R₁₄ are independently selected from the group consisting of hydrogen and C₁-C₃ alkyl;
with the provisos that;
if R₁₁ is methyl and R₁₄ is hydrogen, then Z is not 2,3-dimethylcyclohexyl;
if R₁₁ and R₁₄ are both hydrogen, if R₁₁ and R₁₄ are both methyl, or if R₁₁ is ethyl and R₁₄ is hydrogen, then Z is not 4-hydroxycyclohexyl;
if R₁₁ and R₁₄ are both hydrogen or if R₁₁ and R₁₄ are both methyl, then Z is not 2-methylcyclohexyl;
if R₁₁ and R₁₄ are both hydrogen or if R₁₁ and R₁₄ are both methyl, then Z is not 3-methylcyclohexyl; and
if R₁₁ and R₁₄ are both hydrogen or if R₁₁ and R₁₄ are both methyl, then Z is not 4-methylcyclohexyl.

58. The compound as recited in claim 57, wherein:
X₁ is N; and
R₁ is hydrogen.

59. The compound as recited in claim 58, wherein Z is cyclohexyl, which may be optionally substituted with at least one substituent selected from the group consisting of lower alkyl, lower alkanoyl, lower heteroalkyl, lower alkoxy, oxo, lower acyloxy, carboxyl, lower carboxyester, and lower alkylamino.

60. The compound as recited in claim 59, wherein:
Z is cyclohexyl which is substituted in the 4-position with a substituent selected from the group consisting of lower alkyl and lower alkoxy;
R₁₁ is hydrogen; and
R₁₄ is methyl.

61. The compound as recited in claim 60, wherein Z is 4-alkylcyclohexyl.

62. The compound as recited in claim 61, wherein Z is 4-methylcyclohexyl.

63. A compound of structural Formula VI: 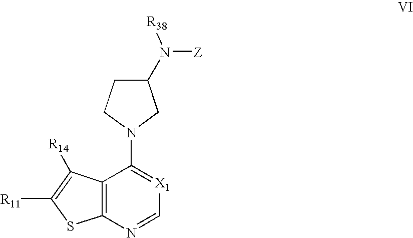
or a salt thereof, wherein:
X₁ is selected from the group consisting of [C(R₂)] and N;
Z is selected from the group consisting of hydrogen, aryl, alkyl, heterocycloalkyl, alkoxylcarbonyl, acyl, and cycloalkyl, any of which may be optionally substituted;
R₂, R₁₄, and R₃₄ are each independently selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted; R₁₁ is selected from the group consisting of hydrogen, alkyl, heteroalkyl, alkoxy, halogen, haloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, heteroarylalkyl, mercaptyl, alkylsulfonyl, sulfonamide, and alkylsulfonamido, any of which may be optionally substituted; and
R₁₁ and R₁₄ may be joined together to form a partially saturated cycloalkyl.

64. The compound as recited in claim 63, wherein:
X₁ is N; and
R₁₁ and R₁₄ are each independently selected from the group consisting of hydrogen and C₁-C₃ alkyl.

65. The compound as recited in claim 64, wherein:
R₁₁ is hydrogen; and
R₁₄ is methyl.

66. The compound as recited in claim 65, wherein:
Z is selected from the group consisting of alkoxylcarbonyl and acyl; and
R₃₄ is lower alkyl.

67. A pharmaceutical composition comprising a compound as recited in claim 43 together with a pharmaceutically acceptable carrier.

68. A pharmaceutical composition comprising:
a. a compound as selected in claim 43;
b. a H₁R antagonist; and
c. one or more pharmaceutically acceptable carriers or adjuvants.

69. The pharmaceutical composition as recited in claim 68, wherein said H₁R antagonist is selected from the group consisting of acrivastine, alcaftadine, antazoline, azelastine, bromazine, brompheniramine, cetirizine, chlorpheniramine, clemastine, desloratidine, diphenhydramine, diphenylpyraline, ebastine, emedastine, epinastine, fexofenadine, hydroxyzine, ketotifen, levocabastine, levocetirizine, loratidine, methdilazine, mizolastine, promethazine, olopatadine, and triprolidine.

70. A pharmaceutical composition comprising:
a. a compound as selected in claim 43;
b. a H₃R antagonist; and
c. one or more pharmaceutically acceptable carriers or adjuvants.

71. A pharmaceutical composition comprising:
a. a compound as selected in claim 43;
b. a H₁R antagonist and a H₃R antagonist; and
c. one or more pharmaceutically acceptable carriers or adjuvants.

72. A compound as recited in claim 43 for use as a medicament.

73. A pharmaceutical composition comprising a compound as recited in claim 50 together with a pharmaceutically acceptable carrier.

74. A pharmaceutical composition comprising:
a. a compound as selected in claim 50;
b. a H₁R antagonist; and
c. one or more pharmaceutically acceptable carriers or adjuvants.

75. The pharmaceutical composition as recited in claim 74, wherein said H₁R antagonist is selected from the group consisting of acrivastine, alcaftadine, antazoline, azelastine, bromazine, brompheniramine, cetirizine, chlorpheniramine, clemastine, desloratidine, diphenhydramine, diphenylpyraline, ebastine, emedastine, epinastine, fexofenadine, hydroxyzine, ketotifen, levocabastine, levocetirizine, loratidine, methdilazine, mizolastine, promethazine, olopatadine, and triprolidine.

76. A pharmaceutical composition comprising:
a. a compound as selected in claim 50;
b. a H₃R antagonist; and
c. one or more pharmaceutically acceptable carriers or adjuvants.

77. A pharmaceutical composition comprising:
a. a compound as selected in claim 50;
b. a H₁R antagonist and a H₃R antagonist; and
c. one or more pharmaceutically acceptable carriers or adjuvants.

78. A compound as recited in claim 50 for use as a medicament.

79. A pharmaceutical composition comprising a compound as recited in claim 57 together with a pharmaceutically acceptable carrier.

80. A pharmaceutical composition comprising:
a. a compound as selected in claim 57;
b. a H₁R antagonist; and
c. one or more pharmaceutically acceptable carriers or adjuvants.

81. The pharmaceutical composition as recited in claim 80, wherein said H₁R antagonist is selected from the group consisting of acrivastine, alcaftadine, antazoline, azelastine, bromazine, brompheniramine, cetirizine, chlorpheniramine, clemastine, desloratidine, diphenhydramine, diphenylpyraline, ebastine, emedastine, epinastine, fexofenadine, hydroxyzine, ketotifen, levocabastine, levocetirizine, loratidine, methdilazine, mizolastine, promethazine, olopatadine, and triprolidine.

82. A pharmaceutical composition comprising:
a. a compound as selected in claim 57;
b. a H₃R antagonist; and
c. one or more pharmaceutically acceptable carriers or adjuvants.

83. A pharmaceutical composition comprising:
a. a compound as selected in claim 57;
b. a H₁R antagonist and a H₃R antagonist; and
c. one or more pharmaceutically acceptable carriers or adjuvants.

84. A compound as recited in claim 57 for use as a medicament.

85. A pharmaceutical composition comprising a compound as recited in claim 63 together with a pharmaceutically acceptable carrier.

86. A pharmaceutical composition comprising:
a. a compound as selected in claim 63;
b. a H₁R antagonist; and
c. one or more pharmaceutically acceptable carriers or adjuvants.

87. The pharmaceutical composition as recited in claim 68, wherein said H₁R antagonist is selected from the group consisting of acrivastine, alcaftadine, antazoline, azelastine, bromazine, brompheniramine, cetirizine, chlorpheniramine, clemastine, desloratidine, diphenhydramine, diphenylpyraline, ebastine, emedastine, epinastine, fexofenadine, hydroxyzine, ketotifen, levocabastine, levocetirizine, loratidine, methdilazine, mizolastine, promethazine, olopatadine, and triprolidine.

88. A pharmaceutical composition comprising:
a. a compound as selected in claim 63;
b. a H₃R antagonist; and
c. one or more pharmaceutically acceptable carriers or adjuvants.

89. A pharmaceutical composition comprising:
a. a compound as selected in claim 63;
b. a H₁R antagonist and a H₃R antagonist; and
c. one or more pharmaceutically acceptable carriers or adjuvants.

90. A compound as recited in claim 63 for use as a medicament.
